SEQUENCE LISTING (8 sequences)

SEQ ID NO: 1 - DNA, 161613 bp, Homo sapiens
  source 1..161613: mol_type = genomic DNA
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattctgtgtttctactctgggagctgaggcaggagggttgcttgagcccaggaggttgaggctgcactgagctaagatggtgccactgcactccagcctgggcatcagagcaagatcctgtctctattaaaaaaaaacaacaaacaacaacaaaacacagttaagttcaacaaagatgacaaagctgcataatagcatgaccccagttttgtttttaagaagaaatgtacatgtatttacacaaaaaagaaagcacaaggcagaaagatatgttccaaatatttagaaaacattgacaattggtcattcgttgacgctgatggaattacaggtgatttttacttttctccttgtgctgctttgtattttctaaaaagaatttctattgctctttttgaaaagatgatacaacaaacattgagaaaccatcattcaaaagagattgtcttctcaaatgaatgacttacactaagactcagtgttctattgtcttcaacccgagttacatcaatgactaagtaaaagccaaagaaagccacttgatacttttatgaagtgtaaggctaaaggaaagctgaagcagatttcttatttatttatttatttatttatttatttatttttgagatggagtctcgctctgttgccccggctggagtgcagtggcacgatctcggctcactgcaagctccgcctcccgggttcaagcaattctcctgcctcagcctcccgagtagctgggactacaggcacctgccaccacgcccggctaattatttgtacttttagtagagatggggtttcaccatcttggccaggctggtcttgaactcctgacctcatgatccacctgcctcggcctccgaaagtgctgggattacaggtgtgaggcactgtgcccagccgctgaagcagatttctatgttcttagcatagctttcttccttgatgtaagtaaaatgctttgttacaatctctatgtgtattgacttttccttcccatgatgactttatctagtttaacatttcccaagatgacttcatctagtctggctttatttttttcagatgttagctaggtgagctcttccttctggaaatttccactgcacctaatacagtgacattgctaggatggctggactaagttgtggctccctctccttttagtacagcttttcctgactccgtgtccttgcaaccaaggtgaaggagatgagaaaaagctttctcagttggatttagctgatggatgtgggtcctttacaataaaataccaccctcgcaaggagggagaaatgatgtgtttctcaaagtgctgcagaagtctcattgttgatattttcctcgttgttgctttgaaaatccaatgtttctatttaacaccagagtcacccaatctaattctgccattcatgacttttgaggaatggggtggaatttggggactgaatttatgaagaggcagcatggtagagaggaaagtcctgacatcaacctctgggcggggcgttgattggcatctgtggaatgcccaccggggaaagattcttttacacgcattatcttaattctcagagcatctctgaggctttgttgcaacttcagggttttttaaaacttttattttaggtttaggggtggatgtgcaggtttgttatataagtaaattgtatgtcactggagtttggtgtacagattattttgtcacccagataataagcatagtattcgataggtagtttttcgatcctctccctcctcccacccactatcctcaagtaggtacctgtgtctgttgttcccttctttgtgtccatgtgtactcaatgtttagctcccacttatcagtgagactgtggtatttgatcaattccaattttgaccaatggtgaccagagacacagagcagttgggtgtggtggagacagggttcaagtgtaagtcaacctagctggtaaagtgacacccatcccactcatcccattgctgccaatgacttcaacgctggagcctcagttccctcccgtatgagatgatgataacaattgtgaagtgtctgaggatcagagaaataacccagtgaaggtacttcagagtgggtaacttttaattactgttgtattgctgtactgaccaatgtagtcagtagccacatgtgcctacttaaattaaattaaacaaaaattccatttctcagctacactagctccatttccagtgctcaatggcctcctacctgtagtagctgccatattggatagtacagacacagaacattcccatcgccacagaatgtcccttggacagtgctgctctgtatcattaactttgatttaattcaagtgccagacctcagggctgaacacaatcattccctatgtgtgaatgtgcaggcacctgtgcagttttgttgtataggtaaattgtgtgtcactggagtttggtgtacagattattttgtcacccagataataagcatagtattcgataggtagtttttcgatcctctctctcctcccaccctctaaccttaagtaggtaagaggaagctccgaagctctcctgggatgttcacccgctgtggtgagagtcaggtgaggcccttcacggagggcaccctttgtttaccagagagaagggagtcacgtttgtttcctgaatgaagatagcctatctaagaaccaggtatctcagcctcgaaaaggtgggaagtgcatgtcaaggaaacctctagacaggactcaattcttcccatcatacacgtaaccagagaatcgcagaaggggtcggggaggattccaactttgagtcagcaaagaagacattcttttgaggtagagtcagagtagggaaaacaactaatctgttttccaaacaggttttcataaataactgacaggagatgggctgtttttatggaagtattaacccaaatgatgggtgttaatcattaaacctttttggtggtggctcgcttgtttctctgatattctaatccgggtcacagaatggctaaggcagatttaagtgcagcgtggattttttttttctcactttgccttgtgttttccaccctgaaagaatgttgtggctgctcttttttctggtgactgccattcatgctgaactctgtcaaccaggtatgtagccttgaaatttaaaaatagatgaacagttttctacatgtttgacaagtcatcgactgaaagaactctttatttttctaaatctgcagacatgttgtggcttgaaagttggggcagtgagtgtttaatgagtttgaaagaaaatattaaaccatctaagatgatttaggttaaacttccaggagtatttggggctgtttaattgtagttctttgttccattagtttgagttgcattacatattaggatttttcagactctgttttggcagatcctaatcatcatatactgctctttcttttttcttttctggcaggtgcagaaaatgcttttaaagtgagacttagtatcagaacagctctgggagataaagcagtaagtggagcacctgagtgttctttttaaattttgtttgctaataacactgtgctttctctaataatgggcctgtctgcatagttaccttaatcttgaataaattatttctaaagacctcaaatctgatttcttaaagatagattatagaataatatgaatttttaataaagtaggtttgcctaaagtatttaatcagtagtgtatgctaggcaacatgtgaagttttgtgccacacaaaacaacttaaaaaaccagcatctcaactcgggatagtgaagccaagtggcctgagtcagtcgttcaggtctggactgggtctactcctcaccgttgtggtcatttgaattttgttagcctcgattttctaccattgaaaataagggatagttctacccacataaagtctttcatagcacaaaaaatttccaatttgaacctgaagtttcttcaattattctcaattatcaaagtaacatatgaaatatatgcttatgaaatagaagtttaaaaaatagtcatagggcccatgtggctataggaatggtaaagatcattgaattaaaaagacagtattgtctaattctagtttcagtttcctagtgtggtctgcaaaattttttccccaatagaaggtgacgttaagatgccttaggcatagaaacagaaaaaagagtgctaaagaaacttagaggtattctgtttgcaggccagttcatcaatttggtattttattattgactcaatgagtaatgattgttataacagccagcatttattgagaacttatcttactttctagctggggctcttctaagcaccttactcctatcaaatcatttagccctcacaacaatgctttgaggaagatagtagtacaggtattagtattcccactgaatggatgagaaaactgaggttcaaagaaattaagtaacatgctcaatatttcaagtcagcaagtggcagaagtaagatttgagcccaggcagtctgagtgcagaatacactgtctctactagtgtgcagggttggccatcccagtatcctctaatagcaggtacgatgctgaatgctgggatgtaatggtgattaaggcttgtccttacccttgtggagcttatagtagtagtagaattggtcatctattaaatattcagtaaaccaaatgcaaatgtgcaatcttgacaagtattacgggaagaagtacaagggcacttcggctttgtcaaggcccacaatacctggcacttactgtgatgtctgaagcatgagttgaagtgttacaggaaatgaaagaaggaaaggcagagggaacagcatgtgcaaaggcttatcacaggaaggatcttggtgagagtaaagattagaaagagggcagaaatatctggaattggggcagtgaggaggaacatggcaggtgatgagcttaaagagagaggagcaggcaggccaaggaatatgtgggctgtgttagggagtttcattttgagatgtagggagggttcaagagcatcaaatgccgttgagaggccaagccagaaggcgactgaaaaatgttcattgtgttggacaatatggaggtcaatggtgattttagaaagagctcttttcgtggggtggaggagctggaagacagatagtggggagttaaaaaataaatgagtggtgaacatgttgaaacagatgtgtggacaactcctgggagaattttggctgtgaagggcaggaaagagatagcagtaggtagaagaaaggaagactgagtaaaatgatttgtttttgctgttgcaaaacaacagctctttgctatggctttaatgagaaagtcttgagcttggttaaaagacaatgaacagactccggtcagagctaaatacacagaatggaggggaactgatattgtttagatctgagaagggcagaggagaagcattcctaatataagtgacggaaatggcctctgataggcatagttctgtctggcacttcaggagaggaggcggctaagacgtgtctatggaggaagtgttgtatcttgatgtcaggaggttgggagagttgaaattgatgagatgtatctataaattatgaagcaaaaccatttagtgagagtgaggtttgagggaaaccattgggaaaaatggttaaaaaaaaaaagcattgaccatcagttctctctctagagagaggtttgaaatagttatgtgacccatgagaaagagaactgatggtgaaaccttagggattcccaggcaatttgggggtaggtcatcatgaatttagagttaaaccaactaaccctgttctgtaattgtctctagttataccagttacttacctaaaatgtggggttagccatggtttgagtgttcttagatgtgtgcaaaaagcaaggtgagctggggcatttaaggtcctggcacatgttattgaaatgaggaaccaaggaatctaaactgatagataaatatatgcaattaatttgtaccatttggtctgtgaagccacacgtggcatataaagatgtgttcaatttcacattgagcacctattatgtgtcaagtatacgcacaacaggtagttaagctgctatggacattcttatatgtgtatttttcatatgccttcttttttgggggtatttaccctggagtagattaatggatcataaagtatacaaatgtttagctttagtaactcttaccttatcctctcttaagttgtacattttctcattaatatatgtgctcaattccttttataagcggaggctctctctttgaagtgtttgaatgccctgggagaaatacactacgtccaatgcacaagctttcaatccttttcacacttgcccaagataagttggttccactggcaggatacccaacccaatctagaactgcatcagttctgttctgatctaagtagcttggacctttgttctgtgaccaaaattggactcctacccagccagcttatgctcttgtttttgagatgaacaaatggaaggacaggagatcaggtcaagttgtccccaagatctgaaaagcaactgaaattcatgtcccagggagagctggctaacatctttattttcattttctgcactgagcttattattctcatttgttcttgagcaggcttattccttgtgtctgcattctgttaataagccttccctgtcttaatagtgtttctgcaccatttttaatcagacccatttcttcttcatgttttattcatttattattaggttagatcatagctactcttattctgaataaaattaattttggaaaaaaatgcattatcaaaaacaaagagctatgccaccaatcagttacttttaaactttcatagtataatatcaaatgaaaagttcacatcaaaacattgaaggagcacttcaaataagatctaaagttgacattaaaatgcagtatttaacatttgcatatgagaaaagtgatttttccatttccattcttttgttgttgttgttgttgttgttgttgttgttgttgttggagacagggtctcactctgtcatccaggctggagtgcagtggcatgatcttggctcacggcaacctccacttcccaggttcaagcaatcttcctgccttagcctcctgggtagctgggactacaggtgcatgccaccacgcctggctgattttttttttttttttggtagagatggggtttcactatgttgcccaggctagtcttgagctcctgggctcaagtgatccacccatcttggcctcccaaagtgttacaggcttgagccaccactcctgtcccccttttctgttctttatggtaacatcttacagcagagttacataaggtaggactgaaggagaaaatggtgaagtggaagaagcaactgaagaaagtttaaggttctatgttacagaggccaaggcaggaggatcacttaaggccacacatttgagaccagcctggacagcacagcaagaccccatccctcctcccttccaaaaaaagaatatgctatgaaaatatttaggagaaaggaaaaagtacagcacagtcataaaccttacccagagaaggtaactgagtaatacattttttgttgttgttatttaggagtcatttatttttacttactatcctccctgccaccaaagaggagaaaggcagtaacggtaaaatatgtttccccatcccagagtgaaaccccaatcctgacaatgtccttgtgatataccaaccccccaccctcaccaacttcttccgctcagcttttgctactttctcccttcctgcttctctttagttgctaccctccttggtggtccttaaacagctcagtttctttttgcctcaggctttttgcatttactgcattaaggagcagagtgagtcaaatatactatattaatttgctagggttgccataacagagtaacacagactgggtgccttacacaacggaaatttattttctctcagttctggaagtcaagatgtcatagggttggttccttctgagggctgtgaaggaaggatctgttccaggcctctctctttggcctgtatgtagatggctgtcttctccctgtgtcttcatatcatcttccctctgtgtccagatttcctcttctaaggacaccaatcatagtggattagggcccactccaacaacttcattttgacttaatcatcactgcaaagaccctgtctccaaatagagttaaattctgaggtattgcaagttaggacttcaacatgaaatttttctggtgaggacaaaaattcagcccataatatgcacgcacacacactaaagtttctggctaagaaaattttttaaaaattgtactgatgttaataaaggcaactgggagggagtttatgagggaaaagagaaagggaggaaggaaatgaaaatggactatatatggccaggatgaaatgtccaaatgccctatttgttggctgggtactgatgctgcaggttcttaggaagatagtgcaacaggagacacaccaaacttagaatgtccaagaaaccctaccccaaacatggggatataatttatcgtggtctccagtatctaatatatgagaaacgtgtgtgtgtgtgcgtgtgtgtgtgtgtgtgtgtggagagagagagagagggagagccactgtgggtacaagtagaaccaatggaccatattttcccccatacactttgttttccttctttttcagtatgcctgggataccaatgaagaatacctcttcaaagcgatggtagctttctccatgagaaaagttcccaacagagaagcaacagagtaagcaaataatgagaaatcttaactgattatttacagtattatttgagatagtgcattatttcaaaccttgattttgaaaataatttattaagctaatttttcaaagggattcacacttttattaaacttaaaaataacttttataatctaactcatcttacagtttatatcatgttccaacaatgctctttgtttaatagataatatgaagataaatatccctcaaaattgcagctatatatcctaagctttctacatttctattgtgtgtgtgtgtgtatatatatatatatatatatataattttcttgcagatagataccaaagaattttactcttattttgaaaataacctaagtgtgtataagttctttccactcttggtatttatcatagtaccttgtttattttcttaaaagcacttaatatgacttttaatagttttatattttttgtctgttcttctggaggaatcttctctatgatgtttctttctttctctatttgtgatgagggtaacattcctggtctttctctattcccttcttcactcctcccttgagaggtaaaatgacttatgtgattggtgagctggaaggagaacctgggcatgggttcctttctccttgtgaggtagccatccccaaatgatagcagacgtctctcatgcaactctctagctaagtacagaaccaggccctgcatgcacaggtgcatacaccctaaggttgtacttgctgtttgcagctgaggagcatttagtaggtgccattttatatgaagggttaaatatcttgtgtattactccagtacaggacaatacctgcctcacttatgctctacttgagacaatggaggtgaatatatttcatttcaggattttttttaaattagggagaaattctgcctgctgattaaagtcatatcctccaacctaaccttttattttggtctcccatctgtacaatctttttgtcttaattctataataattcaaggcccagtatagatgattactattaattggtctccctcacatccacaatatctgatgcatgggatatgaatataaagaatgaaacagaagagtggaatttgagggagcaaaatgaacacatatacatttgtgtatacacacatgtatattttaaatatatgttttaaaataatatattaacgtgattcaaaaaatgaaaagaaggtatataataaaacaaaactttgccctaagttctactcctgttttatagatcttaaaattatatatattgtatttcatgccattaaactgcagcattctttcacaactatttataataacttttttcaataaaaataaggtgattacattacaaaaagttgaacaggaaaaaaaaaataagaagaaaagccactggttatctcacaatggtaacctcttaaatattctctttgtattttcttctagcccttcttgtaagcaagttttatacattgctgtgttcatggcactattagttcctttaaatgatcacatttcacctagtacatgactaagttaaattaagtgtttccattcttaggaaattttaatttaagtatataacatttcagattgatttttgggagtttacctttaaaatatcttccttcagaatctggagcattgtaggagtctatgaatatctgatccggtttatttgaaaggttgtttgacttacagttgggcactggggctgggtgcagtggctcacgcctccaatcccaaaactttgggaggccgaggcaggcagatcacttgagcccaggagttcgagaccagcctgggcaacatggacaacatggcaaaacctcgtttctacaaaaaatgcaaaaattagctgggcatggtggcacacaccaatagtcccagctactcaggaggctgagatcacttgagcctgggagtttgaggctgtagtgaactatgatggtgccactgcactccagcctgggtgacagagtgagactctgtctcaaaaaaaaaaaaaaattgggtacacaggatatatgtcgggttggtgaaatagtaattgtggtttttgccattgcaagtaatggcaccttcaagaaaagtagaagcttttgttgccagtgtcagttgtaaaaatcaaaactgcaaaactggactgagggtcagtacacatttttctgtaaagagtcaggcagtatgagtcctggtaatactgctatcaaggggatgaattggcgagcgagatgtgtttaagatgcaattcagctgctggcctggattccacatgcactgccatattggaagctgcacccctacatgaagagctatgggaaagggcaccacataatgcaagattggattgaaaatagctttgcggagaggctgcttctctccagtgtggcaggtggatggaagataccagtagccgccttccagaaatcttttggtgccaatgtgggcagactgaaggagtgtggcaaatattcctttccaggatagcttcaggatccaagttttgggactggctgtttattgtctcccccccgcccaccattgtttatctactaatattgcagaaaactgtaaagcacaaaaataaaccaaatcacgtgaattaaaaataaagatattcaagtaattaacagaataatgaatagtctcactttcatcccaccttttacccagggtgatatctacaacatctcttatatcaagtttggtcaccaccaatgatacagagctcattgtctcctgagcatgcctcttcctatttggaaacgtacaattggcatatacttacagtagaagcttgcatggctatgaaaaaggaagtgatgtcttgttggagagctgatctagtccagttaccatcaccgcaggcaggcagtggaagagaaaccagggattccctaaggggatcagagaggctgagggtgtgggcttgcagagagggctgtggcatttccactcccctaggacttgaacagggctgggaatgggttcctgtgcagattccccagtgcggccagctagggtgttgacaactgagttcatgctgatgagttggtccccattccttacgagtgatgatgtgttggagtgatattcttgtttgcatccctacctacctgaagtcaggtagagaagtgctcatagaggatacagttgtctttaagaaaggagactggcataatattttctggagggatgctccccaacttgcagacattctcaggactccgtcaagttgccactagagtaacagtagctggagctctagagaatatgatggtgtccctagccactgagagtgtatctggaacagaaactggtggctacttcttagccagagaagtctcatggtggaaggctggcttgagtacctgtgactggggagagagagagcagttttttgagcagtctgcacttgaatatattcctggggccaagcagaggctttggaagggaacaggatagagttactgcctattgggcccatgtccctaagtgagacagagcaaggcaaggcagcagagtgaggctggaggtgtacaccccggaggcagagccgaggcgaggtggaggaaatcagtcagcgtgtcacccatgtcaggggaagatgcagtgcagaggtccctggaggggcccttgaaggccccacaggagcattctgagtggagagcatctggacgtccactaccacagaagacctctcaaagcgggggaaccacaaggacccaggtccaggaagaataatttggcctttccttatctatctcacgcccccatcatcttgctttcctgtccctaagcgaggagaatgaagcatggtggaggggagcacaggcagggagggagtagggagtggagcccggaggaaatggctggagcccaccactcaggtccctgctctggccagaactgctggaaggagagcagctgtaggtgagaggcaggaaggcagttttggttagactggcctggatgccttagcagccggaagtgacaggaaagccacaccatctgccccagatgctgtcaagaggcgaggaagaaggagctgacagagtgcccaaagggaacagtgtgagcaacatcaacctcttccttcactgctgtccatgaggccagcccgtggaactacactgctgtggctggagccacaggattaagtgacctctactgaaagcctgctacgtgctgagcactgagattcgaatgaggactaagaccaggtcccacactgcaccaggtccacacccaggaacctagccctggtagacattgtagaaagatagatcaaatgtgacagcaacaagacaactctttcaaaatcatcattcacaatttctgatattaaatgctttttttgctattgctgtatttttttaggagcagaatgggagatagatggaaaaatgaagttaagggaaagctctggtttcattagttgaggttacttgaggttttcctggaacttggattgaagatggcaaccaagtgttttcaactgtttaaaatatgatttaggccagatttactaaaacagtttcacaggaattttaaaatgttgaaaggaatttctaaggaagctataatctaatccaaagtttagtctaattcccaggtcaactggaaacttaatatgcctagtttactcaaacatcaacttttagataatttatcatttttagcaagaaagataaagtctaactcaggcaaggctttctctttctgagttaaatggtgtagaatatataatggatttctgcctctgcctaattattcaaggataagtcttgtgatctaaaaaggaacaacgtgctgttgtatccacggtaaggggattaacggtcttgtgcttgtaaaacagcagcccagcgcctatttagtccttttgatttccagaaacaagagccagattcttaccactctttggaggcagttggccctgtctggcgccattgatgaaaaatggcagtggatccaattcttagagaaaccttcagaccagggctttccacacttgaggaaaaggagtaaacatttttacctctctttcatgatttgctgaaagaagttacttatgtatgaattaatataatttcttggtaaaaacaattggtcaaaatgattattaaagtgaattcaaataattccaccagcccactggctgctctgaaaagccatatttgcattgtttttgggtttggcttccagcttgctgcagctacacctgcccttctcggactctgcattattgccagtccccaaactctcggtttctttttagtccactgcattggtcactggtgtgccccaccatgtcagatgcaactgtggacaccagctccaagatcatcaccaaagtcttaaaggagaagaaagaagttgtggaggaggcagagaagggaagagatggtcctggtaatgggaacgctaatgaggaaaatgggaaacaggaggctgacaatgaggtagaagaagaataggaagaatatggggaggaaaaagaggaggaagaaggtgatggtgaggaagaggatgaagatgaagatgtggcagctgaggatgctatgggcaaatgggcagttgaagatgctgaggatgacaacgttgataccaagaagcagaagactgatgaggttgactagacagcaaaaaaggaaaagttaaattaaaaaaaagaaaaaaagagaaaaggccaccgtgatccattcactcttcaccctgtttcagaatctaagtgtagtcatcttgaagtagagaggcctgccgggcaagcactcttcaacacccaacccaaaccacaagaatttgcaacaggggaggaaaaaaaaccccaaacttccaaagctcagcttttttccttaaaagtacttcaagaaaaaaatttgttcatattttttatttacattgtttatttatttgtttttttttgagatggagtctcgctctgtcacccagggtggagtgcagtggcgccatcttggctcactgcaacttccatctcctcggttaacgcaattctcctgcctcagtttcccgagtagcaaggattacaggtgcctgccacaacgcctggctaatttttgtatttttagtagagacggggtttcaccatgttggccaggctgtctcgaactcctgacctcaggtgatctgcccgcctcagcctcgagagtcctgggattacaggcgtgagccactgtacctggcaatattttttattttcattgtatatttttgtacatattgttaggggtcagccatttttaatgatttcaaatgatcaaaccagccttcagagctttctctgtcttacttctgactttacttgtggtgtgaccatgttcatgatacagttcaaagaaaaaacaaaccttgaagaaaagcaaaaacaacaacaacaacaacaaaatcttattccgagtgttccagtgtcttttttgtatatgaacttagcggtactataagtttgtatgagatggttaaaaaggccaaagataaaaggtttctcactcccctccctgccttttttttttttttgtctgtaaagttgctgtttatttttttggcctctttgacatatgtgtgaaacaatatgatgaacaataaattagaattttgtttcgctgagttgctctaacaacaacaaaaaataattccattggactcattcatatttaaaatcttgaaaatgtaggatttttatttagttgtgctaatggcatttcagtttctttatgacatcaaaacgtgctttaaaaagacatgtttagctcaagaaatgcttgttgatcaatttttcattctgtacaaatcttggaggacattcaatgtagtcacatttggaagaaaggccatggagaagtcacattagttgcacataatctataactggaagaaatttgtatcctttggttccttaatacataaattacagaactatgtgcagtcattcttcatgttacttcctttttagcaccaactttctgccttcacatttacagttcttcttggctactttcatctacagtatattcaggattcttcttttgccagagagttctgtgttttgttttgttgtctgagtatttgtaggtgagtagataaagtttcgggaaagtaatctccttccctccctgcctctcttttctgttattacagccctggaccaatttacttcctcagtttgtctctctgggaataatgtcagaagcactgagatgttaaccttctttcatccatccatttttccccccaatcacttgctgaatccacgtgggccaagcttcaggaatatgggggcttacagcacaggacagacttgggactcatgtcctcatgtcccagggcacaagagaaatctgctgcaaatggacaaaatatttgataatctccctagtgaccctgcaactgatcaaattaaaacaggtgctctttgcttggagatcatgaagttcaaatcagggcagggattagagaccctcctgacaaagaatgcagacaagccatgtgaaatctagaagatctttgtcctccccatgtcctgggtctattctcacatagggcatggaactatgtgcaaattctgccaccgtgggggttactggcttccactttggtatttaatgctctggtggactcaagactctggtcttggttttcaagagtcttagcatcactaaattcctgcatgggatggggcacggaatctgaaggccgctgtagtaggaactcttttcctaaccatatccctaaccatactggctaatcaaggttagctcttagtaaatagttgtgagtgaatgaagcatgtttaacatcctggctcttaaaccagaggcctgtctattgaggtgcagtggacacaagaaaaccatgtccccccatccccccaatttctttctctttttcacctcagtcccacaggaggatgctcccatggtggatttggcacacaatggccagagtagaagcagttccctgttgcctgggtattagctcttgttggacctcggtttcttactcttggggaggccaagacatcagttagtttctgaaagcatcatccaagcaagtaggaagtatcagcccagagtgtgtctccatcttcatatacagcctttgctgccttcagagcagggctaggctttcccctggcccccttcccacaacattgctactgtgggaagtgggcttattaattcttagttcaactgcctgtatgtgaacaaaatataaacacgtctgtagagttatatatctgttggtgacatttgttgaacattaggttacctttttgtttgcttagaatcttgagacgagttacatatttgggcagaaacatcatgtaggaaggacacaacattgtcctgttgaagaagaccacaaacagccaaaactatttacctcttctctttatatcaatcttagagaaatgatgcttaagaaactagtacgatggaagtcatcataataaataattttacatctgaatgtataacaaagaatttttaaaaaattcaggtaactattaataccatccttatgctggtgggtaccagtatcttgcatttgaatacagctttcttaatttcaaacaaattttttaaatctgttatctccttatcaatgaagctagtcaaaagcattaaaacgttgcctagtttttatttttcgtgtagatacttttactgtgcccaggcaactttttaaataactgctttgctcttatatgtatgtatatgtctgtgtatttaattaaaaagatttacaaaagagttacaaagttaatatagagagtccctacgtaccagtggttctcaacacttggtgggggggttccctgcaattgctcccaaggatatctggcaatttctggagacattttggttttcacaattggatgtgctactggcatctagtatgtagaggctagggatgctgctatatatcatacaaaacatagacagtccccgacaacctagaattatccaggctaaaatgttaatggtgctattgagaaaccctgctctaaccccaattcccttatgatatcatgttaaataaccacggtagacttatccaaacaaagaaattcacgttggttcactttttctactaatgtcctttttctgtttaagaatctatttctccttagtctgatccactctatgacactttctctgtcttcctttgttttcatgaccttgaaacttttgaagggatttagaagtgtttcttacttggtactgaccgccccctgcccatggagtataaggaaatgggatctgtatgggggttatatctatgactgtagagtgctgtggcatttaaagggtggggaagagagataatcagtgtggtgaatggctcaagacagttacccataacattcccacccaaaatgccaacagcacacccccattgacacactaagagctcaagcccttcctagtaactcatagacagcgaaatgacttgtctcaagaccaccatttggcaggtagtaaatttcaaaataaaaactttgtttttagagtttcagtccaggaactttttctatctaacaataactgtctctattgaaaatgtaaggtttactattttcagcaaaaattttaatcttttggaaatctttatttctaagtagataaaatattttcattttatttgctgaggtaatgtgttaaagtatgagttgccacccattggaaacttgaataaatttaaagaacttgaattcatggtatagttccatagtgatggggacagaggaaagtatttggggccatagttgtgttgatggagttgtatgcccttccttttatttttttttctttatagtacttacttcacacctccacagtgtatgcatttatttacttgtttatcttttgtctccacctccttagcaagctccatgagataaaggattttgctttgtttacagctgtattctatggctctgtaatggggaataccatgaaattcatgtttgaacagtatttaaagaggctttatatggaaaaaataatacaggcaaagtaactaaatgactctttacatgatttctggccttttatgcctgaaattctatttggtatttgtttaatttatataaatgttatgtactacgcataatattttacctcttatctttttcacttgatggtgtatttttgagatttattctttttttttttgagacggagtcttgtttgttgcccaggctgcagtgcagtggcgcgatctcagctcactgcaacctccacctcccgggttcaagcgattctcctgcctcagcctcctgagtagctgggattacacgtgtgcgtcaccatgcctagctaatttttgtatttttcgtagagatggggtttcaccatgctggccagactggtctcaaactcctgacctcgtgatctgcccccctcagcctcccaaagtgctgggattacaggcgtgagctaccgtgccaggccaagatttattcttttatttaaaaaattatatagctctagctcaatggttgccaccaggggggtgattttgtccttcaggggatatttgggaacacctgtagccatttttgattgtcacaactggaggtagtcctgatacctggtagatagagctcagatttgctgctaaacaacctggaaagcacatggtatcctcacaacataaaattatctggcctgatcgtactgatgttgagaaaccctgggctatttctttcctttaatctgtggaacagtattccctcgacagcatatactatatttactttagtcccgtactgattgacatttgggttgttccaattctctgctatggtgacgagtgccgcaaagaacatcctcaccagtgtctgtgtatgtgtgccacagggtttctggtagctgcgttttctgttctgggaatcacttccaaatgccattccagagtggctggactccttgcttccacagcgtcacctgggattccaattccatgcgtctttaccagcccttggtattatttaacttttaaattgttgccaatttgaaacgtttaaaatagtagctcatttgtaacaattttgcatttcctgattgctagtaagaccagaccacaggaatcattcacaccccaagcattctagaaaactggtgaaaatgttttctgtccagcatgcgccacttcatttggtttgtccctacgtctttgatatgtattagaaaaccgcacattttgggggactaggcttgcctgtttctgccagtagaggtcactgttcctcagtggttctccaccagtatgacttttgacctctctaccctcccagggagtatttggcaacgcctggagccatttttgataggactgtgagtgtgctgctggcacctagaagtgagaggccagggatgttgctaaggccctgcaatgcacaagacagcttcccgctcctccctcccccttcccctcccccctcccccccccaccaagaaagaaccatctggcccaagtagtagtgccaagctggagaaaccatggtgtcgtctttgaaagaaagaatatgacaatctctatgataataaagacacctattattattaacaacaaatattttattgcggaaactggggagaagcatgcctcctgtccccagggagaaggcaggagcgaatgggcaagggcaagggaagggctttaagggagaggagaggtgggcctggggctcgctgagtggtcggctggagcagtgggaagtgccccggtggggccggtggctggtgtgggggtagaaccgcggtcagtagggtgaaggtgctgagggctggaggctcaggaggtggtggcaggggcctgactcttggagccgggcagcaggctgggtgtgctcagaggctcatggcccacctggcgaagctccttccaggtgcaggcaagcctgaggcgtcagggcagtgctacccgcaggcctagcgctcctgcggcgaccgggcgcctggcggccacatatcctcagcagggcaggcggccgccaggaaggattgagacaacgctgcccgggagtaggggctctgctcagcggcctagacccgggcagaaaggaatgggccactccgtttctcatcacagacccgaaggcagatggcctgggcatcgggctccctctgcgctcgggcggggcgattttcatggttaccaggctgcggacaggctggtgggggggtggcggcggtccaaagccaccgcttcatgtaggccaggcgagcctcagagaaatcaggcctgatgttgctcagcagcggcagcctgttgggaaagcacctccaggcctcgggaaccatctctctcaggacgcggatgcgggcgggcaccatctctctccggatgcggatgcgggcgtagtcccagctgggaggagcggtggggaggggctggatgcagacccagcgcttcccgtggaagtaggtgaccttgcagttctcctggacctgggagctgaggaagggcctggacagtagggcccagtgcttctggcgcgggggtcgggcgggggggtgggtgcgcggttttctggcccagagccagcccaggaagccgctcaggtagctgcccatgagagcagggcagtgggttgggccttccagtagacactgggctgccagaagtttccgaaatctgcggtcagggccagagatagtgtccgggctcagtgaccagcgacctttggtatcaaaatgtcagaaagtgcacgaggcgccatgtgccgggtacggtctggccaccaagtgttgcatggggcaggaggccacggccaggccagagctttgcggcgcgcagctgcagaaggggcaagtggcgccccatgctgtctggcggaccaaggaggctcaagcctcagtccttcccactcatggtcccagacactcagctctgggcagaggggcctggcagctgcctctacttttgtacctgacctttaaaaacagaacatagaagaatttctgccaggtcctgaaaactgcagatacctgttgacagaagggtgctctctttgatcagtgtgaaaatgatcacaatacacttggatgctctaaacaaatcctttactcatatcagattcttctaggatggatgttaatgttgctttttgcaattttgctcttaagtaaactgggaaagccatcaggttttgttttagcattggcaatgttgattttatgtggactttcctataatttccatcctgagtacatccatgtatagattgtctctctctgaacactattttcatttctcccctttcacttgtaatacagaggagagtgagtcacatttccaatttgcagttgaaaagtctgtaactgcaaaacaaagagttcaaagactgctggtgaattgattatagtgtttaagttggaggtttgttcaaggcgatgtaaaagaaacttgtttttgagaactttttcttcaaaagtacacacagaaggtgccttttgtcttggccctgatgctggggtggaagagagacagtagatgcagacagagcaagtattgaatcatgagggggcagcagatcctctctggatctgagaggtggaagactgaatgggcagggcactaaaaagagttgaatgagaaaataccaaaggtttctgtggttcagagagggccaagcattggggaaacgggggctgtcaccaaccggtgatggagaatgagatccctaatgctctccactgactattattattttttggcagattccctgttattccctataaggaatgatccccaagtatatgattttgaaacaatgggaatttcctatataattttcttcttactgagctcattcttcataatggtagaaatgactttttaaacatagctaagatgtctcttaagatagtagtagcatcagagtgaacggtttaatgccatttggtatgatattcttctcatgtattttttctttccactgtctgcagaatttcccatgtcctactttgcaatgtaacccagagggtatcattctggtttgtggttacagacccttcaaaaaatcacacccttcctgctgttgaggtgcaatcagccataaggtaatcacccttaaatatgccttctattagtcaatcaacttgctgattaattcaactgtttttaaaaatagatattgaagagttttatctgtttcatacatgaattatttattaaaatgacaaagatttcattttgttagtttctgttagagatgagtatagatcagtagttctcatccaggggcaattttaccttcaaggagacatttggcaatatctggagacatttcgcaatgtctggagacatttttattgtcacagctggagggggtggtggtaccaccagcattgagtaagcagaagttagggacccaatagcttacctttttcagtcaatatatagtctttactttaaaaagataattttttttaaaaaaaggtaaaatcatgactctcatgcaaactaatcattattactatcaaaaattttatctcattagttaatgagggcactagtaagatgttaaattctttttcaaggagacttcaaggaaccacccatatatagaaagtgaggaatggcaaaatgcatttacagaaggatgcatcagtctatttttcgggcagtaaccaattatgttccactagacacagttcatttgattttctatggacaagaatcatttacattatgttagttttcaagaacttagggatttgcaaagtagctcaaagaccgtaagagatggaaattgcctagaaaggtgctctcagcaggacaatcaggaaaaagtccagtactctgttttgcccatttcaagtattttacaatttttcccgacttcccgacagttgtactcctcccctccttatgatcgtaagaaactcaaaagtgaaacgaattcgagactttcaagaattaggaggatgcaacttgtatttctttcaattactgtgcctttggtatgtgtcaaaacttcaaataccacatgtgattgatgttgggcaaagatatactcaaaaaatgagctcaggaaataaactagaacaaagcaaagtagctctccccacacaagatttacaagttactgaaatacatattattctttttaccaaggataaatacctactggaataactcattagctctctgtaaaatgttaacacttaagataatgagtatataatcaaatgtcaaaggtttttgtgaacatcatctatttcttttgctgaagcaaaggcactgaggatttatactgattggaaatggttgaatctgccctaacaagaaagtttgaactaaattgcttcttcactgtggccactaagaatactgttaagactctcaccacttagggagtaagaattataaatatcatatttcatgtgaaaaaagattttattcatgtcttgtgatctgtgagggctacagggaaaatgaaggcactgggttaaaccagtccttcagttgataggaggaggaagagtgagtgcttagggtgaaggtctgggaagtattaatacatgaatatgttcacaacaaaatgtctttttcaagggtcctagcaggagaatctatttaggaagtggggatagccaatagaatgagaatttatgtcagggaattagtagggcacatcaggattggatgaaaataaaggaaactatacaacaaaggaaggatagagtgaaaaaaaggagatggtgtttagagagcaaataataggaatttcaaggctgaagaatctaagtgattgggcaaaataatactagttccacctaatatttgcataccactttttggttcacaaggaatgttcttatgcatctgtggcatttaataatgacaccaacagtttttttttgttttctttttaagaggcagagtctggctttgttgcccaggctatagtacagtagcatgataatagctcattgcagccttgaactcctggggttcaagtgatcctcttgtctcagcctcctgagtagctgggactacagggatgtgccactatgcctggctaatttaaatttttttttttttttgtagagacagggtcttactatgttgcccaggctgcccttgaactggcctcaggcgatcctcctgcctcagactcccaaagtgctgggattacaggcatgagccattgcacccagccaacaccaataatttttcagtaattactattatctctcctttataaatgaagaaactgaggctcagggagattaattgatttgttaaggccaccatgctagaaagagaaagtgaggacttgaacctaggcatcctgccaccatatccagtgatttttcctgtttaccagagtacacaaagaactgaaacagacatcttagaattggttggtcattcctttacacaaagtgcttagggtgaaggtccaggaagcacgtgaatttgttcacaaaatgtctatttcaaaggtcctagcaggataaactatttaggaagtggggatagccaacagaatgagaattacaaaattctcatttcaagtaaaataattgaaatgggcaaaaaagagtactggactttttaccaatacagagtatacatagtatgaggtaaaggagtgaccaactaattccaagatgtctgttttagttcttttgtgagggcgtggcctaattcaataccactcttggctgaaagacatacatgatgcttgatgccaattggtgattccagtggtggagcagatttaatatgtacctgagctagaacttccagtcagtctgtgagatgatagaatacattccaagcctagagtgagcaagtaacaaagggttaaccaactaggaagatagaatgagaatgcagagcaacttctcatttttctctgagttatatcttagcagtgggtttcattattttgtggaaaagtttgtgctctcagatgttcaatattaggttatgacaaaataaaatattccattgttggataaaaatcccttgttgtagattatctggctagagttcactttcacatccatgaataatcatgccacagcatagttatttatttactaaagggggtctttcctgatagcctggtgctggcctgcagagattctcaagccatactatcaggctgtagacttggactgtccggtaccgtagccaccagccaaatgtggttattaagcacctgaaatgtggctagttcaaagtcaggtgtgctcacagtgtaaaacataagccaaatttcaaagacattgtacaaaataaaatgtggagccccagcctgggcaatatagcaagaccctgtttacacacacacacacacacacacacacacacacacacacacagccaggtgtggtggcacacacctgtggtcctagctacttgggaggttgatgcaggaggattgcttgagcccaggagtttgaggctgcaatgaaccatgatcgttcctgtctcaaaaaacaaacaaacaaacaaacatgcaaagtcaacttgatactgtttacatgttgaaattacattttcttggatatattggacaaaaaatacattattaaaattagtttcatctgtttattttagtgcagctactagaaaattttaagtcacacgaagctcacattatatttctgttttccagggctgctctagaggtagacaaaggcagcttgcataatatgaattcagtaaaggtcataaaatatctccaaggactctccagtttacagttagaaattcacttatttggtttcaattaaataagtctttgctaccaacctctccttaactgccaccaccactaaaaatgcagatatttcctaggaaaagtaacatttttctaagcttttgaactcctaaatcattaatgtctttagtcctatggaactacttgcaatttgctgaacaagtcttactttaccttgtttccattcctgtgctcaccacatactttggtctaaaaactcctacttgtccctcaaaacagagctcaaaaatggcctccacagcaaagcagctctgacttctcacaatggtgtacttggcccttccctctgctcacactgtgcccatatttgcctctttcacagaacattttgtaccacactgtggttgtggatctactgatctgtcacccttctagaccgtaagctccttggtactttaatttcacgttgtatctccagcatctagcacagtatttgatatttaataagtttttggcaaatgtttgttgaattaatgaaaaaaatatcaagatcaagttggtgaatttgaagccactcctggttcatgttttcatttttgcttctggttttctttacttctcctattcatcacccatattttacatgtaattttacttttgggggctgtgatatttatgtaagccaccttaaatagagctttgtcattctaatctagatgctactcacacaggacaggtgaaacccaaattgaggcttagcccatgaggattctgggctttgcccaggaaaaaaattcaagagagagccagaagtagaagaaaacaactttactgaagaggcagtattatagctcctgtgatgttactgctctgtgactgctcctgcagagcaggactaccctgtaggcagagactagcagctcagggcagttttgcagtcatatttatacccacttttaattacatgtagattaaggagcagtttatgcagaaatttctagggaagggtaataactttggggtcatcacatcattgctatggaaaggggtggtaactcccaggtgttgccatggcaatggtaaactgacatggcacactggtgggcatgtctgatggaaagctgcttccccgccaccccctagctctgttttagttagtcctcagtttggtccagggttagagccctgcctccagagacgagtccctccgtctaccttactacctctcaggaaataaaacatggtggacagagctcaaagctcaggacttctggacattcacaacaatgggggcagcagtggatacatgggtatccctctggcagctagtgttgtaacttaagagaaactatggcatgattgccctcagcaaactttttagtcaacttgaagtctcctgccaggaggatgctttgtttatagagatatttataatcatggagaaacaggtttgtactttgtcttaatagttttttttctgtcttattttgtttatttttaattatcattgcattttagaatgaacaagaaccggatcaacaatgccttctttctaaatgaccaaactctggaatttttaaaaatcccttccacacttgcaccacccatggacccatctgtgcccatctggattattatatttggtgtgatattttgcatcatcatagttgcaattgcactactgattttatcagggatctggcaacgtagaaggtaagatatttaaaaggagtgattgaaagagcattttcttatttcctcatgacttggacaaatcaggagaggattttcctctggctgaagataatgccttaaatttgaaactttgaaaaaattggtgattttcaaaaatatcacttatgctcttttctttagatatattttagagtccagcgtcccttacttaactaggctccttccacgactacctttccctctattctgtaccacataaatgtttcatatccaggcttgattttaatatctgtgttgtgattttgtaagctagatgtaaggagctaaacagactttttgggtaggcagagttaatctttggtagtcatccacatccaaatgccagcaggttcatagttgcatgcctccctactttggcttacctcttcttagtccagtgcatgcaggctaccatcttgtcccagatgcctttgtactgttttatacttccttccctttgatggacactcatcccttgtgtgcctatgatattgaatagagctgtgttgcttgtggcctttaaatcaactctcagcatcagacatcacctggaacacttgtttaacatgtaggttctttggccctattgcagacccactaaattagaatctctgaagggatgcattttaatgaatactcaggtcatccttattcattgagtttgagagctttcaatttaacaatttataggtagagtttcacttcttgagttcgtttacctagattctggaggcatttgcaaacttcgatgtctaagttattgatactgcttcaagagggaatttaagacctgtggggaagttactgaggtagtggagaagaagaggagacaggatttctcagcatgattttgttaaccagtacctctaatgcaggggggaagatggaaaaatcctcagcttttcactagactttaatgcttgtaaaccttcctttctgatactaaggaagaataattctggtaagtggtaattacagtgacatgcatttgaaaataacagggacatggtttttaaaagtgttggttcttacatgggacatgtgggatgtgtttggctaagtaagtatgtgttgactaaatgctcctcttgaagtttactactttccatatgtttcaccctagtaactaacagaaggtaataattttaaatcttgggctttgtaattttattcacaaatgataagtaaaatgctatgaaataaatatggtttatgtctagtcctcaaattaaattcaaagatttaggatcaagttacacttacctgatttttttgatgatcttggcatggcatgtaatgcaaagagacttggtacaagatgtgcattttggtttttcttttgaaaatattgagaaatattctcatgcaatgcttgaatacctgtgtttaatacttttgttttgtattaatcttgagatgctttgattctgtgttctaacaactgtaacattagtgaattcactccatctacttggtttctaatttcctcactagtacagtatagtgtaggagttagaagaatggactctggagatagattgcttagactcaacatatttatctcttagattctgcccttgtatataatccattaactgtatggatatcaggaagcacagtgagtcagggaaactgagcttaggagaatataatttgtggtgctagactcaagctggttaagtttatctttactgtttgtgaatagataacttttctagagtgacagtgaggtgtactggaaataattaacttggtgggataaaaagagttaatacatagagtgcttagagtggtatctggcacacagtgagtgctcaataagtgttagctgttactattattttatgatcttgccatttgctggcctttcagctagtttgtattgggatcagctatctctttccttctgagcagattctgcagtaatataataattgggtgtgtagcatcataatttgttgcctgggagaagtgaagctgttgtcaccaccttgcctttacaaatttatttcatgttcaaactgggaaagcagatggacacatctcttgtgtaaaccgggagagcatcaataattagcaaatatagtcaggaaaattatgacaaccttctgaagataaattgtttgttgtgttttaagtttgacacaatttgtgatgggtttctcctggacaaaatgttgcgtcaaatgtgaggagagggtgcagtttgaagtggggagagccctgaaaattgcaatgaaagccctttctaatcatggaaaagataggagttgcccccaaaacctttagtttcgaggaagacagtcaaaggaatgttcaacaatctaatccatacattagataataatgaacatagtttttgtggtacagaatcttaaacttagaagacctggagggaccatgggattatctaagtcaccattctgcttctatggagaagagtgccagagtcatctaacacacatatatatatttaaacttactattgaggaaatttctcagcaaagctttaacaattctgttatctttgccaataacaccattatattaattattcagggacagtgagtggcaagtatgcaaccaatggagcccaaactaggtaaactcaaataccagctgtgccctttacctgtataatcttagttgagtcacctgatttccagggatctcagtttctccatctgcaacagggagatgatgtttcacagggctttcatgaggattgactgacataaatgtgtataaagtacctaggatatagtgtgcactaaataaacagtaattatgattatccaagatcggcttaagccattgctatgattaagaggtgctgggaaaggatctcttttccaccaagatattgtggaaaatatcttggtattttccatcatactgtagaaacttctccaattcaccaactcattgcttgggattttattaaatgcttgttttattttattaaggtgttgttaacaaataaaagttgtatatatttatggcatataaggtgatgttttgatatatgtatatattatgaaatggctaaatcaaataattaacatataaattcaacttatttatgtggcatttgtctttctgtgcctggcttatttcatttagcatactgttcttcaggttgatccttattgtcacaaatgtcaggatttccttctttttaaaggctgaatagtatttcattgtaccacattttctttatccattcattcattgatggacacttaggttgattccataccttggctgttgtgagtagtgctgggttgaacataaaagtgcagaaatctctttgacataatggtttcaattcttttgggtatatacccagagtgggattgctggatcatatggaagtttattttttattttttgaggaacttccatactgttttccataatggctgtactaatttacatttcctgcagtagtgtaccagggttcccttttctccacatcctcaccaacacttgctatgtttcgtctttttgataataggtattctaataggcgtgaggtggtatctcattgtggttttgatttgcatttccctgatgattagtgaagttgagcattctttcatgtacccgttgggcatttgtatgtcttcttttgaaaattttctattgacatcctttccccattttaaaattgggttatttgttttcttgttattgagttgtttgagtttcttatatatttttggatattaactcctttgctgtgcagaaacgtttcattttgatgtaaatcccatttgtctatttttgaatttgtttttgtgcttttggggtcattgcccagaccaatgtcatggagcttttctcctatgttttcttctagtagttttatagtttcaggtcttaggtttaagtctttaatctattttgagttgatttttgtatttggtgtgagacaacaatccagtttcattcttttgcatcgagatatccagttttcctaacaccttttattgaagagattgttctttcctcattgtgtgttcttgccatctttgttgaagatctgttgaccataagtgcatggatttatttctgtccttgctattctgttccatttgtctatgtgtctgttttgatgccactaccatgctgaataaacactttttaaaaatgcaaataattctatgaggtctactggtcaccacaatagtggtgaattggttaaccagctctctaaaaaataccctgatttgtagcatttgccagtttcccatcaaggctgaaaaattctgaataaataccctgatttgtagcatttgccagtttcccatcaaggctgaaaaattctgaataaataccctaatttgtagcatttgccaatttcccatcaaggctgaaaaattctgaatatttagcaatcagcccttgaaaacctgtaccagccagatctagcacaccattacagctgccacctctttgtgaaatacctagaagaggttgcttctctattgccctctagatgagaatagcagtttgtgaaaatgagagatttgttgaaggaaagaagttggaaaagcgattttttttcttttatttatttatttatttatttattttttattattattataatttaagttttagggtacatgtgcacattgtgcaggttagttacatatgtatacatgtgccatgctggtgcgctgcacccactaactcgtcatctagcattaggtatatctcccaatgctatccctcccccctccccccaccccacaacagtccccagagtgtgatattccccttcctgtgtccatgtgatctcattgttcagttcccacctatgagtgagaatatgcggtgtttgattttttgttcttgcgatagtttactgagaatgattttgaaaatttggggcaacaattagggagcagaacaagtgggaaggcaaccaggatgaactgcatatacttcacaataatgtctctactagatttagtgtctagccacaggctagaagtaggtgtgttagttgtatggtcaattttatgatggtagtgataatgatgatggtgatagtgatggtgatggtgatgatggtgatggtgatggtgatgaagttgataatgtgatgtgtgttttaatagaaagagccctaaactaagactctttgttttgggttccagtctcaggctctggcatttgttagctgtgtccttgagcaggtcacataaggaaaatgatgataatattttcttaaatagtgtaatgatgagaagacaaaatcagagaatatatctagtggtttacatgctagaaagtgctatatcaatttaagttactagcatgaccataaaaagcagttataaagtgctaattttatataacctggaactttgtagtttacaaggacagtcatggacacagcacaatttcagatttacatgtatatactagaattgatgttgaatccagtatataaatagactgttataatgttagaccttagttgtatctaagatcacaactgggtcttaggaagggaccttaacaatgatccaaatccaaccctccagaaaccaaagcccagataagtaagaatattttattgttttattgaccatccattctgtgataggagctatagtatgtattgtggatgagacaaacataaataacatggatggtccttgcctaaaaggagattgtaatcttatagtaacaaagccaggtcaaagacttaggacatagaaccctagctaatgctcccagctgagtagagtcattagtaaatttgcttggtttgtttctagttatacttatattcccataaggtcaatagtaaataaaaatgcatttggatttccacagatcttgtttttcacacaagcggatgtggctcagtcaacacttttatatcctgaaaccacattcttataaagtcttaattaaatgaatagtcttaaaaatagaggtgtaaataaaatgagtaatgtcatagcgaatataacaaatgtttttctttgctgttcttaaatatacatacctgataaaaatctaatgtaagtgtgttgagtaaagcctggggagagtaaatagagttctaggtcaactcctttcaccagtcttgtttcattctgaaagacaagttactctacgtatttgagtgtcaattccttcatctgaaaaacagagatgaggcacgtagtcgtggctgagtggttaaaacgatggatttgaaaaacagtgataataaaatatgcataatattgcagtaagaattaagtgagatgatgcatacaaaacatctagtctgatttctaccatgtaataaatacttaatattatcattagcattatcattgctattatatggcacatgtaaagcaacttgcctacagtcacatggttagaaagtaatagtaacaggatttaaaaccaggtaggtttgatttcaaagtccatcaatctcactgtgctacagtgcccactcaaatcattccagtaaccctatttacccccagaatcatccagagcaattctgaggatggtctcaattccccgataattgattctgtactgtttgaaaatcatatttgtctgtcttttattgtctttctggctattcttgggtcttggctctttagctctatttttttttttttttttttttttgagacagagtcttgctctgtcacccaggctggagtgcagtggcaagatcttggctcactgcaagctccgcctcccgggttcacaccattctcctgcctcggcctcccgagtagctgggactacaggcgcccgccaccacacccggctaatttttttgtatttttaatagtttcatcatgtcagccagggtggtcttgatctcctgaccttgtgatctgcccacctcagcctcccaaagtgctgggattacaggcgcgagccaccacacccagcctctttagctctatttatttatttatttattttgagacggagtctcactccgttgccaaggctggagtgcattggtgcgaactctgctcactgcaacctccacctcccgggttcaagcaattctgttgcctcagcctcccgagtagctgggactacaggcatgcgccaccacgcccggctaatttttatatttttagtagagatggggtttcaccatattggccagcctggcctcgaactcctgacctcaggtgatccgcccgtctcagccttccaaagtgctgggattacaggcgtgagccaccacgcccggcagtcagctctattttgtttgaagggggacagaaatgctcttcctgcttttggcaccattactttatcttagaacagagcaaaccccgttcctcaacatcccctagctctgagggccacacctgatacttctttcctcccctcttttgcccctcttctctctatatttatgaggtatatgagagatattcactctcaaggcttctaggagctcgttccaggaattttaatcagttttttcatcctcttcctgaaccccctgacattgcaggaatagcagaagtaattaacacttgattttagttttcaggaaaaagaactcatgtttttagcagaacatcaaatcagaatgatccagaaagatagttaagttttattattcttagtaaaagcaatgcacaggtagtgaatcaatttattacagcaaacactatgaaatgtgacaaagtgatatattactgttagatagcctttgagctgcttatcatgtccaccaagcagcaagttcctgacactgcaattggtgaagcccagcaatgccaggactctgcaactggatctatggttgtaaaagaacaatatgcacctaatttgccactgacacagctcagtttgataaatttctacagcaattccccttggaatctttttggttgagatattattcttattctaaagaagaaaaacttgtcctcatttgtttacatcttcctttttagtttttccccattctttcgtgatgcctgatgaatcatttttccttgttgctgggaagggggaagcgagattccttgtaatgatcatcacatacgtctagctctttgttatttttcttctaactccataaatactttttttattaactgaagaaactggaaaataggccctggcaattattatttctaagactgctattgcattgccacctacttcagaatgcttcctttactaaaatgtttgtaatacaggtttcatacttaacatatacaaatatcagggtaaaatttggctgtccacaatctatttattgttgtggaaaattatcattcactccaagcccagtgaggagtgattttgggggaatttacatgagatagccattttaaatgatgatttataaatagacatttgctacttacagcttcatctttagaagaaacattcctctctcttataaagcattaaaatggggagagggaggatgagtatatatggcatttttccctttcattgagttcattttaggacaaggactgtatctcttccatacacatagtagacgctcaaaatgtagctgttagtgaatgttggatactatgaatggaagtgatgtgattgatggttatagaatgttgatttcctttgctttctggaggacgtggaaagtgaacaatacacctatctataggaagaggaaccaaagtgttctaatcctctggtgggtaatttcacctggaaggatcctctcatcacattaaaatctatgtccaccccaacccatgctgattgctgattgctgatgcttgccatgaccagtgtggtgtcttcactccaaggatggaaaaatatagcagcctatctttccctaggtctctaggaccacaactcctgactttcaacccagcattgggccagaagttttattaataaatgtttaaaatgtggcatctgcaattcagtgattgcctattgtgtttaattgtgatatttgatggtggcatatgcaatggtaaaatactttcaaatgatgacattgggatctacagaattggaatgctactttacctttgttttaaatgaacaactatttgaaattatttaataaaccatcagcaaagcctaaaatacaagagatacaagaaatttcttgtgtgtaaaattcccttccttccttccttccttccttccttccttccttccttccttccttccttctttcttgtggtaaagcccgttcaaggtctacctagggctactgcctgagtaactttacctacaacattagtccatacatacatacatctaattgttcaatctaccatctatcttctgtccatctatccatccatccatccatccattcagccagccagccattaatatatcatctagccaaccagcaattgaactacattcattgattattttatccatctgtccatccacctttcatcatttgttcagccacccacccatcttctgtccatccatgtatttattatccagccagtcagccattaatctacattcattgattcatctacccatacagttgaatgcctactctgtgctgagtccaggtcagctgttttgcaaaacatttttaaatttggatttttctgattaggctccttattatgagattcaggtcaaaacaaattggagggggcggggctggaacacaggaaaaacatatatgtgatctactcagtgtatcacatcaggagagacttgtcagtttctttcattgttggaaattttcactagatttctcttttagcaaaatacaagtgtccttttataattaatattttgcttatgggggtgatatcttgaaaccatgtgaatatcctgttctccaaccgtctctcacataatggtttagtatccattgataatcctcatcagaatcaattattattatatggtgattttctgttcctattactctttctatattcgttggcattcttctataaaaaaagagctttctcttttcttcactttcttccaccatattctttttttgaaatatatacaatttttatttgtcaattatacctcaataaaactgataggggaaaatgatacagaggacaggaatggagtcaggaaaatcatgtgggtgggaaagatgatttaaaaaaaatttttttttattatactttaagttctagggtacatgtgcaccatatcctttaaagcacattctcacgcaggactaataggaattccatctcggtcatcattggggcaccttcaccatctcctcacctgtcagagactcaccaaggttctaagggactctcatagtggtgggactctacctggtcaccactgcagtgcccaacatctcagccagatgatctattcttttctgctatttggcatttccacaaattcgtgggacaagagaatcctttgcaagtccaggagcctcatgcctaacatcatgcctccgtggtgtcaccatggagtggtgctccaacatccaacctgtgataagccttagggaaacccctccttcttctggttgcctagaggcacccacctctcttttttcttttactgcatgatatcaagcataattgtctcattgttttagtctttcacaaaagacaggaaaaacctttgaatttgagtctactgttttccacactccaaagaaatattacagatctgcatattacttcggttggggtcagcaaggtctacaaagggtgaaactatgagaaactcaagaatttatcctgtcatatggaatttttggagtgacctggaattataactacaagttgtaaagtaattagggaaaatgtattctgagttccaaaatcgtgacttgcaaattaacacaattattttgtaatgagtagagccaagcagcaatatttgaagtttatagcacttactttctacaaagtaatgttaattttctttctgatagctcatttctatttgattttcactcaacattttcagaccattaatttgtcaaatcccatgagaggaatttattacttttagcaattcaattgttattaagttctcctagagatggattttaaacttgcgaatttactatgcattttcttcacattacttcatgcaatttgtgtgtgtgagtgtctatgtgtatatatgtgcatatacactcatattcatatacatattcatctatattgattttagtattctgaataagaccacttgtgttgatcactatgtatatgtcatctatacagacttctcatcttgatagtatcttttcacctcaccaactctttatggtctttgaaattcgtttgaaaattacacaatatcattaaattatttcattgttctaaggttcctattccccttcctgttctccatgttcgttcagaagaccatatgcaaccatggttatggtccttctagatgccaatcactcttttttatgtgcttttagaaagaacaaagaaccatctgaagtggatgacgctgaagataagtgtgaaaacatgatcacaattgaaaatggcatcccctctgatcccctggacatgaagggagggcatattaatgatgccttcatgacagaggatgagaggctcacccctctctgaagggctgttgttctgcttcctcaagaaattaaacatttgtttctgtgtgactgctgagcatcctgaaataccaagagcagatcatatattttgtttcaccattcttcttttgtaataaattttgaatgtgcttgaaagtgaaaagcaatcaattatacccaccaacaccactgaaatcataagctattcacgactcaaaatattctaaaatatttttctgacagtatagtgtataaatgtggtcatgtggtatttgtagttattgatttaagcatttttagaaataagatcaggcatatgtatatattttcacacttcaaagacctaaggaaaaataaattttccagtggagaatacatataatatggtgtagaaatcattgaaaatggatcctttttgacgatcacttatatcactctgtatatgactaagtaaacaaaagtgagaagtaattattgtaaatggatggataaaaatggaattactcatatacagggtggaattttatcctgttatcacaccaacagttgattatatattttctgaatatcagcccctaataggacaattctatttgttgaccatttctacaatttgtaaaagtccaatctgtgctaacttaataaagtaataatcatctctttttgattgtgtgactgattgtggtctggaatttattttcacactgtggaacatagtcttgtttttattagttatctttaatctgctctacaaccatagaaatgaattatttttgttgtgtgtatccttgtttgaagacaccttgtagtttttaggttgattctatttccagataggccatgaataattctaaattctaggaacaatgattatacatttattaatgaaaataaaatttgctactcaatttcccactgctatcactcatagttaaagttacagcagtagtctctaactcacttccctgcctccagtgtgcctccttgcatacaaactctaagataggtagctgcatacagcaggtttattggggcatgcgctcaggagatacacctttaagaagtgaggaaggcaaagtgtggcggacagctgttaaggtggcccgcatcattcccatctcttggtgttcatgccttcatagaatcccctccccttcagtgagtatggtacctgtggcttgcttctaatgaatagaatacaataaaggtgatgagatgtcacactcctgcaattactttatattatatctgagtctgtcttgctagcagatttgtttttgagatcttcttgctgtcttgattcggtaagtggccatgttgggaaagcccatgtgaaagaaactgtggtcagcttctaggacagaggtgtccaatcttttggcttctctgggccaaatttgaagaagaattgtcttgggtcacacaaaatacagtaacactaacaatacctgatgagctaaacaaaattcgccaaaaatgtttacgaatctgtgttgggccacattcaaagctgccctgggctgcaagcggcccacaggccacaggttagacaagcttgctctaggaattgctggtgacctctaggtaccaaggacagcctccaaccaacagctatcaagaagctgggatcctcagtcttacaaggaaatgaattctgacaacaactggaggaagcttggaagcagatttttccccattcaaatctccagatgagaatgcagtctgacctacactttgcagcctgagcagaagaaccagttaggctgtgcccagactcttgaccatggacactgtgagatgataaatgtgtatttttttaagcttttaagtttgtggcagcaacaggtaacaaatagagtaggattaggcagagggagaaacaaccagaaacacggttacaacagaagcctcagccaatctttcaggaagctcaggagctggggctattcctgattgagttaagggagcagggcctttgtatttccacattggccaggcatgcctgtgggtgccttctgggagagggtctatccttgaaaaaggcagttcactgtggctggatgcaattcccattgaaggactcagctgtgagccttcaatagccaatattcttagctgctagggatgggtgtattggcctagaagagggagcccagaagtccccttagaatatctactggtccattgtctgtgatacagcctgaatgaatgccccaaatgtaaacctggtcctgttagttcctaacacagacacttttaaatggcttatcattatccttaggctaaagccagaccccttaacacagggcctgctaaagtttgacaccccctccatctccacctccctccctatcttcatctctcacaagttctctctcttttccatccagtcatacatagatctctcacttcccagcttttgaacaggctgttttctctaccctacctctcagccactcttgtaccttgttcttagggtctttctcacataaacattttgatctcaggaagtctttgccagacatccgggatatgggctgctagaacagccagataattatactcatctcattgtactgtaattattatttttcaatatattttctatagaggaaggggaccacaaaggcaagagtgttttagggcccacaaaagtcctaatgcagtcttgtcttcataacatttttgtaacgcacttcactttgcccttgacctccaactagttagaattaactattgctttgtaatttgattatgtctacacatttttatttataatgattgtaacgcttcattttttatacttgtttatatgtttgtatcttcacactaaatataatactcttagaggtagtgttcaagtcacttagtagagtgccttctctccgtttccttattttctcccttctttctttttttttaaattattattatactttaagttttagggtacatatgcacaacgtgcaggtttgttacatatgtatacatgtgccatgttggtgtgctgcacccattaactcgtcatttagcgttaggtatatctcctaatgctatccctcccccctcccccaccccacaacattccccggtgtgtgatattccccttcctgtgtccatgtgctctaattgttcaattcccacctatgagtgagaacatgcggtgtttggttttttatccttgcgatagtttgctgagaatgatggtttccagcttcatccatgtccctacaaaggacatgaattcatcattttttatggctgcatagtattccatggtgtatatgtgccacattttcttaatccagtctatcattgttggacccttctttctttcctttatcctacaatgttgagcaaaaccaaacacaatccaaacacaatccatgccctcgtgaagctgaaagtctatgtagtcagagctcaataaatgtgtgctaataaaccagcacatgaagtggtagctgttgcaagctcatagttttatcccttctgggagcaaagcctgcccctctctctgtaagtggaagagtttgaatatgtgggtagaagatttttgacttgaacaccattttgacttgggaaccatatggtatttaatattagtaggcttccttatttttagaaaaggaatgagatttgacctgttggttgacaagacgtgacttactaaataattattctctatcttacgctctttcgagtggtgtacttcaggagagtagtttgactttgcatagagaagtctttaactggtgaaacatcagctgaattcaatcagtgtgtgctaggaagcataatccaagatacgcattctcaacagggtgtaaattgttcttggcggggagaaaaaattcgagatattacagtggtttgtagttgtattagtccattctctcactgctgtgaagaaatacctgagactgggtaatttataaagaaaagaggcttaattggctcacagctgtgcaggctataaaggaagcatagccacttctgcttttggggaggcctcaggaagcttccaatcatggtggaagggaaagggggagccaggtgtctcacatggcaggagcaggagaaagagaaaccaagcggggaggtgttgcacacttttaaacaaccagatctcatgataactcactcactctcatgagaacaggaccaagaggatggtgctaaatgattcatgagatatccaccctgatgttccaaccacctcccaccaggctccacctccaacattggagactacaattcaatatgagatttgatgaggacacaggtccagatgatatcagtagtcctccaaatgatcacagtatgtaagcagataaactatatctaagatattaaaatttcatgggataactattaagaaaaaggtctaaaaggccacaataacgaaaagaagattgagaaacactgattctagatgtttcttattttgaaggcatgagacaaacctgttttgataacgagaccattttcttcactatttattctgctgcggagggaatgtattccactgggggaaggtctgttccactaaatatccttattggaaaagttttctgtgctagcagagatgttctgtaaccttgacaatcaatgtaacagcaaaatagagcttatatctcctgcctactgcctttgacaaacactctgggtaattagagagcagtcaatcatcaaccacacctctcacttttaatttaatgatccagtaatgtgccatgagggaacatattgtgaggtgatatgtactggaaacaaaatgttgtaaaggaagtggaaataaagctgtcattccagtttccaagttcctggcaaaaactgcttgatgaaactggaaggacagaaaatcaattcactacggaaagaaaatgcactgggatggtgatatctggatttgcttcacagaagaaatgtaaatgcagataacttttttttttggtgtagtttcatcatctatgtttttcacaagaactaaccattagaaaatataatataaaagaattaagcaagttattgcatatagaatgataatgatcagactccagatacgagtatcactacacagattatgcacatttatcatcattcctggatgagaaatagggaataggagaggacacagattaaaaaccaaaaagcaaaaagaaacccagagcccaaattctcagtccttatttcaattcatggggccccttagcataatattttgtttcccattttacatcttattttcctgtataaaatattctcaagtttagttacataagtaaatgcacattttctttcctttcttcttctcttccttaaaaatgtgttgaatgtcccactactttattgaagacaccatgctagattcagataatatagagaaagaatgtcctataagggacccattcattaattatgataaaattaagttaattactaagaatatcattatttaaaaaaaattttggcttgaatgaatgaataatttttaggtgtcatttactgagtcattctgctctttattttcataacattgttctaaaaggacttttattctgagcacagaataagagccaatttataatacacgacattcatctttaaaaaattattataacacagtaggggcagtagggggatgcaagtagtttgtgcaagtctttatagtggtacaggccagtaggggagagtttggccaataaaaaaatgacaattgtgtctctagatgtaatttttccataattaccccttgatatcacttatattttttgaaaactggtgaatacataaatatgcgtgtgaggtgttggatgaaagaagacatttaatcagatccagagagggtacatttttcaactctctgatgtcttgtgacccaatctggcagtaggatcccagaacaacagagctagggcatacccaagacctttggaaagggtggaacaagaactcttgggcatggcatagagcagaggtgagcgctagccaccacaggcaagcaagcctggaaggtgccattgtccagtgccttgctacagtgaacaggtggcccttatttgcaaaatttagcaaaagtttaattaagattcatgtcttaatggttcttggagtcccctggactccatttgtcatcctaagccattggctcatagcctgccacatttaatgatttatgaagtatttggcaatattcagtttgtctcaaggttgcatttgaaaacttgtgaggtagaactatataaaattactgctatataatcattttgacccacaaaatggcaatgcctcaagctattagtttgctggtcaatcctttggaatttaggaaagatatttccctatataaataggtttatgctgatacagccatcaggccaaatcacagtaacatattatctcacaatatatctgaaatattgcacatctatacataaaatattagaatacaaactgttataatgccagcagtgtaatgaaaacaagcaaaagagaacacaacttaataagaatcagcttttattgtattttgtcagtgcttaaggaaaagcagcgcttttaatttttaatttttttcattctcttctgccacaaatccagatttttatgggaaaacaagaaagtacatggaactagtaaaggaaacttacaggcattctgaagacttttaaagtacattgaccctggttaattaaaaaataggttggcttcaccaccatgcacttgctcctcaactctttgcattctgacttccatttccacatcttgagaaaactgtttcctcaaaggtcactgtgaatttctggttgtcaaggtcacagctttttggtcctcactctcctaaatctgtttgaagcattttacagtgtagggcaccctgtcaccctcttcgacagaatctgccttcttggcttctgggatatggcctcatccttttacttcttctgtctctctcccggaccatttctaaagaagctagggaaaaagttgaaagagaaagaaagaaacaggtgaaaaattatgagtaatattgtgtgaagtgtagttgaactctcagtagtgaaagaaagaacagaattgcattttgtgcataatgaagacaatggaaaaaatacttggggttaatggttctgtctgctgaccaccaatagcttgattgctatccagagaagctttgaagtaagagaaaaaaagtatgtaggtagttatttgaatattaaatgactggttgcttcacatagtgaaaaagagcatgaggaccagaggaaaacatatccacaatcccttatatgcaactctaaaatccaaaaacctctaaaaactatttggtggcaacacttgacctgagcagacataatgctctttattatcttcatttattatgtgtaatggtctttatttattgtagtaaatgataccatcatattactgaatgcagaaatattcaagtgtttggtgataggatgctatcccagatcccactggagtagatgaatatacagcatatgtacagcattaccttgcactgtctgaattctaatcctaacagtaccatttactaactgagtggccccaggtgagcaaggtttcataaaagcggagagaaggcaatccagtgaaagaactagttcccatttgatatcaatagactgtctttagaaaggaaaggaactaatagttgttctatatgcccaaagagttattgtgagagtagaatgaaataatattaaaccataaaattagtaaagaaaatgtgatctgtaagtgaagtttttctttattaaaattgatattgaaattattcaacctgaggccataaagaagtaaggaaagggtttacttaagggaaatactttgctatatgaatatataattgttcctatcatgactaaatatttagaagtaggataacctatccatctggaaaaatgcaaccacacattgatctgctctactagggagcgtaatgtatctatttgaattttcaacacaaagaaattaaacaagacactgcaaaactctactagtatagttgcaaaaaacaaactgcagagagagagagagagcacgtgcaagagagattgaaaagctaattacaccagtattaacttccatccaatcaggtaatgatgaccatgttttaaaatgattacttttttttctgaccatgactatcattgcaccagttgacatttattgaatcaactggcttcccaatcaattcagttaaaaaaaagtttatctgatggatggacaagctattttgttaaattaactagtcaacatggtaaaactttgtggattggtcaaattctaatgctacttagactctcttatactgaggattggtgcctctgacttatctgccaggtatgtgggggatgactcacctgttctatttcttcattgcttggggctcctgtaagcaagaactcaaaggtccttgagaggacagagccataagccagcaggggaacaatttattgctatgacaagccttttggctttcacttgggacaattccaggtgatataatgtgctctttttagagctaaaggaaatttaaaccaatgaatgtaagaactaaaatctgcttgctcaaaagtgatagaggtttctcaatgttaatgtctctgccatcattgcaagagcgatcgaggcccttattaaattcccatcacatattatatttatttactagattatcatctgtacagctataaagggggaataccaaggttcattttagtagtgcaatgattgcagaaagcctcaggttacagttttgaacgcattgctttgacacagcacatttcaatagatcagcaaacaaaacacaatttacaaaacctccccaaggattatgctagagtccttttgtggggagagaggtgggaagttgagggtattgtggggggttagaaatcaatgtacttggcagtttttttcatagtttcatagttctttttatctttgtgctaagaattcaaatagatacctgataaatctacgtattcccttctaccaccttcctgcgagatctatgtgtctttccagatggataggttatctaacttccaaggtttagtcattgaggatatgaatatttatatataaagtaaaatatgtttcctaaataaatcttttttttcctttagtttgtggacttgagtagtttcaatattactttgtattgttcacaggtctcattttattcattaattcttactgtttttcattatttcatttaactctaactacaattagtaatacttgccttatccctttggatggatatcatatctctttggatctccatccaaagggataaggcaagtattactaattgcattcaacaaaagaagaaacagaggctaagcaaatttaaatatgttgcctgaaatcatacagcatttaaaaatgttcataggttgtgttataggattcatagtgattgattgtttttgtttctattttaaattattattgttctatttttaccttatgcccatttaaaaaatactttgtcattgacatgatttggatgcttgtcccttccaaatatcatgttgaagtatgatcctcaatgttggaagtggggcttagtaggaggtgtttgggtcatggggtcagatccctcatgaatggcttggtgccttcccgcatggtagtgaattcacgcaagatctggttattaaaaagagcctggcacctctcctcctcctctttcttgctccctctcttgccatgtgacactcctgctcacccttccccttccaccatttgggtcgggatccaaaaagcttttacttcctgagatcctggccagaagcagatgctgttgccatgcttgcatagtctgcagaaccatgagccaacaaaacctcttttctttataaattaccccgcctcaggtattcctttatagacatacaaaatggactaaggcagtcatcaactcctcttcctggaataacttgaagatatcatcttcaccataccaggtgtatactcaatggagaataagaactgggattggaatgtctgcaaactcatgactgaataatgtgcagtgtggttgccagagtgagtattgatccaaaccaaacacagaaccatgggcagtggcatagagagaaatcagaatcttagacaatttgggtgtatttcacaactggacattttaaaatgcagattatagttgtgccttagcatttacatagcactctggtcaattgtttgttggctattcctgtgctataccttgattgaagccatttcttgtttatgcttttcttttctttttttcttttctttcattttttatttgagtcagagtttcgctctggttgcccaggctagagtgcaatggcgcaatctcagctcactgcaacctccgcttctcgggttcaagtgattctcctgcctcagcctcctgagtagctgggattacaggcacatgccactatgcctggctaatttttgtatttttagtagagatggagtttcaccatgttggccgggctggtcttgaactcccgacctcaggtgatccacccaccttggcctgccaaagtgctgggcttacaggcgtgagccactgtgcccggctgtttatgcttttcttgtgagtaatgcaggacatatttcagagactctggcaagactatgtcgagaactacaacactcctcatcctcctggtattgagccaggtttaagtagagaaagaaagccctgacatatgttaaaacagaaaaccagaccatatcattcctttactcaaacttctccatatctcctcatctctcccagggtagaagccgaagtctttaaatggtaaccaggccccacacaatcttttctcaacccctgtgaaccccctactatttttcattataaaagttgtcatacataaagagaagttggatgaacagtacaatgaacacccatatatctaccaatttgatctaacgcttcaaaactaacattataaagggcttggagagatggagtcctgggagggttgggaatgaaaagagagaggacctaccagatcagactgttagagatggaatgcaaggtgggctagactgcctattgaactgagttcctcacaaagtgggagtgggcatggtggaggggcgggggaagaggccacactgggctgtggtcagggattattaccagagaagcagttgagtggagccctggcagcccatgtccttgcccttggtcagggccacactcaaaccctccccagcagagcatctattctataaaccctccagagacgcagattacacaacatccttcagtagtctgcgtcaatgtttcaaactgtgaagtgattctccctgaagactaaacatgaggtttcactgtgttctttcagtacatttcctcctgttcttttcttgcaagaaacagctatctgggcaatatctcctatgtactgacgtcaacaaatttcaaggcaaaagtactctgtcattttcatctattttttaaaatgataattattttcttctttaataaccttactagctcttcggaacctttaccacatcccatagaaagaggcaggggttgcaattgaaaacacactgacagattaaaccccagttctcttactggatgacacagagctagttactaaccttgctgaggatttgaaaaataacaacttcctctaaggtggttgtgaacactcagtgaggtaaggtacatttgtgccctgaatgtagtaaatacttgaagttttagttatccccactgtgttgttctagtcagggctaattacctggactgatattttatgttttatttagttatttctgatttctttctttcttttttttttttttttttgagatggagtctcactctgtcacccaggctggagtgcagtggtgcaatctcagctcactgcaacctctgcctcccaggttcaagcgattctcgtgcctcagcctcccaagtagctgggattacaggcatgtgccaccaagcccggctaatttttgtatttttagtagaaacagggtttcaccatattggccaggctggtttcgaactcttgacctcaagtgatctgcccacctcagcctcccaaagtgctgggattacaggtgtgagccaccgtgcctggcctatttctgatttcttctacttgttatggagaccacagcttatacatacgtatacgtgtccaaaaggattagctatttcgtgcttgaatgccttgggagctttgctactaggaataggtgatttactattctccttggccctgactgagatgtggggaggtggaatcatgcacacacgttagttcctgagaacactatgtttgtaccactatgtagatcagagctcctcaatcttggtacttgacatttggggccaaataattcttggttgtgagaggctgtcgtgcatattgtataatgttaagtagtatccctggtctctaccaactagatgcagtagtactctcccctcagtgtgataaccaaacatgtctcctgggagacaaaatcaccctggttgagaacccctgttctatatctctgatatagtgctttgaagagcataaattactgccataccaactgaggttgtcatctgtgagtctgtgatctctaattactctctatcctgaatattctgaccctactgagaaaaacatagcctattaccaagatagaatagagcaccagttgtctcctttcagtgaaaatcaaaaagaaaaaatgcagtataagaaatgcaaggacaaactatatttgtaattcatgtaagtttgatgttatcatggaaccaaagatttttatgcttccttatgctttaactgtccctctccttcatgtctctgataagaattctctcaaaaagccctttctgactacaaagattctgggcctgaggacagattgctaaggttcaaatcccagttccattgatttccagctgtgtgaccttgattctgtgccacggtttcctcatctaaaaaatgggaatgatgctattactcacctcatagggttttgtgaggatttaataagggagcacatgtaaagcagttaaactctgtttggcacataataatcactcaacaaagtttggatattattattgctagaggtagtagtagtctctataattcagagtctcatctccgtcagaaagcctgccggactccatgagcccgctttgacctcttttatctctgaaatcatagaagttcttataattttatagatcaaagcttattcacattatcttttatctgggccatctgaataaaatttcccaatagcacaaggatgtgtgagccataaattacatgttctacatgatttgcattttctcatccacctcataaattgggtaaatagttgttaacaccacttgggggggtctatatacctatacaaaattggatagtggtttggggaactctctcctctcttatcagaatttcctcctcctcttgtcctctcttatctggttatttcatatgtagtcagtaggtagagacatcagcatgatattggtactaacatggaagctcccctgaagactgaggcaaaattcataaagatgatctaggaaggtcaaaaaagctttgcacaataccaactaccaaatccagtgaaaaggctaagcagagaaagacactcactatgagagttcctccaggtatgttctcaaatttccaatttttataattgcttcattacttcatccttgtagaggttgctcagtccttgtttgcctttcaaccaatgttgaaaaacactggtaatcaccaagatctaaaaataacatcttgtagcatgatggcaatatccaaatttcaaggcaacagggccccaattcctttctgcattgaatccaaattaatattattaagacctaccacattttgactgtccccagaaaagatgcaaggcttccctagaatttgcactgtgtaatttctaggcactgaacatattttgcagccaaagaacactcctgatgggattgaagacaagttggtaaatcttaggagggtctttggtggcaaagaatgaagtaggagtaaagctcttacacaacttagcttctagtttttatatattcattgtgacattatcattgtgacattacatctctggcatcatcaaaaaccaagggatttttatttaaaatatgatacacgataagacctcagaaaagttcttataatttcaagtgcatgtattcatgtggcataaaattaaagcccttaccctttgacttttgactttcagattggtcctctttgaagttcagatattaagcacctgaaaatttacaaagtaacttacgcattccctatgtctgtaatttttggaaatgagatggttctatgacctctgatatcttcccccacatctccctcactaaccacttgcttaggttctatatacagggaaaacaaaggagtccttcaatcaaataaacaatagcatccttgggcaggggaccagtcttatagaataatctagagcaaaacttcaaagccccaattaagatcccttctcttactatagaccagaccagcaagcttccctctgagcctcacccgggtccttctacctttccagagatctgatgtctccaaagtagttcccagccttgaagcttacaatttctcatgcaccactggtgttaataagttcttatctgggacattgtgtgtcatagaggggcacaaatggaaagagaattttatactttgtagaataattatcatatattctgccatgccattctgtctttgcaatcattattttttttctctctcaggacttttggcatattatttttgtgatgtactaatataacaatttgttaacaggagtggcatgtctgagaactccaagtcataatcatcttatcctaattctcttgatacattgtttgggaaaaaaatgcatccaccatggctgccaaaacagcttctcggtttagccagtattctggcctctttgcatgtgtctgtgactacttgactatctcatctttgctgaagttaatggctggttattaggaccacacttaataaatagcagctcctcaaggaatggcatggactcttgatagcattctaacctcttgagttcagagccctccagattgtggaataaattagcagtgctagaattaccagccctatgttttgctgacccactttaaagagtgtttacatgtcctgaatcttgatctacaatacttatttttatatgccattttatatttatatcctagaatactattattttatggaatattccactttgatatcccaattagaacaatctcaccatctagggattctgataaccacaactatgggaaaatcagttctagtttatggccacagatgggccagcattgggtaggagtgaagaattaatgtcatattaaaataaaaagtaggtgaataaatttcagaatacatttttataaaataaaaattattagtgaaaccaactggagggatttccattgctgtgacctcattccttgacacccactttcagtcatcagtgagctttggcatcatggtagttgcaacataaatatggttcttttcatacaatgctggagggtcctcatttctcttgttgcctaaaacattaactgtgggctgagatatctggctcagggaaaagactaaccagtgttcaacacttggcttggctacttagtaaataatagtatgaccttgcgagaagaagcagcttgattgaggtaaacttcatttatgctctcaccacccccgctttttttttgagatggagtctcgctctgccacccaggctggagtccagtggcactatcttggctcactgtaacctccacctcctgggtacaagcgattcttctgcctcagcctcctgagtagctggaactacgggcgcacaccaccatgcccggctaatttttgtatgtttagtagagacagggtttcaccacattgtccaggctggtctcaaactcctgacctcaggtgatccaccctcctcagcctcccaaagtgctgggattacaggcctgagccaccgcgcctgggcctttcattttcttcatctatgagaaggtagtaacaataccatttttacagggtcatttggtggattcagtgacaacatatgtaaggcatttagcccaatgcctgacttacagtaatcatgctgtttttctctcacgtcttttaccatagtaacttgaatcatagcttccaaatctcattttgttccagaaaatttttccacctttttttcagttgatactaatctctatccatcacattctgtggatgggcaaataaacagatgaaatatgccatatgaaaatgaattatgctacacacagggtagacaactgagctttccctcttcttcctccttactactgagttgctgaacaatgtctgtattcaataaatatttattgtgtgtacctataatgcaccagccacttttctaagcagctgaagatatagcagtgaacaaaatagagaaaagtttctgccctcttggagcttatattttagtggggagagacagataataaaggaatacattagtaaaacatgtaattatgagtaccgtgtagcagatggtggtgtcatgaaaaaaaataaaggatggaagttgtataaatagtgccactatatgtgtatgtagtggtgatagggaggataaattttaattagttgattgggtagcttgagctttactcttgactcattaaaaaagagaatcataatatttaaaaaaattcaagtcgtcagattgacaggaggcaaaaacagtgtttagtaaaattgggactttataacgttgaccagcattcctgggaccaatgctggacttagtaaaactggctctattgagttattgttctgttagttctctgtgaggtctcttcttccaacttgatagaagggactctacgagtgaataggaaggttgaggttggagtctgtaactaaccttgaccaagctccactgatggctcagaccagacaaatcccttagaagacgaggagggaggaatacaatgaggtgtccaagatgagcctgctagacctcaagaagacaactactgttgaacatttccagttggatcagaaaacatgactcatacacaatccatagactgaggtggaggccaataaaataaaggggttttctaagatatttagagtcaattaggaagcattgaccaaagtctaaaattcttcggtcaccctcagactctagctgagatgccagagctggttccacaataagtcttctatgccttaaatgcccaccagtgttgcagccttgacctggattgcgtctaagcccttgactttgactatctgtcatgtcagctgactttcctgaactggcaccttgcctcaacttaaccaccctgttcttctgagatgtttctactttgccataaaccagtgaggactacaatgctctggccaagatcctgaaactcccgtttctccttcctgttcattccacagatggcttttttttttttttttttttttttgtcctgggatacatgtgcagaacgtgcaggtttgttacataggtgtacatattccatggtggtttgctgcacctatcaacccattatctagtttttaaggcctgcatgcattaggtatttgtcttaatgctctccctccccttaccccccacctcccgacagaccccagtgtgtgatgctcccctccctgtgtccatgtgttctcattgttcaactcccacttatgagtgagaacatgtggtgtttgtttttttgttcctgtgttagttttctgagaatgatggcttccagcttcatccatgtccctgcaaaggacatgaactcattcttttttatagctgcatagtattccatggtgtatatgtgccacattttctttatctattctatcattgttgggaatttgggttggttccaagtctttgctattgtgaatagtgctgcaataaacatacgtgtgcatgtgtctttatagtagaatgatttataatcctctgggtatatacccagtaatgggattgctgggtcaaatggtatttctggttctagatccttgaggaatcgccacactgtcttccacaatggttgaactaatttacactcccaccagcgtgtaaaagcgttcctatttctccacagcttagccagcatctattgtttcctgactttttaataactgccattctatctggtgtgaaatggtatcgtattgtggttttgatttccatttctctaatgaccagtgataatgagcgttttttcatgtttgttggccacataaatgtcttctttcgagaattgtctgttcatatccttcacccactttttgatggggttgtttgtttttttcttgtaaatttaagttccttgtagattctggatattagacctttgtcagatgaatagattgcaaaaattttctcccattctgtagatggcctgttcactctgatgatggtttcttttgctgtgcagaagctctttagtttaattagttcccatttgttgagctgggcatggtggctcacgcttgtaatcccagcactttggtaggctgaggtgggtggatcacctgagattaggagtttgagaccagcctggccaacatagtgaaaccccgtctctactaaaaacatacaaaaagttagctgggcatggtggtgggcgcctgtaatcccagctactagggaggctgaggcagaagaatcacttgaacctgggaggcggaggttgcagtgagtcgagatcgagtcattggactccagcctgggcaacaagagtgaaactccatctcaaaaaaaaaaaaaaaaaaaaaaagatcccatttgttaattttggcttttgttgcaattgcttttggtgttttagtcatgaaatattttgccatgcctaagtcctgaattgtattgcctagtttttcttctagggtttttatggttttgggttttgcatttaagtctttaatccatcatgagttaatttttgtataaggtgtaaggaaggggtcggctttctgttttctgcatatggctagtcagttttcccagcaccatttattaaatagggaatcctttccccattgcttgtttttgtcaggtttgtcaaagatcagatggttgtagatgtgtggttttatttctgaggtctctgttcacagatgtctcttttaaggagagttgtttcaagtaactttcccccactatttatccttccaatttccctcctgaggtctagactttaatacttttcagtacactgggattttgaagaggtaagatattttctttttagttttagctgagagacaatatttgagtaaccatctctagctgacaattctcaaatttgtatctccatccttgacctctctgccaaactccagatttgtatatccaattgccttctcaacatctacacttgggttgctaatagatatcataaacttaactaaactttcagtcttccctttaagcctattttaccagcaatctttctcatctaaattgatgttaatttcatccttctagctgtttaatccaaaaacccggagtgatctttgactctttctttcatagcctacatccaattcaccagcaaagcttgtttgctttacttttaaaatgtattaaaaatccaatcacgtctcatcaactctactgttaccaccaagccaccattatatttcatctggattattgcaacagcttcctcagtggtctctctgactgtagtcttgccccatatgtggtttgttcttcatgtagtggccagagggattcctttttttttttttctttttttttttttgaggcagggtctcactcctgtcacccaggctgcagtgccatggtgcgatcacggctcactgcagcctcaacttcctggtttcacatgatgctaccacctcagcctcccaagtagctggggctacaggcacacaccaccacacctggctaattttttgtagttttagtagagacagtgtttcaccatgttgcccaggctggtcttgaactcttgggttcaagagatcctcacgccgtggcctcccaaagtgttgggattacaggtgtgagccactgtgcccggcccaggatttcttttaaatgtaagtgatatcgtgtaactcttctgttcaaattctaccatgcatcccatctcacttaggataaatgctcatgttttgcttctaaagtcttagatgtaaatttatactagtgagttccttgctctagtgctatctgatattttgtacagtataggtatcaaaataaattacggttactgtggcaggcactgtactatcataaattcaggccagtaacaatcagtataaccttcttggaatccaatgtttattctcacaaatttgaactctgtctattgaaactgtttatcagtttggttaatgaagattatctttgctgtatctttagtgtttgaaatgtttgttttctggtgccataaataaatagtatttgaacatagatttattttttttagtaaggctatttttatactttctgtagaaagggtccacttgccagtagttttgttacgagagtataccgaacaaaggagatagggttatttataacttgacgcgtctaccctactgttgtgtctggttcttattggctggaacgggacctcacattttgtgtttgtcttgactggctagcaacttagaactttttaaaagaggcaaaggcagaggagaacaaaggaaggaggaagtaacttgtggaatgttgagaaaggtaaaaaccccttcaaataaggaagaggaacaggctatgacctaatgcttgtttggagcagtattaaggatgccagggcaaatatttaggttaaattgtgggagctaagaacataaagtacattgatttttttttattatggctagcagatatttaagaatgttagcacaggtttttgaatgaattttgcttctaagagaagctattatttattcctaattagacagggaggaaagtctttgaagaggaacctctactttactttttacatttaggaaaaaagggactgctatgcaactaaaaatgtaaacaggaaagagctttagcttttcctttgggctgaaagcccaactagttatttattttctataaagcacttataaattcactttgtgtgataaacatgtggtcaaaatcaattgtactgaatgcaaatagttcttgaccatgcaaagttttgaatttctctatgcattgtgttcctgatacccttccttgttctgtcagcagtattaatcaataagaaattgaacaaaacatcctttcttttctgatgtttaccaaagttatccacccaacttggggaactggaagaggtttctcctatagctatttggcaacctcagcatttgtgactcagttggaaggatataaaaagttgaacagtaagggaaatatggatttcaaagtctatgtactttgctctagcagaataaagacccaccctcggatttcagcctatgaaaagggaggtttctagttgaactccatggttatctagtttccatttaggttttagcagatagcagatcagaaacagggaggggacttgtggagagcagggctactgaaagttgatactgtgactgtgaactcagaatgtgcagaaacagcagaattaatagcaataaagctgttgagtcatttaatgtaaagccttgaagacattgtcacattatagcttagccagtaaatattatttaagatgatgacactgagtcatctgaaatataaccctaaactaatgtccaggcagcatcgcgatcgggatggaatatattgaagaaaaatttctaactaaaacggtttaaaaatgtataactggtgtaatcaaagcagtacattttaattttgcttatggggacctgcttattctcagaatatgacagattgatgaggacttattagacatgcatgaagttagcagataactgaacccatgactctgtggagagaaaaggtctcttaagcaatctgggatcaaaatgtagccccattgcctatcagttgtggaagtttgaattaatcatgggaacagttggtttccttaattataaaatggatctcactatttctacctgtaaagttgttgtgaagagtttatatacaggagcaggtacaaagcatatgcaaccaacaaatacaacatctgtaaatattgagagggagtcatttacctttatgactttccccttcttcctcaagatagcgtgacagactcaagttttctcttctctgtatgtaggaagttttataccaggacctgaccatgactgggtgatgaaagcacgctgagaggttctctatggctgggctcatgtagactactggctgactcccctttcctttcatatatgactctactcattccattggtgagctcctccatcctgctgtttcagcacaaactagcagacttctcctacaatgcaggatcctatagtccaaacagacctttgagtcaaactcccagccagtctctgtctgctgcagtgtctctgaaagatgaccttgcagatggtttgattaaacattccagtgtggaagttctctaattattacacagctcctttcattttctgtggcttttattactaggcttacaacattactctctgtgttcttatgtattaattcaagtaatatgacatccctatgaaatggatataaaactatcattggccttgaaataaattaggtgcagggaggatattacctgcctaaggtcacacagctaacaagtggcagagtaaatccactatgctaagtcttcttctaaatggcagtaccatgatggaagaaagctgtccaccctgtcttctcttccatatctaaaccattctccaaattatattattttagatccatctgctctcctatttattttcctctggatgctctcttgtgcgtctctaaaattgtggttcacagaaatgaatgttatagtctagattattttgtatctaatgcttatattagcaaagtctaatatagtataagatgtaattataggcccagtaatttttactgggcctcctagttggatcttgaagctcatactaagcttttattcaaccagcttgactgggctgctgcagtagctcattaactagcctccctgcttccatccttacccctttagtgtccattctctaagcagcagccaaagtgaccctgctaaagcttctactagatcacttctctgcttgaagccttccagtgtcttcccatgttgctgagagtaaagctgaagtctcacagttaattgtctttaagacctccctggtgtgaccctagcttcttccttgacttcacctcctgccactttcccctgcacactgagctccagtcacaaagacctctgatgttcttgaaatctgcctacatcctttacacttgctgctccctctccctaggaaaacttcttcacccagagatccgcacaactttcatcacttctttccggtctttgctcaaacgtcaccttctcagagaggccctccttatagaaaatccctctccccaccccataattctctatcctccatttcttgcttattataagtcatgacatttatcactatttttgtcttccttctattgtcacctcatataatacaatatttattagcttacattcaaaatctagaatagtgtttggcatatagtaggtattcaataaacaatgattgaagcaagattcttgttgttttggacatgaattgttgtttaccagaggactgcctaatcctgtactgtgtaattgtttcaggggacatttgggtctagtcatagcattttacattctgttaaatcccttattgaaaaggaagatacactacgtctttggatatatttttttactattgtattctatcagagaggagatagggcaggcagcatctgacttattcctagtgaattcctaatggaccagagatatatttttccaagtgcaaatcacattctaaaaattttactttaggccgggtgcggtggctcatgcctgtaatcccagcactttgggaggccgaggcgggcagatcacgaggtcagaagatcgagaccatcctggctaacacagtgaaaccccgtctctattaaaaatacaaaaatattagctgcgcatggtggcgggcgcctgtagtcccagctactcgggaggccgaggcaggagaatggcgtgaacctgggaggcggagcttgcagtgagcggagatcgcgccgctgcactccagcctgggtgacaaagcgagactccgtctcaaaaaaaaaaaaatttactttatagctttactaggtgttaaggtcaaacttccctttaccattatgaaatccaccccctcctccaatctttttgaaacatagaacatctttcctgcatctttggtcttctaattttttctgattctccaatttttctcaaagacaatacattcctttagaccaattgtttttctgggtctcatctcctgagtcttcagtgaattctttccattctcggaatgcgctcagagagaacgcttctggggaactatcagaacattgatatcttctcaaaaaagtgtgaacaaaaattccaataattcagtcagtgcttgcagttggccattctgggctagtttttctacagaatcactgtagactattctcccttttcccttgcttctcaaagtttcttggcttctagcattttcctgcctgcctcactgctgtcccaggctccttgttcatttattcttagctttcagattttatatcttgctttctccatttcagtttgatcaagttaatcgtattttctgcctcagcgaattgaatttggactactcccattttttccactaaacccacgtgtttcatttaggaacttacctgtgtagaaatttatttccgcttttattacagtaacatttcaaccttttcttcagatactcgtgtgctgtcctctttcttgcctgttttttctcctattgtgtgcaaggagtgagtggatctaggtttcctggaatgtgggaggagcttttctgaaatttctgcactgattttttttttttaccctcatctcactttcatttaaaattctttattattattacttatatttgtgagctgctttatttttttaaaaggagagtatattttgattcacatcaagggaagtaagagtccacctctttttgatgcagtgtaactaaatctgtcatcttcactgaggcacaaggccctctgtcacattttatctatgcctccactattgcttttagactgttgactacaagttagttaagagcctatactctagaatcagaatacatagatccatgttctgattccatcatttgttagctgagtgagatatagcaatttacttaaacttagtttcataattcataaaatggtgataaaatatttgcctataggtttcttgtgatgctcaaatgagatattataacacaaagcactttgaatagttacttataattgtttttttcctattctacaataatatacacaactttggaataaggaaaagcagtggacatttttttaaaggcttgattattgcaatgtcacctgaacctggaagacttgtttttctgggtgaagaaatattttctctgtgtcagagtttcacaatcatcgtcaggtaggcccttgaaccctgccatttaaagtgctcctctctttgatctgtggcactcatacatacactctggcaatgaggacactgagctcgcttctgaaatttgacaagataaccactaaaatctctttgaattctatgttgttgtgatcccatggctacagaggatcaggagttgacatagatactctttggatttcataccatgtggaggctttcttacttccacgtgaccttgactgagttttgaataggtaagtgaaggagaaggaggcactcaaagaagtcagccacagaaccagtgtgagaaataggaaatgagcttttttaagttttgcaaaggcagatcaggagagttgacctgtggagtggagagtagtcataattttaaaaaatggccatggaaattaaaactgatcagaaatggctgggcacagtggctcacgcctgtaatcctagcactttgggaggccgagttgggcagatcacaaggtcaggagatagagaccgtcctgactaacacggtgaaaccccgtctctattaaaaatacaaaaacttagctgggcgtggtggtgggcacctgtagtcccagctactcgggaggctgaggcaggagaatggcgtgaacctgggaggcagagcttgcagtgagccgagatcgcgccactgtgctccagcctgggcgacagaccgagactcagtctcaaaaaaaaaaaaaaaaaaaaaactgatcagaaatatgaattccataaagacaagggcaaagtcatgtatttggaagggaaaatgttgcccaagtagagagtttctgggaatatgatcttgaaataaaaataaatgtgagataacctattaatgaaattgtctgaaaaccatacaaacaccaacattatcttcatgatccctagttctagacctctttggtcactgtaaaattataacattttccgtgtatctttaacagctttctaggaaaatattaaccaaaagtaccggttttgatttggccataaagtgacaggagaggtaaggttctctaggattaaagaataacgtattcttatttgattcactttaaaaaattattctaaaatctgttacatatctgtcctctccaggatgaactttatattggctcagcagattgtttactgtgttcttcttctttttctttttttggtctttcctgctcagcgcccaacccaagttcaaaggctgataagagagaaaatctcatgaggaggttttagtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatggtgagttctcatggctctattgggctatttgttgcctcttaaagattagattactgcccgtgaaactgaaaagggaaatcaaaaaaaatgctctgagttgtgagattggatgtttgcctccatatccctgattttggagtcccagataactaaacttaattgaccctggggttcattcagaaaattgttacaaaataattcactggtctcaatccagatgcttggaaacaagagaatattctttataaagttaccaccataattctcatcacagtcaggatttcaaagcatttcatggaaatgccataagttattgagttaataatttcctttagcctacaatatcaataggaatatctaaagattctctacaaatgatatattaactgaaaaatgcaactggaggcttagtgaaagaactagtaattaagctaatcagggcttgggtgaggctggacttgggaattcctttcctctttgtcacagacctccaagggactccaaaaggtcacagaatccaggcagagccctccaaaacaaaaacaaaaacaaacaacaaaaaaactatgattaaaccagttctgacaaatatcagaatgctctgggaaagccagatgctttaacaagtgcaaggatttaggaatttcttctctcacagatcccaaaacagtatccttaagtggtgatccttatgtcaagctgtgagaatatacctcaaaataatagaaggcatccaaacttgatcagtatagtgtcataaagctgctgatgtagaagtgtggagaagtccatcagatagagatatggaaaaaaaagcttggttaaaacaggtttccaaagttctctccctttctctgccattgtggtatgtgtggttgaccatttctcatcatgttttcttacaagactttcaggatcaagcaattcctcgccaaaaaacaaaatcaaggacacgttgtagctatttcttggtgagttaccagaagctgtgaagagagagcatgaggcccatacttttttttttttttgaagggtctcgctctgttgcccaggctggaatggagtggcatgatcatgattcattgcagcttgacctcctgggctcaaatgatcctctcgcatcatttaacatttttttttgtagagatgagatctcactttgtctcccaggctggtcttgaactcctgggctcaagtgatcctcttgcctcaggctcccaaagtgctgggatgacaggcatgagccactgcacctagcctaccacacttactctttgcgtggtctctagagcttcttcagcccccctgctgccagggtccctagggtcttgggcatgccccatttctgaacctattcctgtctccagggatagagtcacagtgaactcagaccagggtaattctaaactgctattctgtatatacccaaggaaatttcatccctgtctctaatgagtctgtcccatgccccttaatacacatgaacacctacacacaggtacaagtcggggaagatcactgtgcagcacataaaataaaattcaggggcgagcactttttttcccatgagataaagttaggaaaaaataaaaatgttcatacagattaaaaattttaaatgttaaaatattaaacccccaatctaaaataaagaaaaaataatttcttaaatcctaagcttgagttatggaattcacattcttataatttacattacctattttgttaaatggaagacttttttttttaatatgcaagtacctgggcatatttagaaagtaaccaaggcagcttttgtctttgaacctgatgcggttttgacagctctatctggaaattgagccagcatatgggagccaagggtgaaagttttcctgggagatttgggctccaaggtactggatagatgctaaagattccattcagtttttcattttctgtcttcatggaactctcacacctacaggggaagaaatggtttagaggttactaaaagtatatagcctggtttaggtgatagtgagctatagaaaaagagaaaataagttgtcaatactttaaatctcaaacaacttcttacaaaaggattttaatagtttaaatacaattatcacaagttgggtaaagacaaccagacctatggcctgataaatcatgtatttaatttatgcatatagttagggccaaaaagagcagagtatctttttgaatgttttaaaatataatctaggattttatgcctacagtccttgaaattacaaaggatctctctcaagttgggaatatgctatccattgcagttacggatgggatttagagactgttttaatttcttgtgattatctcaaacaacgtgttccttactcccaaaactcccttgtatggctacagaaactgttgcagttttagttgcttctcccctgcctttgcccagaggtagtgtggaatgccaccaattttatctgtaatggttcctttcaagcctggggctatgtaagctgctatatatgcttcattccattcatcattcagtctaaatatagctatcaaccactaaattatctgataggtatgcatgaactgactgattctttgcataactactaagctatctgttttatttaggcttgggagccctagggtaaggatacggaataaattgtaaatgatattaaaagaatagcttggcaaggaataatacctcctcaggggccaaaatgcaaggcaacacaaagtgatttttttggtgatataattataacacatgtagtatggtagtgataactaaaataaaattggatagcctcaaaattcctacatgtagtatgataaacaatgttctaagactgaggcctttgggaggtagtgagttatttcctatagatagagagtaaaattattcctataatttggtatgaaagttttaccagaaattttgcttcacagtaaatcatctggattgggtgaagccaggtagggattgagaagtgagtcaggtgggttgcatagatgagtttgtgctgcagaccaaactcctcttcccatagctcacttcttcccactcctgcatgttagagagaagactagaatgtgtcaaatacaacctataggataatagttcagagcattagcttcggaagtaaacactaggttgatgccctggttcttctaatttctagctgtgtgacctagggaaagttacctgctgtgtgcctcatttcccttatttgtaaagttgagacaataacaactccctcacaaggttgttgtgaagattaatgaaaaagcagtttctgagaggttgtgagtgttcagtaaagaaagaaattgctttcctttttttttttttttgagacagattctcattctgtcattcaggctggagtgcagtggcgtgatcatggctcactgcgcctcgacttccccaagctcaggcaatcttcccacctcagcctcctgagtagctgggactacaggtgcgagccaccacacctggctagtttttgtattatttgcagagatggggtctcgccttgttgcccaggcttgtctcaaacttctgggctcaagcgatcttcccacctcaacctcccaaagtgctgggattacaggcatgaaccactacacacagcctattttccattttcactgccattattttctctgtctcttgtgtggctcattactgccacttctgtgcaccatttccatcatgtcacatgcgtagtcaaaaccctcagtgactgcctaattctcagagggtaaaagtgaaattccttggcctggaaagtgcaatcatgcctgaagttctacctgtgggagctgtactagttatctattgtgtaaaaagttaccacaagcttagcagattaaaacaacacatatttaatatcccacagtatctgtgggctaggagtccaggcacagttcagctggatcttctgcttctaagtctcatatggttgcattcaaggtgctggctagagctatggtctcttctgaggcttgactggggatggatatacttccaagccaatgcaattactcagaggccgtctttaattatttgccatgtggccttttccatagggtagctcacaacgtggcatcttgctgcaacaagtcagcaaaggagagagtcttctatcaagattaaagttaaaatcttatgtaacgtaaacatgacatcctgtcacctgtgttgtatgctattggttgaaagtaaatcacgggtctgctcatacttatggggaggggatcacacaagggtgtgaccaccaaaggcagagatcatgggagcattcttagagtctgtctgccacagggccaggatcaaatgctggtggctgattgccagctccactgtttatttctctcagccgctttctgtatgtacaaaaaaggagagagagtgattttctacctcatagtgttgttaagaaaatcaaatgaggtaaattattttgcaaagtattctctactcatctgactcctacctactttcaggtctgtcttaagttgcaacttctgcatgagggcttggccaagtatggtcacctgcatgaatttcctctttcttgtaacagaaactgcttggcccactcataatttatacattattatatactcttatgtttcattaatgaaacatttataatgcatatcttatctgtctgataaaatggaaagcaatctaaggacaacgacttttcccctatacataacaaaatataaaataaatcataacataataaataataaataaataataaaataactaacatttgttgaatgattaggatgtgttaggcactcttgtaagcatttcatttttttgttacttcttcaaatccttacaacagtccttggaggtaagtcctattattactttcattttgcagataagacaactgtggctcagagaggctaaggtacttgagtccaggagcagaattcttactttcctcaaacgttatctcttttgtgtggtctagggcagtacaatataccaagtagttgctcattttggtctttggtaaatggaaatgaatttgtaagttgagaaaagactacactacagtagtttggagaagtccactttccaactgatgttcaagattcaggagggggggaaatgtgtcctggcatcatcacttgcttgatgatttcttttgctggtagaaaatcaccatggctaccaatggccattttctgggaagccttttttgggaatcctaacttaagatcaaaatccctgggctaagtcacggcagctaggaggttactgtgaaccggttctcatttcttatggtttccattgttattagggggctgtattctcattaagtccactctgagctctactggattctctttttagagcactcgtctttgaattcttggccaactcatctatgtcacagcactaaggctataaattccagggccccacaatgtggattatctgtgagaattcatttccagttctggggacaagacccttgggaaaaccaggcaataggccagagagagacagagagagagagagtggtcaaaagtggcctggtcactcttaacctaaaccttgaccttcagcggagtagaggactaaatcactcaggaaatattaaatgtttcaccagatattatttggactatatcttctgttctatctcttcaagcaatgccattccaacttcaattttcttttctgtcatttcagaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacgggtacgtttgtgaacattttagcattgatcccaagagttgaaatatttgggaaatattaaacattaatatcacccacagcagctgcctttatttgaaaaaatagtaatgctgatgttagaaaaatctctctctatgcctaggtaaggtgtccatataatttattattcaaactggaatctttgagagtgaaagacactggtaattattatattcaaacacagagttaaactgagattgtccctggcactgtggtgtgtatggacaccccctagtgttaggtcaccttcctgaaggatcaggcagcctgaagaatacagttagtttatgtcagtggttctcaactgggggtgatttagccctccggggatatttgacaatgtctggagacgtttttggttgccacaaatggggatactgttgacatctagtgggtagggatgctgcagaacatttcacaatgcatgggatggcccccaacaacaaagaatgatcttgtccacaatgtcaacagcaaacttttttttctgtaaaggaacagatgacagtaaatattttaggttttgtgtatttaggtattgtctgtgttgcaactagtcaactgtgctgttgtatcaggaaatcagctgtagctaatatataaatgaacaagtatggctgtgttcaaataaaactttacaaaaacaggcagcagacaggatttggtctgcagaacacagtttgcagacctctgatatagcctattgaaatatttttaacctaaaatatttagctatttaaaaagaaagtcttctttctcagttgcttgtcctctggaactttgtatgttctaatctgtgaatataaaaataaaaatgcatcaaaaaggcaaagataaaaccatgtgaaatgtatctttctacaagttaacatttatttttctgcattctttgggtccaattatccaactaattatcaatgcactttactttatccattctatgtttctccattttttcccccaacaaggacactataagaaaccttgcccattcgcatatagacaatctggtctagcctaggtggaacctagaaattctattttttaaaaaatccccagataattctaatgtgtagccaggactgaaaaccactgaaatgcatgtaagagcttaaagtgtttgggaagaggagggactcaaggttgcttagatttgatggagaggaagccgtggagattgaagaagagctggagagagaccatgaggggttttgtgccatggtaaggaatttaggtttgttcctttggactatgggatcgtgtggaagttttaaacataggatctatttcatgttttaggatgactctcaaggcagaaccaaagggaaataggagaaggagaaacagatcctttaatccagtgcttcttaaatcaatctgagggagagaaccatttccttccttccttccttcctttcttctcttcttccttccctccttctctcctccctcccgtctttcctttctccctcctttattcctttatttccaatacattgcagatcaatacttgcttaagttcattgattacatgcttagatgtcatggaaatatcaaattgttctccaaagtttataaacttttattctcttaattcctgtacttatcacagaatggaaacaaactgtcacttgcactggtccacggattacacctggagtagcactgcttcactatatcaccttcactttatctgcagggtaaagtttaaaaactttggtatggcatagacggtccctcgtgatctggcccctgcttgtctctccattctcatgtctttcatttcccctaatgaacaccattgctccagtttcattcaacatctcactgtcccctgagaatgccttgtcttttccaacctccttccatcttttagactcttccctctacctcccttctctacatgaccagtgcctacttcttctccaagacacagctcttatcttcccttctctttggacccattcttgaatctcctggttattagttattaatttctccctgctcctatactaccgcatcactttttggtgacagttatagcacttgtcattctgttatgtattaatagttattgatttcatggctgcctcactgtcctatgactttatggtctttgacttgttcacttcagaattgtcagagtgtaggacatattccaggttcaataaatgctgaatgaaacaaaaaatgcacctctctgtgtatgtgtggataagaaaacttctttgggttttggtagatctggttatttcaattactccatatatatttattatgtgcttatagtactttgtgctagtcgacagtggggaaacttaactgggctattccttctccacatacagaaagatacatggcaaagaagtaaattgctgtattttgagtaatcaaaaaatcatgtggtcaaaaggatatctttatattagcattctcttcagcaaaatttccattgttaacattgtttattatctttaatttgcagttgaacacaattctaaatacaatgagcaccatctacagtactggaaaagtttgtaacccagataatccacaagaatgcttattacttgaaccaggtaggctactaatttttagtagtgattatgaaatttacttttctctcagattttaaaaatgattgcagatatgtgtgtttcaacacatagatgctcttttaacttttagtaaaactttcttgtaaattgtatcacatttacttaatttgatcaagtcagtaagtaagtcatttcagtggtttattttctctgcagacatattttggaactataagggcaaggacatgaatgaatcatgagattttatttaaccactttatgacctgatgtctgccaaccttacttttaattctttaaagttcatgtagttctttacaaataaaatttttacaaaggcatcttttcttgtctttgtgtgctttgggataacaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcgtaagtttgctgatctgtagaggtcctgaagctctctgtgtggccacggggctatttcttctgcattaccaagcctgccctttgatatcagggatacccctcaattaacaatcatgtcctcaacaattcagattacctgttttcaaaagaaattactggggaaaaagaaaaggtgcactggcattctacaattcaagaagctcaagaatgagactcaactacaaggcctaaagtaaattatccaggaattatgaattataagtaatctaggaagatctcaggcccctaaaagttcccgagggagtctggggattaagggggggaatcccatttctcaggatgaaagacagagtcactgccttttgtctaaagcccccacagttcactttcaaaggtcatttgtgtgtctaccttagggtgataccccagaaagaaaaaatatcagattagtttaggctggaaatgtattattttctgcagttgtaattatatctcagattatgtggtcataaaaaatcttaacataggaaggaatttccatatctcttaacttattctcttccctgaatagtgagctgaagtcatggaatcatgattcacattttgctttgatgatttactgcccactgaggttgagtaaggaagaatttttttcccgcatttgttgttttaacatttctttcactgccacttaaattcagtattcttaagccttaggcaagctggagacaatagcttatcaccatttttaaaacaatatcctgcatgtcctttcatccaaatatttatttctctcctcatcttcctgatcattaattgccaatgtgaatgtaaaaataaaaattattaaattatgtttacaaactttattagctaatatcctgacacaagcttgtgttttctgatggtgagatactaaaggaagcacatagaaacatgctattgatacatataatttgcaaacaaacacaggttcactgtggcaaagtctaagatgagttgggtctgatctaaaaataaaaggccatgaacaaaagaagaatttaaaaacttagagatccagtcacatattttaaagctaaagatacaaactgatgtatcaagagagtacacacccagggttaagagactaggaatgtgcttctttcgcttaccagctgtgtgatcttggacaagtgatttaaccactctaagcacaggttgttggtgggaatataaactggtgaagctgttatggaaaaatagtatgaggattcctaaataaattaaaaatagaattaccgtatgatccagcaatccctcttctgggtatatacctaaattagatgaaattaccacttcataaagatatctacactgccatgttcactgcagcattattcgcaatagccaagatagggaaacaacctaagtgtttgtcaagggatgaatgaataaagaaaccgtggtgggtatatggaatgggatattattcagccttaaaaaagaggagatcctgtcatttgcctcaacatggatagacctggaggacattatgctaagtgaaataatccagacataaaaagaaaaatatggcatcatctcactcaaaagtggattctacaaaaaaatcaaatatacagagatagagtaatagtgattaccaggggtgaggtggagcggggtgggatatggggagaagtgggttaaaggatacaaagtagcagatatgcaggatgaacaagtctaaagatctaatgtacaacataaggactgtagttagtaataatgttttgtattcaggatttttgcaaaatgagtagattttagctgctcttccatgggggggataatgcttaactatgtgagatgatggatatgttaatttgttttactatagtaaccattttattatataagtatctcataacatcatgttgtacaccttaaatatacacaataaaatttatttaaaaataaacataggctgttactctgcacagtgggagttataataatgacaatcttgtatggttcttgtgcttattaaataaaataatacatttaaactcctggcacagagcaagtgcttaaacagagattacttaatgttattattcatttgcatccctttgagttgatgtttatcacaataaaaatgtgataagcctttaaatttcttttaacctcccagcaaggctaatctatgtattttgtgctttctctctttctttcccttatgttcttccctttctctttaaaaaaaaaaacaaacagattatgaggactatggggattattggagaggagactatgaagtaaatggggtagatggctatgactacagccgcggccagttgattgaagatgtggaacatacctttgaagaggtaagcaaggaactgtacacacaaacagttgtggggcagtgtatgggaaagagggacagtgactaaaggcattctggctcattcttaacacattggtcagccattcccaggcctcataaagttatggaatattagagtttgaaagattttagagaataataggacagcccctacagatgatcagaattgcgctgctttcctccttctttcagtcactgaccattaattaccaatagaaagactataagaatgaaatattataagaacaaatggtcctttagattataagtaggaatgatcttcttgagctacctggggctggcactgtcttcctgtgttagcagagctatggactttgctttcttcctcaggacaattgatactattactttgagtaagctagtcttcatagggaagagagagttttgtgaacagaaatgtcccatggatgtttttccttcattttggccccatgcctcccctttacgtgaaccttcaaacttctgatgtgagttaccttgtcacagccatagtttggcagagctatcaaaaatggaagtaagacaaacaactccccaaaaaggaaatgggaagaagacttgaataagtacatcatcaaagaaattacacaaatggcaaataggcaaataaaaagttattagggaaatgcaaattaaagctatgatgaaaatccactccacacccaccaggatagctatgattaaaaagatggacaataccaagtattggggagaatgtggagacactggagccctcttacagtgcaggaagaaatgtaaaatggggcagtcactttggaaaacagtttggcgatttcttaaaaagttaaacacacacttaccatgactcttaggtttctatatgaaataaatgaaaacatgtgtccttatccatagaaagacatacacaaatgttcatagcagcaatattcataatagccccaaactggaaaacccagctgtccttcaactagtgaatgaataaacagactctgatatactcaaagtagccggagataggagtggaactgactacaagaaaccttttgtggttatagaaatattaaaaaactagatagtggtgatggtttcccggtagttttaatttattgaaactcatggaactacacacttgaaaatgggtggcttttatagtatgtaaattatacctcaatgtaaaaacaaaggaagtaggtaacaatcttaaaaatgaatatatattttttgcatctgcttttattttgggacctgtgttctcccaagtatgtttattggtttatgcagtggagttatcatcaagttctaggaagaaggaatacttttaaattttccagaaatttgagagaagaagaattataatttggggaaattctacttgtttaaagaatctcctatttctagtgttgtggaatggaaattagaattggttactctttgtcttaaactttcttcctgggcttttcagattaaaccattatatgaacatcttcatgcctatgtgagggcaaagttgatgaatgcctatccttcctatatcagtccaattggatgcctccctgctcatttgcttggtaagaagccccatgaattcttcgtgtacttggctctttgttatttctaacaataaaattgcctttttgagtgagccttaacataaaacttttgaaaaattctgtaagtggttagtaacattgtagaaatcgcattttgaaagcattttcttcaaccaaaagtctattttaaacaattctctattgtattactttttttgtacataaagagttattattttggctgcaattcaaatgataaaggggttatttaagataactagagaaagaattacaggctcaaatgagagtggaaatggggaaaataatttttagtattcaagttgattggctaagctcttaaggtggaatttgagattttagtatcatattgtcagagcttggttggctgggggtggtcaggggtaacagaaggttatagcttgagcgaaagcttcctaagttcattctagacttcaccaattactcttgctgaaaagtgcttggcttggaatcgtataagtgaagaatgaatgaaagtgaccatcttcccctctcccaccgcagcagctgaggccaaataaaatataaatatttgaaaatttcatgccatgcatatatagcatttctactgtagtcaaactaagttgctggaagctaatctcctccacagccatacctcgtgtctgtcagcaggagagaatgcggagaaaggacctgtggaggcttttaactctaaagtaatatggaaatccttcctacaatgtcaatggcaagcattatttagtaattcagtttctgcttaaacctgtctggtgtgatgggataatttcttcattccaatatagactaatttactgctacatggaaccaatgtaatacaattctaccttatgtggaacttaagcaacacccctctgatgccttccattcattagccctatttccatgctatgaggttacccagaatgtcaattcctattctacaagatgaccttccagatattcataaagaaccatcatataagatacagcatagtacctggtgcaatacattttagtgactattattgttattgtcatgtttaccctattttcttttgtcaagttaaatatcttatacattatcaaccattccttaaaacctcacttttctataattgttctcctttcaatgagctgtactttgtcaatgtccctcctcaattatcatgcttagagatgaacagttactccagatgaggtttgaccaacataaagaaagacatggtaccatgactacctttgttctagacactatacttctattgatttagctcaagttcacatttatgctttagaagccaggtcatactgtttgttggctcatttttggtcttttacatgtggactgtgctttagcctgatgtcacatatatgtttttatgatggataggtttttctttaaaatttaagcataggacttcacatgtatcactactaaattttatgttttaaaatataagtttagggagtacttttatttttttcagagtctcgctctgtcgcctaggctggagtgcagtggctgatctcagctcaccgcaacctctgcctcctgggttcaagcgattctcctgcttcagcctcctgagtagctgggattacaggctcaagccgccatgcccagctaatttttgtatttttagtagagacagggcttcaccatgttggccaggctggtctcgaactcctgatctcaagtgatctgcctgcttcagctctgcaaagtgctgggattacaggagtactcttcatgaagtttgttgccccatttttttggcaccaggattctggtcctgctcttggattgactttttctagatacatcttcttttttatggtccctggccttggaatacaacttaacatttgtttgagatgtttacaaagtcaccaagttaagtacacgaatataggaataaaagtggaatttagttaccagttagtagaagttcctgaatgatgatcttatatgatctcttttgaagccaacactaggaattactaacagcttaatttgtaatattttgtaccatggatggtagattatagaaatctctttctaatttacagtgtcaatgatggtccatatatttaataaattatgtctacatttctgctgtgttgtcatatactaacagattcttttttaaatataggtgatatgtggggtagattttggacaaatctgtactctttgacagttccctttggacagaaaccaaacatagatgttactgatgcaatggtggaccaggtaggaaaaagagcccttaaaaactaaatctaaattctcaacttcatttatttttatgtcatccttctatttttatttttatgtgaaaccatgtaaataaaaaatttaagctaattgaaatttatttcttatgtagctatagtttcatggaaaagcagtttacaatggcctcagataacttgaattgcaggtgtagtattgatctcattgaaaggtatttgaaatggagattacctgagttttccattaagcggtaagatgtagaattgtcaactggttggtatcactcattccaaagtttgtcattgctaagtaaccagccagtaatctctatggatagtaaaaacaagaatattatgaggggatttttgcatatttaaaaaattattagtgttaaaaatattacaagaatctttaaaacaattttaagtaacatctttattactactttggtttaattatttaaatgtgctaggttcctcaaaacacaagtaagtacatagcccatggacccaataaagcaactacacaatcgagactacaaagcaaccagctaacaacagcatgacaggaacgcttcacctaacagacagagagtggcaaattggagagaaaaacaaaacctaaccttctactgtcttcgagattcatctcacgtgtaatgacacccatgggctcaaagtaaagtgatgcagaaagatctatcatgcaaatagaaaacaaaaaagagcagggggctgctattcttgtatcagataaaacagactttaaaccaacaacagtaaaaaaggacaaagagggtcattacataatgataaagggttcaatttaacaagaagacttaattatcctaaatatatatgcacccaacattggagtatccagattcataaacatagcacttttagacctaggaaaagacttagacatccagaaaataatagtgggggacttcatcaccccactgacagtgttatacagatcattgaggcagaaaactaacaaagaaattctggaaattctggacttaaacttgacacttgaccaattgaacctaagagacatctatagaatattccacccaacagctatggaatatacattcttctcatctgtatacgaaacatactctaagactggcaacatgctcagtcataaagcaagtcttaataaattcaaaaaattaaaattatgccaaacatattctcagaccacagtagaataaaaatagaaatcaataccaggaggaactctcaaaaccatgaaataacctggaaactgaacaacttattgctgaatgacttttgagtaaacaatgaaattaaggcagatatcaaaaaattctttgaaacaaatgaaaacagagacacagcataccaacaacatatctgggatgtggcaaaagcaatgttaagaggaatgtttatagtgctaaatgcctacatcaagaacctagaaagatatcaaattaataatctaacaccacacttaaaggaactagaaaaacaagaacaaactagttccaaagctagaaaaaaagagataactaaaatcagggcagaattaatgaaattgagacctaaaaaatcatacaaaggatcaacaaaatgaaaagtcagttttttaaaaggataaacaagatggaaagacctctagctagattaacagagaaagagagaagatccaaataagcacaatcagaaatgacaaaggtgacattgcaactgatcccacagaagtacaaaccttcctattgagtactatgttcatatctggttgataggaccaatagaaacccaacgtcagtatcacacaatatacccttgtaacaaacctgcacatgtactccctgaatttaaaattaaaattaaaattaaaattaaaaaattatcaggccgggcacggtggctcacagctgtaatcccagcactttgggaggccgaggtgggtggatcacgaggtcaggagatcgagaccagcctggccaacatggtgaaaccccgtctctactaaaaatacaaaaaaaaaaaaaaaattagctgggcatagtggtgtgcgcctgtagtcccagctatttaggaggctgaggcaggagaatcacttgaactcgggaggcagaggttgcagtgagccgagatcatgccactgcactccagcctgggtgacaaagtgagactctgtctcaaaaaaaaaaattctctgtgttcccttctgttgatgaaatggtattatagttataattatgtatatttgttcattcaactaaaattctacgtttctgagtagaatatctaatcatatttctatttgccttataaaatttcttcagataattgcaccataatttactatttgtgcattgctgaacctttgcttgttaatagttttacatctgtaagaagcaatgcaatgaacatcttcacatacataacatactcctgggaaaagtccactgcagaataaaaagtgaaactgcaagggcctctaaactcttgtagctgttgtagctccactttgaagttgagcagcagtatagggccatcttgatgtagatttttagtctttctaaagggtttttacacccgtctcttctattgtcatctctgactcaggataggtattgtgacagctccatgctataggtgaggaaactgaggctcaggtaaatgatttgcccaaagtcacaaatcacacagcctcattctttctgttttttttgtaccaattaaccatcctcacctgcccctgccaccttcccactactctttccagcctctggtaaccatccttccactctgtatgtccatgagttcaactcttttgatttttagatgccataaataagtgagaacaagtaatgtttgtctttctgtgcctggcttatttcacttaatataatgaccttcggttccatctacgttgttgcaaatggcaggatttcgtttttttatggctgaatagtattccaatttgtatgtgtaccacattttctttatccattcatctactgatggacacttaggttgtttccaaatcttggttattgtgaatagtgctgcaacaaacatgggagtgcagatatctcttcaaaatactgatttcctttcttttgggtatatacccagcagtggaattgttggatcatatggtagctctatttttacttttttgaggaacctccaaactgttttccatagtggttgtactaacttacattccttccaacagtgtaggagggttcccttttctccacaccctcatcagcatttgttattgcctgtcttttggatataaaccaaaaaatttttcataaatataatttgggtggctgcacattattctatttttcaaaggtatcattatttatttaaccattctgatgttcgtttcaactttttctctattaaaaataattctcaatgtaattgttgaatctaatttctgataattttttaggttagttccttagagacagcattactgagacaaagagaagaatttcaaatgctatccgtgtgtgttatcaaattaccttttggaaaggttatgccaatgtatatatccaacagaaataatgctgctattttaaaaatgacattagcacttttttccattttaaaattttattgtaaattgacatttattattgtatatatttatggggtacaaggtgatttcatgatttatgaatacaatgaaaaataaataaagctaattaacatatcaattacttcacatactttttttgcagtgagaacatttgaaatttactctcttagcaatttcaaaatgtacaatgttctattattaactatatttactatgctgtgcagtagatctcaaaaaaataaaaccaacttattcatcctgtttgagactttgtacgctttgaccatcatctccccgttgcccccaccccatagcctctgtaaccaccattctactctctgcttctaccagttccattgttttagattccacacataagtgagaacatgtggtatttgtttttcggtgcctggcttatttaatttaagaatgctgttgttttgatgctggaattaatactgttctcttttcccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggtagtggggctgatacttacacaactgatactaaatgggagacaacagaggcaggctgaagtttaacttgaattctttctctgcttttctgagcacagaaaagataagttaattctccttgaactaaggctgggagtccaggagagcacatttgggttttcccaggaaaagaggattgccagcaggctctaacttgaacctgtggacaatctgctttcttggaaatatcatgcatccattggcgttgtatggtaatggggatggttgcacccatatcaggaaagatttggtgctaagatttttggaagatggacatatgaatcacacattgatcttcccctccatgtttcactcttgtgaatgagacagaataggtgaataaatacgcttggatcgaagggagaaagtggtacatgtcgtaagagagacacagggcatgctctatggagaactggaagaaaactgaccacatttgcaataggagataggatcagaccgtgctttacaagtgggatttgaattaggtttggaaagacaagaaggattcagatacacagagtcgggaggaggacccaagctgtgagaacagcaggatcaaatacagagaggcaggacctgacctgcatactgaagtcggcaagttaggctagaatgagaaataagtgaaggagagtttgtgtaatgtggcagaatgagcacaggcttcagaatcctaggtgtgtcacttaatgactatgcaaccttggacaaggtatttaactttctttggtttcagtttccttattttataaagtagaatagtaattcccaggttgcaggcttgtgagagccttaggttggattccctagcttgaaaaggagatcgttttacaagtgcttcattgaggagagctctgaggcagaggggaatgagggaagcaggctgggacaaaggagggaggtaagtaagaatgtgatctttactggaaacttggcctcagccttatcccatgaagctctctggagcataacttgagccacagactaggccacacctcaagacaagggggctagccttttatatcaggcagtctttggctgtggactgccccagagtatgggggagcataacccccatttagccaaaggcagtgaagggagcagctgtaggcccttatcagctgtgactcaggcagctaggggatggtggataaagtcttggcaggcccgcaccagcattgactagggtagggattggatctacagtccttttctgtaaagggccagatagtaaatagtttaagctttgcagatctctgttacaactactcagctctgcctttgtgacatgaaaacagccatagacaatacacaaaccaatggatgtggctgtgtgccaataaaaatttatttataaaaacaatggctggccagcaggccatagtttactgatgccagtactagagtgaggcttaaaaatgagaaagtataagtaagttttcaagcacagaatcttacatacagtaggcggtaaatatatagtagttatttttataattatttcaaaagagaatagagagaaaccaaattagaaaagtaggttggggttgtgaaaggaaaatagatttactgagcatgagatgaatacataactgaatatttccttaccttccccctttctcttgcaatgtgtgaattaccatgccctcccactatcctctccagctcacttttctcctataaatgttgaagccctcaaaattatttttggagaaaggcacaaaccacagactgtttctataaattctgtgttcttttcttctgggcatgttcttaaccttggcaaaataaacttgtgaattgattgagacctatctcaaatagtttttggtttacagggtcatattggtgtataaaaggtggctgagtttattcttgagcaatgaggaggcctcgcaggatattggagggggtggtgacatgatccaagtggtatgaaatacatttaggaatctactgatgtatgacccctacacccatctgaatgaaaatgctcttgctaagttaaaaaggaccaccatctcattgccaaatgcagttcttatcccactgggctatagcagcattagacaagatttacaagttgtctttcaaatgctttttttttttaaccgacttccacattgtcattcttttccagatttccttgacctctctgattgtactttctccgtctcccctgaataatcttcctctccccactccttaggtacttggaccttctaaaggtttatctttggtcctcttttcttgcattctattcctgttccatggcccatcatatctatttcccaacttacatgctaatgattcccaagtttcaatttgcagccccttctactatttctctatcctggcaataccaaacagcgtgcagtgtcctgaatgccacatttccacccccctgcccctaagcctttctcattgagcactagaacgtcaacttccatgtcatctaccgggcaaactcctgcacattgattaaggtccaaattgctcattgtatcctcagtcatgcccttcttggtgcttctagggaactgtgcctacattcactcatttttacgctcaaggctgtgtgtactgatcacttatagagtacttgcctcagagttcttgtagcggagaactctgcttatctgtcttcctgttagtctatgagcaagagaacaggattcagctgtgcccattgctttatattgagcaactagtattgtctctaggacataagaggtgggtactcaagattcactggtgaataaatcttggaaagatgattcatgctaccaaaaccccatacaactccactgtaatggttaaatgaaagtcagtgagattcattttcatcttgtccattttcatgcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatggtatggacattttctcatggcttgttttggaggttctttaatctgatatgggaaaaggtatttattatgtaggaaatatttactttttgccacataaggtatgaggtatttatgggtaactggaaattactactggtgaaaaaatttaagtatatatcagaacatgtcaacttttggttttgtattattgaaaagggaataagagaggtaactgaatgaatggacttgggatcctgtgaaatatctaacagaagtttttttcttaaagatgatgaaacagtctcacagaactgaaatggcttgtccaattagtgtcagcattctaactagacccctctggttctaggtaaaaacttcccaaaactttagagttggaatatattctaaaaaccattttatagaggagtaaactgaagacaagagagggaaagtagcccgaagtcaaaacgcaatgtagtggcggaaccagaacaagagccctaatctaaagtcttttccataatattatgcaaagcaaagtgtagctttaaattttggaattcttaaagaacaggtgtttaatatcaagattagaagggattgaaacattttgttcatcctgctgaagcgtgaatttcccctgtaacatccccttagatctgtgctatccttcaacatttctgcctgaggcagcccatttccttttagatgccttggacttgatggaacatgacaaatgttcaggagatccaatttcttcagtgaggatgccatggcttttgattcttcctcataaatggaggacaaatagagcctaataccttcttctcttctgtatattctctcccctatttttagttatttgtgacataaagatcagctcctctgtctggagcatttggtcgatgaggtcaaggtcatgagctccatccttgagtagacaattcactttgccttgatctctggttccagatacttggtgatcccacaagggcctgggagtgagccagtgtaaagcctatcatcatgtttgaaatagttactgtaagcacagtcctagtggattacagaccagtagcagcatcctcccatgaagacagtaacaaacaaaggtagttaacagtaaccatacctgcttcctaattttaaaatatcattaattcaagtcaatgaacagatattttattatttttaaatattccctgacagcaggtaaaaatggttgatgactatgtttcttaagatgatattagatcagtgatgaaataatctgtacaacaaaccccatgacacgagtttacctatataataaacctggacacatacccctaaacctaaaataaaagctttttttgtttttttttataaaaaagatagtattagacatggttaaaccaccggtttggaaaactcctgctttttttcttgactctgcccctgatttgtgaataatgttaaataaattcctttattccttccattcctccctccctctttctctttctttcttcttccaaacatatttatcgagcacatactgtgtgcaagacatttttctagacactggagacacagtgatgtgaaaagtagataacatcccctgcttacatttcaatgcatttccttcatacactagagatgaactccactgcctctttatgtcactaataaaggataaaatgaaataacatgatttgttaaagcattttaaattcttggtaaggaagatatccttacatttaaagtacattgtacttttcaccatccagtggatagtgccctccaggagggctcttttgtttcatttcccctccctggtttacaggcttttcactgaatctgtttagtatttgtttatttaacaaagactcacagaaggcatcacgtagcagacatgggactaagttatacagtcttaaatgagacattaacagtcactgacttcatggagctttccagaataaacagtgtgactgggaaagtggatgaaatagctctaatgaactctgataagaggtatttaagggaggaaactgaaactaatgaagaaaatgaggattagaagtaactttgggttttatttacctgttctgttccagggcttcaacctactccaaatcccttagctgagaaaataaatatatacatatcattatgaatactgtcaatttttctcatgtatccttatagtaagtacatcttgaggaataatattttgaggttaggaaaattctctttaacacaaaacatccactgtcatcttcatcgtaatatttatccttttctattttactttcagaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagtaagtcaatgaatatgcaatcagtaaaatgttttctaatgagtttgctgtgtatttaaaagcatttgactggcgtgtacacacacacacagaggcacacattaactaactactttttgtatttttagtttgttatattttatattttagtctccatttggataccatctcctcagataaaatttccttgggtcaggtgacactacttcatactctatattcttaagtattggcttactcattaaactatgatattatgttgaaagtgatcaatttattcattttacatattcttttatccagccttttaaacaaaacggtgaccccacattttaggcaaagcactggggatccaatgttaataatttcaggtatggtcctcaaccccaaaaaacgtttagtttatggcaggatgtggaaaattaaaccactgcaatttgtagtgttgtgatgggagggtacagggtccgttagcgtccaggggagatccaaagcccaactgggggtgtcagatgaggagaggcatttaagccctgagaaaggtgtgggtgtagaggggaacacagagaattccaagctagagagaacctgacacatttgggcaacagtgaccatttccatggttgcagcactacgttacttattgctgtgtcacaagtgcctcacactgtgcctgtcacatgcaagtatgaaataaataggtttttggaggaaggaattataagagaaaaagaaaaagaaggaaaaaaagaaaggagagagggagggagagaatggaaacaagaaagaaatctatcttctttttaccatcatagaccctggtcaattatttctttctttatttttcttatattttctttttgaagtgaagaactcatgtgtaacaagcagttccacatgcacaggtttaaaacagtacacttcttctgtcaagattctctctgttaacataaatcgtacttgggaggaaagacgcagcacctataagaactgttgccagtgtttaaatattactgaagttatgtctacaatgatcctgcaattttaaaggaaaaatgtattccatttaatcaaagccaaatacatgattttgctacctttttttctggttggattaatcttttgacccctcttagcttattttaaaggcacatggaatcagatccttagagtgattcattaatgcatcaattgttcctttgttcagtaaatataaattaattctttctgtagtgtcaggcaccatgtttgacactggaattagagagaagaccaagacacagccccaccctctaggagctcaagtctctagggcaggaagttactatagttgaataactacaaaacggggagataaatggcaaaggaggcacaactagagaaggctaaaggagattgaggggttaattctgccagaaggtggatgggatcagggccaggcttcacagaggagggaacacttgagctaggtaggcgtggagaataagtaagatttcaattttattaaattctctctgccaggagtacatgaagtgtggtgcctgagaggaattattagggctttcactgtacatttttggccatgcaatgcactttatgaatgttattaaattagtcttttgaagacatagctattttccaagggtatattctggctgaattaattaaaactaagagataactacataatgagctttactgtctgattttttaaaaatttttatttattttttatttttgtaggtgatttttttttacattgtttctatttcacccaacaaccatttctaatgtaaataccattgattggctggacacggtggcacctgtaattccagcattttgggaggccgaggtgggcagatcacttgaggccaggagttcgagaccagcctggccaacatggtgaaaccctgtctctattaaaaatacacaaattagttagatgtggtggtgcacacctataaaccaagctacttgggaggctgagatgggagaatcgcttgaacctgggaggcggaggttgcagtgagccaagatcgtgccactgcactacagcctgggtgacggagtgagaccctgtctcaaaaaaaaaaaaaaaaaatttgattgatgaaactgcactagttatgcccacctgcttgtgatgcttgtgatgggtgatccacagctaatgtattgttttcttttctccccaaaggcgagagatagttggggtggtggaacctgtgccccatgatgaaacatactgtgaccccgcatctctgttccatgtttctaatgattactcattcattcggtaaattacagttttcttgtttctgtttaacttctagggtttgtggacagctgtgctaataacgacaaaaagtagggataaatgaagtgataattataatttttctatttccacccataactaccaagctcaatacagatgctccttgctttacaatggggttacatcccaataaactcatcataagttgaaaataccttaagaatgcgctgaatacacctaacctatggtacaccaaagcatagcctagtctaccctaaacatgctcagaacactcacattagcctactgttgggcaaaatcatctaacacaaaccttattttataataaagtgttcaatatctcatgtaattatttaatactgtgctgaaagtgaaaaactttgggtacttaccattaacatatacagctgaaagcgctattgtaaagtcgaaaaatcaaaagtccaaccgttgtaagttgggaactgtctgtatagatgatcaatttcaattcaatttaaactaacacaatttattgggttaaattaaacttgtgtaagatcttgtccccgtcatcagcaatcaatagtatcattgggagaaataaaaagtagttttgtcttcttgttactggcagtttattgtacattgtgatagagaatttttaacttgaagtccaaaaacatatgttcttcacctagtaaccccagtccttgaatttgctggagctcagtttatttgtaaaatgaaaataatattatcgacctgttataaggatggatttgcatcatttatgtgaaagggctattaatctgtaaaacacaaaacaaatgttagctaacatttaacagagtaatattgccatgtttatcaacagatacataaaacacaaataggttatgatgagtgtagagttcatgtctggagaaagagatttcagagatttgatcagtatctctttggttacttgggctccagatttaaatatatgccctaatctataactctaagacagtaagtaaacacgggactgcttttttgttgctttgtctcctgtgcagatattacacaaggaccctttaccaattccagtttcaagaagcactttgtcaagcagctaaacatgaaggccctctgcacaaatgtgacatctcaaactctacagaagctggacagaaactgttgtaagaaatacctcaaaatgttgaacctctcctagtattcagtattactcatttccatgcctaggtttgtatttgatttctttgttctaaaaagaaaattttatggcctcaaaatgtcctcatttacaaaccaaacatttaatttgtggtcagacaggaacctagaccatacaacaattgggtgggccacctcttttctccctatcataactacagccctctcttcctggtaattggaaggaaagagcggtttagggtggaatatatctgttaatatgcattcttttcttatctgccagaagcaaatttagccaagtcaaagagaagaaaccatagatcatagatgtaaatatatgtacatctggaacccctcaaaaggccctgaaccccctttttttgtgtagcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatgtgagtacacccagttgacaagtttcacacccattccttgtctacttcgtctagtctcctttggccccagtctcatgaggacttgtgacacagctgagaatgtttctttctctatagtacctgcttctttctctgctatggtgacagctgccattttatgggagaaaaatcacaactgttgagcacctatgtatgagacatagtgaaagcaactgtacagacaaacacacacagatgcatgcacatgtgtgcatacacatacaagagtgtgtactctttcaagtctgttttattagtctcatctttaaaatacatattataactgtctccaaccaactgtactcgaatcctgctttccaaagacaaatactttttttaaaacaattttaatgctattatctcttctttctggcagtcactttcatatttctaaataattttcttatattgctattttgttattgctcagttttaaaccttcgttttggtctactcttccacttccatttcttgctcttccaataaggccttataatgatttttagtaaaacagttatcaatgtttacatcactatgcctgaagagccaagtaatgtattattttgtgtcctttcttatgtggcttgttgtttgtctcaaagtttctacttgcctgaatttttcggcttacctaatttgctatctacgttgtatttgtttttctcccaaatatttcaacatatctattcttttcagtctcattttttttccctagagacttccctcttggagtcttatattctcctttcccaacatttgttgttctcctgggttgaatccactgcttcctagattccatgtcttctttcttggattacctcttgtttttctggagcatagcctcccacagactgtccaagcaggggcacaaggggtaggaggatcaactgttctccaaagtggttttaaccacaccctgacttccattcctcttcttttcagcccagtattattttcccaccctcacctgtgcttgaattccaaacctcttttggattttttggctctttgtgggtctgtgcctcctctgtgtacaaattaggtcatggcttcctgtccctgagaaagcagttacccctgtctcatcatttctcgttttccaaaagcctgttgatacctttaaaagatccctttactatcactttagtgggatctttggaggaaaaggaaataagcacatgtgggcaatctgttgtctgtaaccagcagtctgctatttctctttaatcagatgcagaccaaagcatcaaagtgaggataagcctaaaatcagctcttggagataaagcagtgagtattctggacagtgaattgattatttttgagtgcacagtcttcacttaaataggacacaataaacccatttcagtgtgaactgaaagaagagaggccatgtggttcttttcaaaactataagttccagattctggcctttggctttgggtttttaaagtaacctattccttattggataagctattcctacttcttattgccttgaaggtgttatgaacttcaatgagaaaatatctgtatcaatccttaaatatcagataccaatcctttcaattgactctcgtcaccattacttttattctagaaagagaatatggttatattttgaaaggtagtttgtttttagaagcggtacagttaggtttttttagtttgttttgttttagtcaatcacctgtatcctggcttcactcagagtgtggcccatggtccagcagcatttgaatcacctggaagcttgttaaaaatgcgtaatttcaagcctagccctggaccacttaatcagaatctgcattttagcaagcctccaggtgatttgtacgtacaatcaagactgggaagctctaatttagaacactgcttctcaaacttggctgcacagtggaatcacatgggaagtttaaaaaatattaatgcctaggttatacacctccagagattctgatttaattgttctggggtatggagtcaagagttgggcatcaagagtgtttaaagctccccaggtgattctagtcacagcaaagtttgagaaccacttatttagggaaatgcattttgggggtaacaaaatattttacaggaaattatgttatttacaccttaatgtgaattatttacttagctattgctctgtcgttaacacctgtctcagatgatccagtctgaggaagtggtagctttgattgggaaagaggagaggaaagagtagtctcgggtagatggggctcaaaagcctgagagtgggtatgacaccctgaaggctttgcagggttagaagtcaatctgggactattggataaaatggtccaagatgctacagcaaattgttgtctgcattggatagatccaaagattgtgtttctacccccttacctttcctgaaaagttaaacattgtccaagattcaataacacttttagtataattttcatagttaagacatcaaaacataaataactcaggagtaaaaagatgaacattcaagaaagaaaaaatgaaaatggaagtgcatttctttccaatcattaagagttcaagttcctatttcataataatcattgggtgattgtgggactatttctctgtcatatttttattagctcttccttggcttaatacattttaattagtctcggcagatcaggatattttcaattatcttttttcttgacatttatactcatcctctttccatgcagtgagggttggtaaatagtgttcagggggtttgattctgtaatgttctgaggccaattcactgagcaacaatctgttttgatgaaacgagatcatatatttagtgcatctacacaacagggtcagaatatctgctcagttcaaactatttcatggtgaattccaatggatctatggcattattttgagaactcagcctttaaaatcttttatattttatatttatagagatagtagccagactgttattagtaacaatattaataatattattactattaaaggaactcctctaaacctcggtgaaaccaagctgaagtgaatgtaacaattcttttgtgaaatattataattttttaaagggaaaggagaaacatacaactagtgccattcattgattcctactgccagctactggaggaaagagtgaatgctttcttttaagtctttaaaagctatgcaaacataagctatttgtttagcactggaaaacaagaacaaacagattagcttgctgtttacaaagtgttatttttcatttgaatgtcaagtttttcttttacacttatagataagtacatttctgagaacgtaccataaaaatagtaaaaactgattttgtccacaaggtacagacattttgctacaaaacccacaacttttgtttgactcccatcggtaaacctacactcaaggtgttaatttcagtttacatcaaactaaagtaaaaggacccgtgaacccaatggttcctaaagcatggttgcctgaccagcagcattaccaccacctgggagtttgttagagatgtaaattattgggtttcatctcgggcttcctgaatcagacgctctaggggtggacccagcactcagtgttgtaactaacccttcaggtgatacggatgcacacttacattgaaggtcactgacttaatgaatagcaagaatctctggaatattaagaataattcttgaggagtatcagattataaatgtgtcttacgagtccctctgagcagtgtctttcttctgaatttgcagtatgaatggaacgacaatgaaatgtacctgttccgatcatctgttgcatatgctatgaggcagtactttttaaaagtaaaaaatcagatgattctttttgggtgagttgatttgctgggttctcaaattactccaactaggatttctcttactcttgagtctgaggagatactctgcctaaacttttcttcaagtgaaattgataaaaatctcctagtaacttatacttctttggtattgtggaatttgggtgactccatagagggctgatcgtgccttatttgactggtaaattgcaagagtattgttcttactaaacagctgtcactctcttctcagaatgctctcatacaccctcagcactgaaagaaaaaaaaggcagaacttcctgccttcattgacctatattctagtatgtatgttgtgtatgtgtctatgtgtgtaatatgtaagtgaaatatagagttacaaggtactaagtgctaggagaaaaatataaaagggtctggggagggctggtgtgtgtgtgtgtgtgttgggcagggggtaggtcaattttaaccaaaatggtcagaaaagacgtcactaatacttgaacaagtatatgaagcaggagagagggagagagagagcgagagagcgagagagcgagagcgagagccagcaatgcagacaatggggaaagtgtctcagctgggggaaacagcaggtgcagaccctgaggaaggaatatcctcagcctgttaaaggaacagcaaggaggccagtgtggctggagcagattagaagacaagtggagacatggccaggacgatgttagggcatagaggaaggagggagagacaaaaggtcatgtgggccttgaaggccattctaaggactttggcttttattctgagttagatgagtagtcactcaagggctatgagccacatgatctaccttattttgtgctaggatcgcttgacagctgtgagagcacagtcctgtcacatcatatggtagaccccactcattaccttcatccatgggtgcaacacgacgttttctgctgggtaggaccaggttgtgggtgtaaatccgtgtgtggtactcagccatttctgcccaccacattgtttgccacaatcctgggccaccgattattccttgttgattctcctgctgttttcagtttggaaattttctataattgaaaaagatgtgggttttatataaaggcagaacaaatagtgccaaaacttgatatcacaatttttatgtagttgatttcatgtgctttgggcagcattaatgttttttttgacaattacatcctctcattgtttgccctcccccatgtctctctatcctgtgattctctctctaatctccttttcaccagcaggggaagtcttcattctcttgattgtgtcctctgtgccacaagtgaagatgtttgttttgtttctctacagggaggaggatgtgcgagtggctaatttgaaaccaagaatctcctttaatttctttgtcactgcacctaaaaatgtgtctgatatcattcctagaactgaagttgaaaaggccatcaggtgacattttactttcatctaagggtgagggggttaccaaatacaacaacaataaccagtattttgtgtgttcttcctgtgtgccaagcattattctgagtcatttacatgtgttacctcatgctacaaactccacgtaattccagaaggatcccacagcaggggaataatcttgatttgagttccgccagatgtaggctcttacaaaagggaagaaaaactatagattacatgttatcaagcaagttaccactatgggcaactagagctcagtcctgctgggaaatgtggagagactgtgtagaatgtgcacctgaattgtcccatcagagggctgaggaagctggggtatttatttaccacctcccatcagttattggctgagaactgcttccaggtggcattaattccccagcatttcagccctgcaggcagggcagatcttgtggtctgagaatgctctctggtaaaggggagcaggtgctggttgaagaaagtctggctagaactcatggaaaatagtaagtgcagaagggacgtggacaggtattgccatcatttgctctcaggactttgatgtaggattcatttaactgcccaaatcctgaaacaaggtaggcaagtacaggattccttacatattgcttggcctctatgctaggtgagggacttgcaatgaatgtttagaagaaacagtttgtccaatttgcctcaattgtttcccacaacagacagcaggagcgaagaatggattgaccttgcagatggtttcctgtcattataaaaaggcttaatatacctactgcttctgggcagctcactaggtggccctctcgagctgcgtcagagtcctgtttaacttcggaagcagatctaagacctgctgggcagggtgtgcctagaaaatctggcttggttggagtgtatgacccaactgtgcccagagactcactcacttcctccagaagacttgtgtctgtcttgggaaaaacttctggctgagatctttatgaaaaagctcagccttcctggatactcagctgcaagctacaggggacctcagactctctgtttccaggatgactcttttcagatcttttctggctgaggttgccaggggttacctgactaattctgcgttgatctctctctctctctctggtggactctgacctcctgacctgggctatacccaagggtaggaagatgagatgccagacactttggtctttgatccagaatgttctccatatcattgtttttttttttctttaaacaatctttatccttttgcctccataaaagtaagccatttcccatcccagtgctgaagaaactggcaaaggtccctttcttatgtgcctccccagtgctacctccaaatgccaataccttttatttggaaaatactactatagagacttggtcataggacctgattcatttgtataatagaagaggtgtatccaattatccgtttccattatttgatataaagtcattgtagatgtactctgacagaagggaaattcttgccaaatatgataactttgcccttaaacacagcagtcacaaatgaataaatgccaaccatttatacatttccacacttacaactcaattttccaatggagctgttgatgaacctaatctaggttgcaaggcatgaaagatgcataattgtcaaagacttatatctttaattagacctatttactttgctcttatgattttaggtgtatattccttttttttttttttgaaacagagtcttgctctgtcacctaggctagagggcagtggcacaatctcggctcactgcaacctctgcctcccgggttcaagcgattctcctgcctcagcctcccaagtagctgggactacagacatgcgccaccatgcctggctaggtgtatattcttatgataagctctattatatcctttcaggaacaatgatataaacagtaaataactacacacaattttattttgtctaagtgtcccctttgctgttttttgcttttgcaaataggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagtaagtggcctttcctagacttaactatccaaaaatagaaagatttagagaacagtaactggaaaattttcagtaagtggtagattgtatagaccaattacttagttggacaattttgcaactaaatactctgatgcattccctagaaatttaatagatcagcagttcatcttcctagtaagcccacacagtaagtatctggcctgggatttattagcatgccctagtaaactccatcccagccacagtgtcatttccctcctacaaagaggcactgcaaactcagcaggtatgggtgggctcagggaatggttttcactgctctcactgctgaatccttttcagtctaaaagtggggactggattgctggagactggattgctgatgggtttgaaccgtgaaattgttcaagaatgggctgtgagatttctttgaaactgctactcttggttttttgtttgtttgtttgtttgtttgtttgagacagagtcttgctctgtcacccaggctggagtgcagtggtgcaaccatagttcactgcaacctcgatttcccaggctcaagtgatcgtcccacctcagcttcctgagtagctgggactacaggggtgtgccaccacacccagctaatttttgtattttttgtggagacagggttttgtcatgttgcccaagcttttcttgaactcctgagttcaagcaatccgcctgcctcgccctccagtgctgggattacaggcgtgagccaccgtgccctgcccaaactgctactctttactcagctttatttctcaccgggtagctcagactctatgtcattctcttagttcatggttaggcacgttctaatttgttcctgaactgaagatcaaagttcattgaggatttaaattaccagagacctgatttctagtcttgctttgctactagatgtttttcttctgtccttaagaaattgactcttgctgggcttatggaatgaaggttaatattttatagcacctaacaaaactctatgcacacctcagcttgtttaaacatattcttgattgaatctctatatttgctagtctattttgctcaattttcttcactatagagatttttcgtttctccaaaatagctccatctaaaatattaggcattcaatgtgtaatacattcatatggttaaaattaaaaaatacaaaggtgaacatgaagagtcttccttattaccttttgcctccaagttatcttcccaaagccaaacaaaattatcagttccttggagatacacacacacacacacacacacacacacacacacacacacagtcttttttcttttttctcagacatatatatatatatatatatatatatatatctcctttttagtatacaaatatatacttttttagtgtgtatatttgtgtgtatgtgcatatgtatatctcatttcttagtatacaaatgttagcaggctataaacaatgttctgcatattttgttttccacttaatggtttttttaaatttttatttatataggtgtgatccaccacacccagcccacttaatgtgttttagagatcattacacattagttgcacaaaaaacttctccttttttctggcagtataatttcccatttcacgaatggattgtaattttgataaacagtcatctattgatgaacttttatgttgtttccaaacatttgtgtttacttaaaaaaagtgatggcttaatataggtaatttcctgagtatgccaatatacccttaagatgaatcctagcagtgggaaagctgggtgaaagcatacgtgcaattgctaattttgataaatattgccaaattgccctcaatagcggctgtaccaatttatactcacatcaacagtttatgaaacttcctgttcctccctcccagcctccttaacacagattcccctgaaactttttgattttcatcagtccgattgattttaacataagtatattaaggcaaccttgctctatttaacaggaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactccttgtgcttacttatgtgctggggcttctttacgttttgtctgcttttcagttctatgaacaaagttatcatctgagtgtctggggctcctggccttccaaatgtctcgtgatgtattgcaaattctcagattgctttattgtacaaagtaattgtaaaactagtcccaaaaaagttgtgtacagagtgtatacaccatcaggttaggaaccaaacctgatgctgcttttatatttctcacagtcactaccccagtgtgagcacagcatttcttagatacctgaagactggttaaaactagattaaatagattaacatatctaaactctatgcattaactcattctatgtggtgaccagcataatgttacaatgactttgtacttcataaattctctatggtaattgtggatcagttagccaagtgaagggaagatgactttcataatctgtaaaataacctcaaaataagggtcatttgaggctctttgatgtcttaattatctgatacccaggtagcagagatagagttctttgttattttacgagatacaatcaacatttcgcatcaccatctgcaccatcctgccattcactgggcccacagcttggggcaacatacaatataatttactccttggctaagcaaccatttcatagacagttttttcttttatcttgcatatgtcccatcttctctaatatatcatgaaaatcttctgtcaagctactgctatctaaacatcctttgtagttgccacaagatctttatgaagttcataaacttggttcagcacagtgcaagaataatttctgcaggaaccaatttcatcatctaacaccttcaaaagagcctccccagacatggcaaaggacccatgtgcatgtgacatagtgtccaactgtaacacagaaagaaaaacctgcttatttccgtaacttaattttggggttttctccagaacagtcctcactgtgcccaccaggtaacgcttgtggtccttctcctgtgtagcagcatcctctgaaaggatggagactctacaaagagggaccggtgcctgggcctaggatggaagctggcaaacaatggcccatgggccaaatcctgcccaccacatttttttgttttgttttgttttgttttattctgttctgttttgagacggagtcttgctcggtcgcccaggctggagtgcagtggcgcgatcttggctcactgcaacctctgcaccctgggttcaagtgattatcctgcctcagcctcccgagtagctgggactacaagtgtgagccaccacacccagctaattttttgtattttttagtagagatgcagtttcaccatttggccaggctggtctcaaactcctgacctcaggtgatctgcctgactcggcctcccaaagtgctgggattacaggcctgagtcattgtgcccggccaccccaccacctgtttttgtctggcctgtgaactaagagtgatttttacatttttaagtggttgaaaaaaattagaataagatttcatgataaatgaaaatgatatgaaattcaaatttcagtgtccataataaagttttattggaacccagccacacccattcattttcatattgtctagggctgctttcatgctacagagcagagctgagtagttgaagtagagacggcatggcccacaaagccatttacagacaaactttgccaaaccctgggctatacggaagcaaggattaaatgaaactgcaaatgcaaaaggtccattgtggagctcacagaccagagaaacatagtgatcaggccagaaatggcaggtgctagcttcctcttctctggtgtctctcaatgaactattcttcctaatttgagagcctcctccctccacatttaggtaaagatggggccttaggtccaatctctcctcctcataaacccactctcaagatagaataaaaactgagaaactcaacttgctaaatctactgtgttggaatcttaataaactcaagtagatatgaggtgagttttgccagaccctcttgactttgtgtttgacttctggtaggtcagaaaatgctcagcaagacgagggagcatctgttggattttacctagcaagtgatttatctgaacttgctctgcagcctgccctttgggaaagtttcttaccacatagtcttcctttttcttcactgccaaaagagtgtaggcagacacttggtttaagataacaacaataaattctgcatatcaccactaaacacagttatacagcattaaaaaatcattgtgcacgtttgaaatactatgaatagtgttcttttacttggctcctcctagcatcactggacttgttttctgataaacctctagtggtaccttaaattgctgcctgacttgatttttgcagctaggcttctaagcagggctttggcctcttttcatagtaaacaatttttgcaacaagcgtgtattttgttataaagaggattatttttaaaaggtaaatatacaaaaataagaagaagaaaggaaaaaaatcttcgagagggagaccagaaagacccagcttgtgcatgagctcatcctctgagtggggaaacagtctttggtgcaggaaaggctggcacccccatcggactgtgggtccattgagaaagagggtttgggtcacctgttctttcttttctcttgggtaggaaagaacctagcatacagtcaacactacaaaaaaaacccacaaaaataaaatatacaaatgccctagtgattattttgagttacatttaattataaattgagatttgtgtacctctgtaaaaaatagaagactgttaacgagtgtcaattctgtgttgaaatgaaggaattattaatgaaatgtcaagaaacaaataagcaagagggagcattgaaactagctggtggctgaaggacagagtgggatgagagaggatataagtgtctctgagagattattgactgatctctggaaaatagcagcagggtttaattggggtgtctcttcattgtaatggtgccctctacagaatctgtctctcccttcttgacattatagaaaagacaatggaatactcagaaagaggttaaaacctgtgggttcggtagcaggccgtgaagaagggcatccgcaggtactgttttaaaggtgaaatactatttagtaataggcatgagaagctaaaatagtacttctgtatttttgtgttaagatagcagactgaaatagaaacttcagttatcaaactagccagaagccccagatccccaaactcaaggatttaagatgattagacagatggtcaccaaaattagtccacaagtagaagaatttacagcaccatgtcatacatagttcatcctaatttctaccaacttcactgggcaacaatcaatcgttttatttccctcaatgactcttgttatcttcagacctgaaactgattcggagaccatggggcccacaaacctaatcagagtaacatgtgttcattgagtacacatgtagacatgagaatctccactttccccttttttctcttggtaaaatgttcacaaatgttcaggtaacacctgctactccagccattcaggccttaaatctgcagctctacattttgtatccaggtcttgagatttgggaaatagaaaatttttatctaaaaaatgaaagtacttttggttatcaaactcagacattgaaatgaacgtgcagttatgtcgttctccccctttaaattatgtattcatctcttgaaactgttcactattgccacaagtagatatatattaaactaataataccacattggacactatatctcataccctaaaccataatgatatatatctaatcaataaccaatgttatttctttaaataaataaaaatttctgacaaacaactctgtatcagcccactctctgtccctttgtttttgtctttacaactcctcttgcaactgctgctaatcaaagtgtagattccaggcaacttgagtctttgctcccaggttataatccttaaacttgacccaaataaactgtctacttatataaaaaaaaaagatgattagaaagcaacaatgtaattaattctagaaattgtacctcagcatcttaaaaaatttgctttatgtgaacatatttaatgtattatttaatattatttaactttagttttatacatgacataaagagtcattgtactaatctggatattagacgagaattgtgccatttggattgggtaatggaccacctaatgataccccaccatataatgaggtcccctagggcagttcagaacaactgcttcagtaacatctgggagtttgctaaaaatacaaattcacaggctgcactcagacctcctccgccaggttttatggaggtagtttctaggaatcttttcataaactctgcatttgattatcatgcctactgaagtttgaaaacattgaaatattaagacaaatgtccacatttctgtacttggtagtatatttaattgttgtgacaatgacaatataaagaaaagaatgaatatccaataagtaatttttcagttctgctcagtctttacacatgaacacagtctctatattgcagtctgaaaatgtgcttctgctataggctggaaagtgttatatcaggagcagcataaacaaaattactaatttgatctaaatttcaagggaatcttttgtttcctgccttgttcatttctaaataatagacaatacagagaacactattaaaaactagctaaattacattatgccttgctactttccttaaaatgaatgaaaatgctggccagcatctatattcattcttatcagcactcctaatttcttcttcaatgcttgtctttttcccgaagtggttcaatggaagacaggagttgctgaaatgtgggacgcttttctggaagctaaacaaaaacaacaagaaacccaatgttttagatgatgagagttatgatcaatgataaaatatataaaatatttacatctaagtcctaaagctagcaacttttaaaatgtggaaacacaacagacatttcaattgaggtcagaaataaggcaaggacgctcactataataatcactcttaacattatgttgaaggtgttaaccaatgcaattggaaagcaagaacaattagagccccaataactggaaaagaaaaagtctctttatttgcaagcattttagtgtatacctggaaaatctgagagtatcaatgttagaacagacttaaagaataaaggaattcactaggagagcaggatataaaattaacatacaaaatcaatagccttcacatatacctacagtaaccacttagaagttatcatgtaagagaaaaccccatttataatagcaacgccaccaaaataaaacactaaaaaagtgtgcccctctaaaatggggacgactttaaaacactgctgaaaaatgcaaatgtagacttgactatataaaaaagcatccctggccaggcgcagtggctcacgcctgtaatcccagcactctgggaggctgaggtgagtggatcacttgaggccaggagttcgagaccatcctgaacaacatggcgaaaccctgtctctactaaaaatacaaaaattagccagctgtggttgtgcacaactgtagtccaagctactcgggagggtgggacatgaaaatcacttaagcctggaaggtggaggttgcagtaggcctagatcataccactgcattccagcctgagtgatagagcaagactctgtcttaaaaaaaaaaaaaaaaaaaaaaaaagcttccctcgttctagactaggacaagtcagtatcataaagatgtcaaatttcccaacttaaaatacaaactaaatacaatctcctcaaaaatatgaatgagtttgtttcaagctaaacaaattgattctttggtttatagaattaaaaaatatgtaaaaatggctaagaaattatacagtaaggtgagcaatgaggggtgctagcctacctagtattcaaacatatcataaagtctctacagaatcagagcagtgtggtgctagcacatgaacaaagagaccaaagaacagaagaggaagtctccaaatagattcaagtacatatggaaatttagtataatattagggtggcatttcaccaccattaaggtggtatctcattcctgatgataagatggtcttttaataaatagggctgccaacaacagaatagtcacataaaattatatatttggagccatatttaataccatataccataacaaacacaaactatatcagaaacataaatggaaaataatgcaatcttaaaagtactagaggaaaatcctggcaaaagtcattataatttgggcatgagaaagcctatctatgacaatttccagaagcaatgaaaacaaatattgatatattcagctatattaagaaaaatttctgcatggtcaaaaaaagaaaaaaaaacccacccaccctcaatcaaataaagctgataacttaaagtattactctccctaatttgtaaagagatctttaaaacctgacagaaaaaagaaaaaagacatgacagttcagaaagacacaaaatgacccttaaaaacgtgaaaagaggctcaacctcataatgaaagatatatatattaaagctgcactaagatgccatcaagctttcacttatcagattggcaagaattcttcaaaagcttgacaatgcactccattggtgaggttataagaaaataggtaccctcttacattgctagtgaggcaacgcctatggagaggagaagttcatatgctactttttctttgtaagagagaaagggaagcaagaaaacacacatctatacatatgcttgtttttgcatacagaaataccagaaggatgaatcaaaatctaatgaaaattgtgaatgttctttcaatctctttatgaattgagaaagtcacaaaataaaatctaaaaaaggagggaaaacccaaatctataagctctgctcattaggtgcaatgttttaactggatattgtcgtctcagtgcaaacccctcttcacagattctgccacaattcacatgcttttatgaaccaaccatgagaagaaggcagttcctcccgactcagggcccctgtccatcctatggtgcagtcatattcccaggagctagagtagggagggccagggaggttctgggcttcaactgaaaaacccttgtctgctcttcttcatctgacaggtgacagcagtaagtcagcctggctgtgaccaactctacccagacccagtctgaccctctagtgctgcctgataatcttagacacaatatttttgtttgtttgtttctttaaaaagcctttattcattttttaatcagggaaaaggacaatctcaatgtcaatttgtcacctaatcataaatttgaacttacaggattcgctaccctagacactgcacctactgacaacacacaacttaaaaatgaacttccttccattccctaaagttagatagttgcatgttgaatgaataaacagaatcccttccaacagctaacattggtacttggcctaggtgtaaggacaaatagcgaaacagcagcaaaaaagaccaagagcattgtaacttgatagcccagcaagtacaatcattgtaacaaagttgtaggcaccaaattaaaaaaaagcgggggctcatcagcataagaaacttacaccagttttgtgagagcactcgttgtgtgagatcaactactctgcctgtgaacacatatgccaatcccagtagctaacattgaggcaaaggtcccaaaatccaactaactacactgaatgcaaaatccaaagcttttatttatccaggtcctctgaaacctggatataaaacagtttcttttcaatgcaccatgagggtccactgtaatgattcagtcacatttggattgagtaacgcacctaagataccttttccactattcctgtctgaatacggaaatgtttaactactcagttcatgcactaactgtgttaaaagttagatttactatcaagactttttctaaactcacacagcattaacagttaactgtaatacagcttcctcagaatgcccaaacagaaaatattttgttcagtcttgtggtggctgtgagaaaacaaaatacaactaaatcttattttgccgctatttcaaattttaatctaacttttgaccaaactcatacctttctgccaaaatccttgagttataactactttcaatgctcttaaaccatagctcctaccagttttacagtgctccttcctgtgtgcatctgaatttttatcaagaattaaagtcatggtgttatgattacaaagctttttctaggtgaccactgattaaagccaaagtaattaacgattcaaagatggctttctagagacaggaacaaagcaaaaggagctactaacaattttattacaatccagaagaaacatggtctactagaaagtttctactaatcacaacacttatgtcttcctctcaggacaatcttcagataaccagaggggaaagaacattattcaaaagtgtggtagaaacctcagttaacagggagaagatgaaaccttaagagcatagaatacatctacaaatcggggaagagggtgaccctggctggctatttataaaggaccacagtttctccctatggaccaatactctctacaaaatcaactgtgccttaattattgcttactttaaatttttacagagctatgtagtacctgatacagaaaagcaatcacataaggaagcatttatttgaagtttaacatgaaatcatacaaataaaatgtgtataaacaaatccatattgagtcataacacagatagcaaaggacaaagtaaaaacaaagaaaactaattggcagccatatacagttggacactgtgtcttgtacactagaaagtcttttacaaaataatcatcttagatcaacagaagaccaatcttctatgtcatcctgcaagatgggttactttaacatctcatcctgttttctccaatgttctcctttagtatggctggtaattgttttggtgattgccaccccctcgagatgccttgccataagtgctctgttggccactgtagtctgcatatctctgtccatatccatagttcccatagtcaacatttggggtagaaaaaggtgtggaaaccttatatactgttccaaatgccatagggtgggaaattacagagtgctcatattccaggtgataatttaaggctaaacgaaaaatattgttatgtgatgcaatcagcaactcttccaaagtgggactctaggctccggtacaagagtttagaaactatttaagttgtgtgcgtcaatttattcttagcaccaagcgtactgcctggtgcatagatgctttgtaaaaaaaaaaaaatatatatatatatacacacacacacacaatattaataacagaaaaacagcactgtgtaccaggcattgtcctaaggcctttataatgctaattcattcaatccacccaaaaattctatgaggtaagcattacattacagtgaagcacagagataataataggaggtttgtacaaggtcatataactagtaagtgatcacgacaggattcaacccaggtattctgatgccagaacccacaggcttaaccattacaccaatttgccttttactagtgaataaatgaatgcagcattaggccctttgccattgaggaagtaccatataatgattatgaacatgggctcttagaataatgttctcaaaagcctccttttatcatgttactgctcaaaagtcttcagtggctcctcatcgcctacaaaaaccaaaactctcacttgtctccttgttattcactttgcagaacctgaaccagacctactcttccaaacttatctccagcccttccaacgatagcaccagcctagcgtgccatgctcgttcctgcctccacacctttgcttgtgctgttctcacctggacacccttttacaatgtctccactcacatacttaggaaattttgtagagatttcctgcttcagctcaggtaccaatgcattcaagaagctgctttctctcaggaagctggtttctctttcctctgagcgcttcttgtattaggttggtgcaaaagtaatcacgttttttgccattgaaagttatggcaaaaaccttgtctttctaatttgggcacataattataaactgcttggtgttttcagtcctatttgtgtgtgtgtgtgtgtgtgtgtgtgtgtgtgtgtgtgtgtgtgtgtaaagactttgagttcagtgagggcacaaatggagcctcatacttccttaacaacctccattagttctatgaacttagtggatgctttgcaaattaatcccctgaagcttcagctccatgtcttccccataagatacccttatcttctacctgcatggaatgcttttattcagtgtttgggaggcagggggagagaagatgggatagtgtccattatgtgttctctacgtgtggttagatgttttctgctacttattttctactcgaggagctccatagtgttgcagttaagaacagggacgctggcactagctagtctacttcttactagctatgtgaccttggacaagtcacttaacctttctgagtccttgtttactcttctgttagtttcagcaatgatagtacttacctcagtgtgtttcctgatgcataagtaagttaatatttatagtgttcttagaacactgctgtatgtaagttgttcagtaaattgtttggcacccgcatgttttagattacaaacattttagggcaggggctgaggaccattctctcctgctctgtaccagttgctttccctcaaaaaagctttgtcccagtggtgatgcagtaatcaccccggatgtgcctgagaccccttccagcccacccagagaatacacttgcctcgtgccagcagctgtacatgatctggtagatggtgtccgatgccaggtggggccggtaaagcctgtggccctgggagaccttcagaaccacctgggagttgtcatacaagtcatagggctgcttccccaggctgaacacctcccacatcaggatccctgcaccacaccaagaggtggcccagataaaaaaggaagagagctgttatcagaaagtacagggtttgtggagtgagtttcacacaacaggtcaggaaggctatgggtcagagtgagggatccctgggaaatgagtcccagcgaaatgagcagagcttggaaggtgagcctgtgccattgcttctggagcctggggtttcttccccatgttccctgatacctgtacatctgtgcttgctggaaagtgggtgaatggcaagggcagccacagaggaacctcttggggccaagacacaattcttgcgctagtcctgtgaagtgtgagttgtcagcctcacagtgacaactacctgtgtagtgagcccctgccccaacccttatgaatgagtggttgatgtcctcacccattccgtctctcggctttggcagctgcatgcaacaatttcattgcacaatcagattacacttcagtggaaatttcaagataaaatcattgacaacattctcctttctattgctgcagtggttcctaatctcatatgggccaggcacccttttgggaatctgatgaaagccactggccgcctttgggaaagtgaatacacagcatttttgtgttcagtttcaggatgttcacagtcccccaaaactcatccaggaaattgcagtccccaggttaagaaatctcggttctggtcatggtggtcaggaggatggcatttgggatcagttagacatggcctttgaagttgacttctgtcacttacaggtgggataagtttctcaacccctctcagcctcaggttctccgtctataaaatggggtatttgcacctcatggacttgctgtgaggattaaataggataaaacacgtaaagcacttaaaagagcactgagatgtttcaagtgctctctaaatggtggcagttgtgctgtgctggccaaggtcagaaaatttcaacagtcagccagtttcaaatatttataataaagcaaccttataaattttatggctgcttttttcttttaaatcacacagtctaaaacaacaatccaaaagaatgcagtttcctcataactttctctttggaaggctacacacttatttcaaaatatctgtggggttcttctttgggaattgctatcagaggccctgattcattttgggtatcctcactgggatatattgtttaagaatgaatttagtgtttataaacagctggaaaatatctggggccaacttctatggcatacggtaaataatcaagctgggtaatactattttttggtcaaaaacaagctgtaattattaaattagggcattgattttttgctatcacttataattgatgcatgattatcaaaactaattttttttttttaagacagcctcttgctgtgtcgccaaggctggagtgcagtggcacaatctcagctcactgtaaactctgcctcccaggtttaagcgattctcgtgcctcagcctctcgagtagctggggttacaggcgcgcaccaccatgcctggctaatttttgtgtttttagtagagacgagtttttgccatgttggctgggctggtcttgaactcctggcctcaagtgatctgcccgcctcagcctctcgaagtgctgggattacaggcatgagccaccgcactcagcctcaaaactaattttgacaacaaggtacaaaagttaatcagtacaacatggcatcaagtattcaccaaaatgctatctttgtgattttttttctttttctgtgtgtatttgattatctttttttttaacaacgaacaatgccacttttacaaagggaatagttagttttaaaaattgtgtcacatagagaaataaataactaaattttccccttttcaaaggcagtcttgtaggtgacaagcagtaaatcaaattacacttatgaactaaggcatgaaataggtctcagtcacatttggattacttatcctgggtccatccgacagtgattgaaaatagccttgatattctcacaacatagaaaaaatatcataaaatgtagtcacaatttaagaattgtttcccttttctaatgacctttactaaattttaatatagatattttagtaaaataagagtcagcaaacttatcctataaagggccacatagcattttagactttttgggacatgcagtatctgtggcaactactctactctgctgttgtagcacaaaatgagccatagataatacataaatgaatgggcatgactgtgttccaataaaactttatttacaaaaaccaactgacctggtcctgggctgtggtttgccaatcccaatctaaaatataaattttaacagagaggagactacttgaaactaggcacgagtcaaagaaagtaccaagtcatgtgtggtgtttacactaagaattggtcagcgcagaagggaatggctatcccattattttcatgcagaaaattgatgtcttttcaacgtttggggctcagcaaaaactcagattcagctttcaatgactccttttaaaaaattatttgaaatcttcactggaaaaactttcatcccctttgatgtttctaaagacagggtcttaactgtgttgccccagctggacttgaactcctgggctcaaacaatccttccacctcaacctctgagtagatttatcccttttgattttatgagaaagtgctagcaggatgtaaattacaagatgtgtagaagacatcctaataccaaacccaagaaaataaatcaccttcttcaacagcttgcttctctcaggctatatgtgtaacttcgtaagtgttcaagggcccttttaaaatggacattgctactcttgtggtttcctgttgtggaagtaagcttatgaaacagggccaggtcgtatgtggccacatccttaccaaatgcccatacgtctgacttgctgctgtatttgaagtaatgaaacacctctggagctgaccacttgactggaaactttgttccgactgaactgacatactggtcatcaagaacatacctgccagggaaaacacatcaacacagaccttaagccgtggtggtacaggctgatgaaggacacggaagccacccagagtctcagtcgattgcctcagttttctggaacttttgtttcctttcaatgtgtgggcttagctggaggctgcttatggtttgtaagcatttaaaagataatatctaactgaatacagcaaaacctcaatgcaaaatgtttggtaaatatagaaaaacacaaaaaagtaaagataaaattactcagaaacccaccctaaagataacctgtaattctatcactcaaaatacatttttctttgtactttctgatcttatttcccacatatataagttgaacaaaatagggtttctgttatattgagatttgcttttatttttcctttaatggtatatcatgagcatttccccatacggttacatttctactgcagcatcatgtttagtggctgaataccattccctcttattatttcttcttatggaaggaattaaacataatttatttaaccaggtaaattgctactaatcttctgctgttatgaataatgatgaacaaaaatatcctatggagatatcaagaccaaaatacatgttgaagaatgttcactaaactctttgtttgtaataatgataaaatgaaagcaacctcaatgttcccaatgttgaggagtaattaaatatattatggcacactcacacaatagaatagcacatagccattcaaaaatgagataactcttcaccaaaataaaaaggtgctcatggtatatggttatgtgcaaaagaaagtgtgtataatgtgattccattttttgtgtgtgggaacaagtagtaaatgtgcatatttgtgtatatgattggatacacttaagaatgaggtctgaaaggatacccaccaaacatctgagcagtggttactcctagcactgaaaatgagggacagattctgtcagaataggtagctaggcagacatgagcagggcaggagagatcccccacccccaacccaggaatattaggtgaccgtcaggtaatagtcaggcagttgttaaccgtctctctaaaataataattggtcgcagctggtgccagggaacggcagtctcccaatagatagaaaatacttgaagctgatgatcaacagcttcctgatatgatctcaggagttgggcaactggggtcaaacatgcgcaataagaggcaaaataatggagtttaactggtatatgaccttcctctaagaatactcgactggtaaaagaaaaatgcctcaaatgagcatgcgcacaacctaagtaaacacactgtgcgtgcggcccctcccaagtgctggcaggctactgctcatgcagacagcccaccccaagggaagaatcaggggagaaaagatgcaaactttggaattatgccaatgtatgaaaccctaagtcaagggccaagtagggcagttggatctctcaagttgccctcttggccctcttccaagtgtgctttacttcctttccttcctgctctaaaactttttaataaactctcactcctgctcaaaaacttgcctcagtctctctctctgccttatgcccctcagatgaatcctttcctttgaggaggcaagaattgagttgctgcagacctttacggattcactgctgctaacaattctactttttactttctacactaatgctatttttcttataataagtacatattatttatagtattaataaaagagaacatcatatatttgcccacaaaaaccacaacaaaaacaaacacataatgactcttaattagagaaaatagaacaacaggagttatggcaattggaattttggatataatttacacaccaaatgttttagagatctcactgtaatttaaaatagctggctgggaagatcatccttctaaatattaacaataatattaataaactacactttcataggattacatacagtacattccatacttttctccccagaacccctcagctccccagacactaccagatacccagacactaccctggcactcctggtttggggatgctcatggcttaaaccttccttcaagctagtaggagcaaatgaagtcacaggcatctaattgatgtggccacagagcttcataaccaacagggctggtacatggcttccaggatctcttactcagagcaagatctcttttttctttggatctttttttggaaaagtctcccttttccaaagctacagagaacgtgaaagcatgttccctggatgctggcataacagccaatattcttgcttttagagtaaaggtggggatcagttcccttgacctgggagcttctccctgctttaagaacctgtcagcagattttttttttcttgctcttgagaaagctacatgtttggagggtcagtgtaggggactcagtgtatggtggttggagtgacggtagctagtacacagctaaaaggacatcttgccaatatttagggctcccagaggaattaccatagcttccacggtatgtgccacaagggtcaaaaagcatcccctaacactgaattttggagaagacattaaaaaaacagatcctcctatggcattttagggcaatgtttgctaaatattaacctggccttggtcaactcttcagatctcttttaaactgcattattcaccttcagaattacatattgagtcagcacaactttatagttatattttgtctcaatgtcagggatatctacttgccctctagactaaactgtggtcattttgagagcagggactatcctattcatatcagtatctcctgcaggactgaggtattcagtaggcactcaataaatgttgaatagaaaggttaatgtgtatgtcttcgttttctttgcaaaactatgaaaatttgctctgaggcttaaatagtattaggatacaggattaaggtaaaagaagactttaaaaaaacgatcgtaatcaaaataattgttccacatcattaagtggtggtagaatgcccatcaagagaatctcagagtctagttttatgccttactttgtgatgcagaaaatctaatgatgtgacaacaaaaattttcctagtgtgaatgctctacccatttgaacatcaggttttttttttaatgacagacaaaaagagctagcagatccttgattgagttaaaaaattataaatatttatccattctttttaaatggctgagtagtactccaaaatgcttgaggggatggataccccattctccatgatgcgattatttcacattgcatgcctgtatcaaaacatcacatgtaccccataaatatgtatacctactatgtacccacaaaaattaaaaataaaaaaatttaaaagaaagtaaaacaatccaataatgtggctgtaacatgttgtatttaaatttaggatctttcatggaatttatttagaagtttagaaagtatcagattgttgaaagaactaaaagtgacgtcagataatttctgaagaacaatcccatgggagggacttaggtttctaagcccccaattcagagcaccttctatatccccacaaagccttcaattatgatggtgttcaaatgtgaatggaaatccccctttcactggttgccctttcggaattggcttaccttgtcattccaaagtcagatactttcacacagagatctctgtccaccaagcagttacgagcagcctttaaaggaaaaacaaaatcaccaagtagtgaaagtgaccaagaatctcctgacattttcctccttttgagtagaattctcagcaaaatctaagattacctagcacaacccccaagtgctctaaagttgtcttttttttcctttgcaattttcaatttttcaaaataaaaatgttctcccatgagtactttcaaccaagcatccatccttgtcaaacaacatgaaaaggtattacactttaaacacagaaccttagcttatcaaggaaagatgagaaatttctagaatctccctttccaccaccagataccgtgcattcacagatgctttgaaggcataaacgctgcacctgtgacttagggaacacactggcctagtgtgaatgcaggtctactgttgagcgcaagaaaatctctaaagataaatcccagtgaataccaaacacagcattccccactctcattctgatccaaagacacacaaattctatttccttttttttttttcatgttcagttattgtatcaaatggctgctttagcactaagcccttcctgaaatgaaggtcaatttgggtttaatggttttgagagacactaaattggcagaagtctttcctttccatctgttctccttcttgccacaacacagctatgtagaccttaacagcttattttattctcactgttctttccatacttcaaatatttttacattgatttttgtaaaaatgtcatttgcttaaacaagtcacaagtgagcaaagtagaagtgatggtgcccttcttgccttccctaccagtgaccccatctccagaagtgggagccgtggactgctttcttttctttgaaattccaatggctttgcttaccaagtcccggtgtatgaattggtgactctccaagaaggccatgccttcacagacatcgtagcacatttctaagagctgggaaggttcaagtccttttccgtgactcctcaggtaattcagcaagcagccattgcttatatattcagtcactatgtatatggggtattcctttgaacacactccatagaatttaaccagcttgggatggctgagtttcctggaaataacagattttcaaagttcaatgctccacactccattttctacatgtactgaaaaagagttgctctcagatttctgtttgttttaattcccacccccaccctctttcacttgaacagagaaagagagggaaaagtttaggatcttatatttcaagcacaattatctttggaggcaaaaaaaatttctgcctcatatcagacattggtgacttcagcagcaatttagcagtttgtgaatgctgtattcaactttcctacagagaatcagggtagcaatggtctgttgcactggaaaccctgttgagtctcatggcatcctgggaacgtccatgacttatttagaggaagccacaccctaagaacaatcatttctcttttattaactttcacatctaaatatttcattctaaaaaagtatattagaatacgaagaatgtaggctaaatgaagtcagatgaggtttgattttttttcttttttaaaaaattattttacattattgttttcattatttttgaataacaaatttataaaatactaaccatatgatgtagcacttctggtaggtcatgatggcatggacactcagatttttttttaatgagataattttcattttattatggcagatggttttaccgtacctcttcccagcaccctttttaaaatgataaagttaagtagaagaaaatggagcgtaatgtagctatgggctattatttagtaatcagatgttccagatggaagtgtttaatcttgcgaattgaagagtcctttagtgtttcttattaatttaagtaatatcaatcttaaattttgtactagcctagagcttaaaagaaatcaagcaaaccttcttgtaaagtgtgattcttctaccttaattacctttctcatgatatcttttctcacctatctttttttttttttttccatttgtgagatgcataggttaagctaaatgtctagcggagtctctagcagtgtgctgccatatcacctttacttctccagacaggctcccgaactgctgctttactctctttactgtcttcacggttaagcataaagccttttgaaagtacttagcaactccaattactctttcttttctgttctacattgtatttcagtgaatttggacacatttttggattgacatgtactttactcttagattctgagctgactctgagtgatccattgtacaaacaaacagtgcagtagtgtagttgttaatgagtttggaggtattagcctttttggggagattagtcttttgtccataatatgaattacaaataattttgctcagtttatcatttgtcttttagtttgcatagggtttttattttgttttgttgccacgcagaagttgatttttaaattttatttttttttttattatactttgagttctgggatacatgtgcagaacgtgcaggtttgttacatatatatacatgtgccatggtggtttgctgcacccgtcatcttcattaggtatttctcctaatgctatctttcccctatcccccaccccccaacaagccccagtgtgtgatgttcccctccctgtgtccatgtgttctcattgttcaacttccacttatgagtgagaacatgcggtgttcggttttctgttcctgtgttagtttgctgagaatgatggtttccagcttcatccatgtccttgcaaaggacatgaactcattcttttttatggctgcatagtattccatggtgtatatgtgccacattttctttatccagtctaacattgatgggcatttgagttggttccaagtctttgctattgtgaatagtgctgcaatacacatatgtgtgcatgtgtctttatagtagaatgatttataatcctttgaatatatacccagtactgggattgctgggtcaaatgatatttctggttctagatccttgaggaatcaccacactgtcttccacagtggttgaaataatttacactcccaccaacagtgtaaaagcgttcctatttctctccacatcctctccagcatctgttgtttcctggctttttaatgattgccattctaaatggcgtgaaatggtatctcactgtcgttttgatttgcatttctctaatgacaagtgatgatgagctttttttcatatgtttgttgtccgcataaatgtcttcttttgagaagggacactcagtttttacaagctcattaacttgttcttatatgattaatctaattttaacaatttacaggattattctgtaatgttcaaatgtgtaaagaaatctaaatttaacatgtgaagttttaaatattttaataagaatttattaaatttttagttattttcttccttcttagtttaactatagggacaatgcttatcatgtcactcaagttgatgagcttgggagtattcatcttttagcagttttggagacctgcatggtactcaacactgctcccttcagagggaaggcttgtgatggcacaaaggtccatcacccaataaagaaatattctttgaacaaattcagtcactgcccaggtggctcaaggtgagggcgccatattaactgatattccccacatgataggcttcagcttgacccctacatttgggattagactttttcccattgtggcacagtttgacgtttttctttatgattaggtgatgtttttaaatgacttcttgtgatttttttatggataccagcttggtaatggcaaaaatatgttttaaagttacaccaagacacagaggttacatcatgagcctgtaaccatgagaaaaatctggcagaaagtttctgccagattttcctgtcctacaagaggagaatatcaggggcccccttcccagttttatgacagtccaatcactgaaggcagaaccagttacataattgcaggcaccaatgcaaaatgaaaatgcaggaatccttgtttaaaaatcacgaagtattacttcaagatgacaacagcagagcattaaaccaagcacagggcccttctaggtactgtgcccttctaggtaccttctaggtactaggtacttctaggcccttgcaactgtgcagatctcatatgcccatgaagccagctagggccaaaggcctatattcacatagcatctctgcttccaaataattttgcaattatggtaaaagtgattgttctgactttcctaattcctttggtcttcataaaagaataactaaagaaatggaccctagaatgtctgcctgggtttgtatctctgcttttcgacttttactagcttgccgactctggacaagttattcaacatttctgtgcttcggtttcctgtaaatctaagataacgatggtacaaccctcataggtttcttatggggattaagtgagttaatatttgtaaagtgcttagagaaatgcctgtaaacactgtacagtataactgtttaagtaaaaatttaaaaagtgctctcaatctctagcaggctaggaatttttttatttcaactatttttgaccagagacctactataacatcatttttcataccatactgattgattctctaaaagtagagtgaccaactgtcctggtttgcctggggctgggaggtttcctaggacacaggactttcagtgctaaaacctcccagacctcagcaaacctgaatggtggtcactctacaaagtctgatttactatgttgacaagaagagggagaattatatacaactgagagagggagagagagggagagagagagagaaggagagagaggaagagggagagagagggagagagaaagagagagagggggaagagagagagagagagaagacacagagggaaagagaacacatatgtattgccaataattgaaggctgttgtgctgggagctgagacacattaggaagagtattagactgtattgaaggtaccaagaaggaaaaggatattgttggtagagcattaattttcagtactgcattagttctgggtggtagccccactctgttcttcatcccaccatgcacctccccacagggcaaggaggccacagggccaaggagacatgcctactccgaccctatatgtccctttctctgccacccttagagcaccaggactctgaaatcacccaaccatatagtcctgaggcctctttcaggacctgtgctgactcttccttaaggagtctgtccgcttaagggtaaagcacactacccctagccaagatcattagccactagataattgtttgcgggagacaaagagggagcttgtgtgtactgaatgctgtgtctgtacataactactgaggccccttatagtgcaggacggagttgggtatgaggggagaagcaggaggggtggagtttggaagaccacaggccagaggctggtttctttgtagagtcttgggaattctcaatttgaatatggcctttcaggttgttatgaaggtatatttgtcaaagtaggagcataagactgatttgatttaatagtctgttagttcaatttataacttctaaatatttatgtctatgatacagtatgtggacttttgctctaggccttgcaaagttaacagcaggcctattagtgctaatgccctcccatgatgagggctaaacagccattccttgggaaaaacttacatcatagtctgggcctcctgaaagaattcatcttctgacatggagccctccttgatcatcttaacagcaacatcatactgccccttccacttgcccagctggaccactccaaactggccacttcccagctccttcaacaaggtaatctcttctcttttcagttcccagattcctaacaaggcaagacggacattctttaggacgagcatagaaagctttggggctaacagcttcccaagtgatttcttctccaatgggagtttcacagtgacaatataaacaagactttttattttaagttgataagttaaaaaaaaaaacccttagtgatgtggtcttgtggctacgtgataatggcaccagggtaacactattcttgaagtagctacagttatttaacaagaaggctcagagtaaaaacaatttgccaatcagaggcatcttggagaggcttagcatcaaaatgtactcgatgtccttagattggtaaaacattgtcagatttttttaataaaaaaattaaactgtgatcgcagtggatgtgatcgtcctaacacacaaggttgaatactggctctgctcagtgggcacaaaatccaggaaaaagataggacagacaatggtgtgctggaggctaataatggggtctgataagaaaacaaattgatgatcttcttggtgattctagaagcttacagatttaggttgagaggacagattttttttttttttaaagaaaatccttcctgatacaatctgaacagaggcctccttcctgtctttcctcctttttttttttttttgccagtaaattaaccatatatttaaataaaaaatgatggaaaatggagtttaagaacccaagtatgtatccataccatttcccagggacacagagtcggggaccttgttggcctttgttgacacagggtggcggagccgtgtgatcatgcctgcaatggaatcagcatcatatcatcacatcattatatcatatcccatcacattatgtaacattggttcacaactagtctctctccaaagtggctgaaaaagcaaagagttcagaatgttgtaatgcagagagagaatccattcagttgtagttgtgcttaacttgactaattagcttaatgcaaagcgaaaattaaatgaataaaacactgtatgtgatttttaaaaatcccatagaaattaattgcatgatgtaatcttttaaaattctcccccttcttttttgctatctctttacaatcttatgcttaaaatatgtatacacacagcacatacatttgacaaaaaattcaaacaatacagaagggtaaaaataataagccatctgaatttttaaaaactgaacctattactaagtttcattaagtggtcaagccattttaattgtttatatttagttatggtaaaactgggttaaagtaattataaaatttaatagtggcttgaaagaacccattaaaatatatgtatatatgttttttgctttaaatgtggaatgctctccaagtcaagggacctatctatcacacatgtacttttcccctccttcctttttctatatatttgtctggtttttatatgagtggtctccaaacttttaaaatcctttacctttaatcaactaaattaattaattaaaatgtaaaaattagtacatttctataaaatatatatttggtgtgaattataaaaataaactcacaaacagaaaatgtaagagaatgagataaaaatccaaatataaataaacatcttaatatttccttctgatatcctgtgaagtttcctgtgcatctctttgtgcaataccttttacaaaaggtacttctgaggtactacaggttaaaataaacaattgaggaaattggggcaaaggaaaaacatcagtaagagccaaaatgaaagccagaagtgaggtcaattcacctacatgcatgcaatcaagtcatataaagtaacacaaattaagtgctgagcttatggcttatttttactcactgttggaaattcatccagccactgctcttaagtggcactattctagaaaacagagggtatttctagatgtatttccaccagttgattaggcaaaaatttagaagactaatagcacccagtgttggtgaggatgaaagaaaatgttgttagtaggattgtacatttctgaaaagcagttggcaatctgtacattttaagtattcttagcctttgtctcagaaattccatttctagggatttggctatagaacccctcatatagatggacagagctatgcatgcagaattgtgcactgaaacatccattataatgtgaaaatactggacatgatccaactgctaatcaacagagggctaggtgaataaattatggggtaactatgatagaattctacacagctgttaaatagaccaaagtaagctctttgtgtttgcgtggaaagacttctaagacatgttgagtaaagaaagcaagctgagcaatatgtgtagaatgatctcatctctgtaaaaatgtaatgtatatacataatatataatttctatttatatatgtaatgattagtaagtgctagtggcagcatgcaaaaaaccctgtcataatagacaactggtccatgatccaacgatatgtatatatttctataatattttaaatttataatcctcttgcttctatacacagaaaaaccctacgaatatttttataaagagaatttctctttctaagtcacccagtttactcaatttcattctttctggcattaaggagagaatcatgttatattcctgggaatgtaagctaaaaattatgataggcaaaagacttgagaggattttttcctactttcatggttccctatataattaagaatattaggaggatcagtagtatccaggaaaattgctgttttacaacaagggcccataaaagaaaaactaaaataagttacctgctgaattgtgttgatgataatgaataagctttggaatggaatcaaaacagtagttttctgccaggtataatttgttctcagcatttgtatgcacgtggtaatgtttgacagttccttttttatcactagtaacagaaagaatgttagaacatttaaaataaacaataaagcttgattaagcaagggttaaataattattttaggtgcatttgcaaattttggagagggaagtcagggaggttttctcaggcttggcatctggtgtttttctcagtcttgaaatctggtgtaactataaaagtaattatggaagagagttcttctctgaactcaatgtatcttttggtgcacatatagatttcatgtacagtaaaattccaactttccaggcttctcctcccttagtaagtgtataaaactggaatctttctttgatgatcgagagaggcttgtagttcttcaggatcattattctgaacataaattatcttataggataataatggaggtggaaatgagagaggtaaaggtgattaggctgttgggtatcctcaccccaaaacatgggaggaggctcttttggggcatgcagaatgaagtaggtgaggatgtcctttaattcctcatgtgaacttgtaccttctccacataaaacacttttttcttttcctctgcttttatacaataatgctgaattgatgcacgttgatttaagctaatcaggaataatacacctgtcttagattgtagacaatgacatagtggtcaagaaccaggctttgagtttagttctatcacttgctagctacctatatggttatgcctggcatataagaaacagttaataaattgttgctgtatagcaaactaacacaggaacagaaaaccagataccacatatttttacttgtaagtgggagataaatgatgacaacacatggacacatagaggaaaatagtagacactgaggcttacttgagggtggagggtgggaggagggagaagagcagaaaaaataactattgaacactaggcttagtagttgggtgatgaaataatctctacaagaaagccacatgacaagagtttacctatataacaaacctgtcacatgtaccaatcaacttaaaataaaagctaaaaaattaaaaaattgttgctgtaacatcaaccgagtaaaaattgcaagtacaaaaggagaaatggttcttctctttgctgcaaatgtttaatcagcttttggtgtcagcatacaattcactcaaggccattgatgaggtttcataaaagagggcttctgtattcatttgattctttatctatttgctgatttatctacctggctaccttaaaaaatgaatctgtagtcaagaaactcaactacaaggacatctgataccaggtttggctgaaactgcagggccagaaggccacaaaacttttgatagtcaacaaagtcttctcctgattcttactgggttggctttagccagcatccaggtgttaccatgtgcaaaataatatatgagatacactggggacatggattctggcattggaaagtgctgtgggacaattcaggtacatcctattatctcaagtgtaggtaggcactctgtaaaaaatttcaaaataaaataacgtttttattacttaattattataaatatacatatatgcactgtggggtagggcacatggggaaggcaccaaggcagacgatcattcaatctagctgctaaaaaagagttgcacacatagaaatttaaaagaagatccgaatcaatatttgaccagattctcattccaaaacactgggggttcagtgtaaacacagaggtgtagataaagaaacatgacttggctattggaagcccagcttcaaattagtcacttagtagctccaggtaggtctttataagctctttttgaacctcaatcctctcatctgcaatatggtataaataaaaacagctttcccatttgctttggaggacgattagaaggaaaaaaaaagagctaactaccaataagaaagtggcttgtaaaactctggagaaggattttgatgatgatttgcatgagatgactgtggggtgtcttttatgcccaagtgttagggtacagggagtggagcaggtagaactacccttccccagcaacaacaacaacaataatagtaagtactattagtttattaagtgcttactatgtgtcaggcactgttctggatataatatattaattaactcaccccactcatagaacgtaacaatcattatgttcccctttcaaatgaagaagttgaggcacagaggctgagaaacttgctcaatgtcacaaagccctattgaaatagtagagccaggatttggatctagaagtttggcttcaaacaagaacttttagtcacacaaagaggacagagccctggaaggaagctgtgtagagaacacagcagcactgagtcaaccttctgctaactctgcacccagtggaagggcttactaagaactggacactggagtaggcatctttgctcagcaggtggctagggctgagagtatagacgctccattgctattttttttctttccaggcttagacctgaatgaaagggaatcacccgagagtctcttaataaacatttactgaggcatgctgtgctctgggcactctgttactgcctggagtggggcaggagaggattagccagagaatggcctcttgaaagtagtggggcaaggatggtggggcagtggggtgagagtggtggaggcaactacaaatggtcacattcaatagaaatactttgatttaaaattccagagtttaaagatgatgtcttatcccaaaatgtgattgtgaccagcatattcaaagaatatacgcagaaagagaaaagaaattaatccgtatcatacttacttcacagccttactaaataaggacactgtgtacattcccacttggctcgaatttctaaccataaatgctccttcttttccctgaaaaggaaggaaaatatagaaggaatagaaaatattttcatcattcttgaaaatgattttgcatggtaggtagttggaaaacaaatcttttagattgactttatatatcacaaaaggcacaggtttctcactcaatctctttatagatctgctctatgtgtatgtgtatgtgtatgaactcttcctctccaattaatatttaaccctatcacttacttttctcggtgttgtgcttatgcaagcctaatgttttcatctttaagaaatacaatttagaaccgtgtatcaccgtatcatgcctttggaaggttttagaaaaccaaagatcaagaataaatcagttgataaagttctatactcaagttgtctttctcaatgatttgtgctaattctaagcagatcaaagttcagaaagagcagattataaactatgaatgtgacaaaacgccaaatgaagtcattacattctcactagcacccctcaaagaaataagtgtaatgtttagtgtttttatcaggttaaactatatcaaagtgtcatttttagaaatcaaaagagtttgctactggcattccatatcaattagtaataaagtcagataaacatgttctgtatcacatctgctatactctggtctaggtgctaacgagcagtgacagagctgaggttaatggctacattgacggcctccgataaatcatctctcttattcatgctgtgagcagtcccctcccatactgactctgggctgagccatgtgacttgctatgaccaataggaccttagcaaacacaacaaaagcagagactcgaaaagtgcttgcactgtggggctcgtcctcttcaactactgccttgagatcaccatgctgtgaggaggcccaagtgaggcacatggagagactgcttggggaagaactgtggaactccagttgactgcctcagctaaacccaagcccccagtcaatccacgacccaaatgcagccacaggagtgcccctaagcaaaacttgcctaagaactgctggcccagctgactcacagaatcatgagatatcataaatcccactgtttcaagccacccaatttaggagtggtttattatgcagcaatacataatggacacagaagtagtggctagacatgaagagtcgggcaacccacagactggacaagcaacactacaatgacttgactatataattagcaggaaggtttctatgagaaatgcatcaacctctggaccccaaaatgcaaagagattctgtgaggctgttttaaagacaagactatttaccttttgtctgagtaactgttcagattgtgatctggagatgttaccagcaaaccagctgcaaaaggaaaaaaattatatgtgagaatcaataagttttagattgtatagcttgacactcaactttctaatctgtaaaatgaggcattggcttacctcaggggtttccaagcctaactgctccagagaatgatttgaggagcttttcagaaatacaaatgcttaagtccccactactaaattctgatgttggggtggagtctgcacatctaatttttaaatgggctggcatttaggaatctttggttttcttctaggtgacttctaagttttcctacattttaccattttgagtaagggctagagattagacctatgaagaatattccaagaggaaagtcagggctgccaaggatgaaagctcttgtcttccaatcatataatttccttgtaataatctgattataacatctattttcacatcaaatgcttttactttatatgtggagctcaacaggccaagagctgtttctcatgtgagggcaataatgtctgtggcctttgggctttagacagaatgtcctacttaggaaatgagtaaaagcagggcatgaggtgggatgtagcatgggtttgggtgagtgggaggacccactggctagaagataacatctgcagaggaaataagtacaagaggggggtgagggtagaagggaggagttataggagaaattcaatgtagaaggtatctcagaacttggcaggaatggatgggaagtgattctttgtataatctctgtatgagttttgggaagggatgtcagctggtggacagctcaataaaatagaaagagccttaggtcacccagcttggaggtctgggttctaggcataacttccctatgtgtaattgtatgactttgaacaagccacttggccttgtttgaccttggacttccctacctgtaaaactagggagagggttgatcttgataagtttgaaagtctcttttaactctaccattatggatactgtataactaacacttgaaattttaactgatcgtttttcttattagtcaacattttaaggtaatactagctaatcaaaaggcccttcaggggctgtgatacaggatatatacctacaggggaatagaaggaagaaaatcaagaatcaggagagaggcctggagaggcaaggtgatataacctatgtaccctatacattatacctcccacacacatacaaacgttgcctgtgtcataattttgctgaggcctgcctcttattgttttaaataatatttcagtattcaatgacagagcagcagagtcctaggcctaaggtttttttttgtttgttttgttttttgacagggtctcactctgtcacataggctggagtgcagtggtatgatctcggctcactacagcctcgacctcccgggctcaagtgatcctcccacctcagcctcccaagtagttgggactacaggaatgggcaaccacacctggctaatttttgtatttttgtagagatgaggttttgtcatgttgccccggctggtctcaaacttctgagctcaagagatctgcctagctcagcctctgaaagtgctgggattataggcatgtgccactgtacctggccaggcctagtcttaacacacgattttctatctaaatctcagggcttagtgcttctgtctttaatggaattgactggaattgctcatgaagttatggccataaaacaaaaactcccatgacactgatagaataggaagctgggagcattaagcacaatgtgaggtgactgagtcccagatcctatagccctataatcccgagctccttttggtatctttttgattttgaagaggacaagtcttccctgataatttcctcaatgtctgtctctcacacaccacttgcattcagaaaagatgggaatagagactgtttaaatggtcatatctgggcttacccaatcagggaaagacttctaatttatttaaatgctaaagagtacaatccacaattagaggttcactcccattggtttaagtctttgtgtttcattaaagtgcaattatctttggaaatgaccatgcgttggtcattctcagtcttttatgtcttagtatgaattattccatcaatgctttcctgagaatagatgttagggaagaaagtaaagccgaaagagagctagaaggtgataaagagaccatgccacaaagatgtggagggtacagcacatcttccccaatgcaacaggtatggctaggaccaggctgggttagagatgagaggaggagtgaaaacagtagcatgtactttgtaaaagctctcgtttcacatgtaatcaataaatcaataatacaacacttgtacaaaaacagaactggaaatatgcataatcctagtgttcaagtgaattctgtgccatttggtatcaagtgtctgagaaatgcaatatgctctattattcccacctcttcttaacactaatctttacatctaggagacaattatttgtccctatttgtagaatgatagacccttagattttggaaagaaccttaatagccacttaggccaaccttctcagtatagttggggtgaccacacgttcttatttgcccagaagtccagtttatgtcaaatgtccaggtgtaatttttattttaatttttgtgagtacatagcaggtatatatatatgtgtgtgtgtgtgtgtgtatatatatatatatatatatatatatatatatatatttgtgtgtgtgtgtgtgtgtgtttgtgttgttttttttttttttttttttttttgagacagagtcttgctctgtcgcccaggctggagtgcagtggcgccatctcggctcaccatagcaggtatatatatttatggggtacatgaaatattttgatacagacatgcaatgcataataatcacatcatggattatggggtatccatcccttcaagcatttatcctttgagttacaaacaattaattatactctttaagttattttaaaatgtataattaagttattattaacgggtataatttttaatagtgcactttttctctttctaaagtatcccaattttgatgttaaatgaaaaggttggctgggtgaggtggctcacgcctgtaatcccagcactttgggaggccgaggcgggtggatcacctgaggtcaggagttcaagaccagcctggcaaacatggcgaaaccctgtctctactaaaaatacaaaaattagctgggcatggtggcaggcgcctgtaatcttagctactcgggaggctgaggtaggagaattgcttgaacctaggaggtggaggttgcaatgagccgagatcatgccactgcactccagcctgggcaacaagagcaaaactctgtctcaaaaaaaaaaaaaaaaaatgacaaggtcatcctaagaatagagatctgtcgacataaatattgccatgttcaaggtctcatttgtgctaccctgtaaaattactgcatcattaataacttctccaaaaatattactcaatgttaataagagctttcaaacatcaccttaattagaggtctccaggaaagaaataatgagcagaaacattatatgctacatctacaaaggtaaagtgaaatgaaggtgacgctggacatacgataaatccaataagtatttgcatccaatttagataatccagttttggggaaaatattttcgttggtaacaacccagctcattgtcacctgtcatctagataaatgacccagaacattacgtgagtttacccatgtaaatacagcctcctgagacggtctcagccatctgttttaatgtctttgaggaagagagtagaagtatctatgtctaattttagaagaacccagaagactttcaatactcactcataatcatccaggttttcctcttcttcagatgaacttgactcagggaattcccttaagaagagttgagaaaaacgtgagtgcagaaaagcaccaagaaataatccaagtgcgtagccatgcaatatcaaatcccaccctgattttatccacctaaataacatttagattgttaggtttctccttctgcacctggcaaagtatcaacacagcaacagtaaagagagagtcactcaaaagttgggcttatgccaagttttaatgaaagaatggttctttgatacaggagatccccaggaggcccccagaagaacttatatttctttgactttttccaaagagtgggctcacgaaatagatgaagactccttcagagatggctgtagtatcaagttaaaaaaaaattgggttgtgtatatagctattcttatggaagagttattcaactctaggttgcttttcttcctgctagagatgcctgggtcaggtatactaatgttggtgctactactggtgtatgtcatggatgctggctaggaatagtcagttcaagaaaaactagaacccaaaagccatccattcagggttcagagaggcaaactacatagacttttttggtttatatttaccttaagtagaaagcctagctgcaaaattattgcaagtttggttagttttcctgaaaatggaagcatattctggatgtaggagatatctgaatttgatttaccatgaaatctttgaggtggtgtgattcacatttctttctttttggttactgcttgcaacatcttcactgctgctgctactgcaaaaaagagacattttataagtttataaacatatgtctacttataaattttaaatgacaaaagtaattttgttcagatatgttttagctttatttgacattttgacccacacatttcacttgtttgggaaatttaagctaatgagttactttcacattttggttctttcaggcattccaataaaagcagtagtctggcaacacttctaaacaaaagaaaattgttaataatggctcataattatcttaaacaatggaaaatctagattaataaatcatcttgactggatttgccctgattttcttatatatatgcaagcattctgtcacttatatattttaaaataacttattttcctcttagctattgtgactacaattaaaattcttgaaaatgctgatctttttactccttctggacacagatggataaggaggattttcatcatcctctgccatagatcattttatctcttttatttggggtattaagcagagcatgaagaagtgcctacctctcaaagtctaaaggctgttttatctggaatgtggaacctatttcctgtttgccttcatagtccagacttttttcaaaaccacagttcatagaaacaagtttccttagcaaggaaatgctcccctggctctatttcaaggaatgcctagataaagcccttaaactctaaaatgcctctctctccaccttcccaaagagccttgcccactagaatagcttttatttagccttgttgtttctaaaaacacttagagaatgtccatcaatagtgcatcacttaggtttacctagtatctatacaactggtacttacagacattaaaaataatcaggcaatttgcacaacaatgtaaatgtacttaacactactgaactgtatgctcaaaaataattaagatggtaaactttatgttatgtatatttcaccaaaattaaatgaaaatgaaattttaaaagtcaggtgtatcctcatgtattcaaataggaaaaatgctgaagatgtcaagtgaaaagaacaaatgacaaagactgtatcgtgagaacttacttgcttttgtatgcacatggccacatgtgcatagaaaacattttaggggaatgtaaatggaatattctgtaatatttgaatgatttaatgggggcatagattatcttactaaacagaaagtagattaaaaagaagctcagggaagattgtaagaatgacagaaagttgataaaaagtattatcccatatatgagataagaatcatggctgcactttaaaaagtcaagctccatgaagaccttttagcaattattataatttacttgcagatgcaatgctgctcatgctaagaggtgatcaatcaatcctttgttaaaataactagcttcctgaagttctagggaaaatccacagttgggactaaacctaaggaggtgcaaatctaaaggagtgtcaaaaactcaataatggcagcaaccccatgtgaatcgacagttcctcctggttatttgaaaacagggcagtcagcttcacctcacacctctctcaatgaatcactggagtttctgtcaggaaggggacctggaacacaattaacatgactttcactctccttgcaactgggtttactgatcctctccttccacaagaatggtgggctgcaggcgcacaaatgtaaccccataccttgggcaccaggtatagattaatacactaggattctagctccactttggaatgaaagggaccatggcttattatactgaaaaaaaatcaagaagaacacacttacattatttcaaattccatatggccctgtattattgaaactttcagtaattggctttaggcaaaaatgatgaagatggaaagaacaaaggaataaaaggacatgggaaaaataactaagaaaagagatacatggtgagtcagagagatgctctgtatccattgtggggaaaggcagacatgacttccctgcagggatgctgataactgagtgagaagatggcagagagtagacaacatgataggaaggaggtctgagacaggaaataaggaaggggagtcacttgaccctttccatatggtcatcttgcaattcaattcaacatttattgggcatcaagtgtagggcaaattcagtgccataccaagagatacatacatacacaatttacatgttgccttcaaggaattttcaattctggctttagatagaatctttaggctaaaaactgtgagaagactaatcaggtcaggagaggtgaggttgtacattccaagaaggcaagccatggccttgcttgaaatgctatttgccatttggctagtgatactgaagcacattacaaaagtaagcccatcctaagctctttcatataaaggatagatttttaaagattctgtattctggaatgtggagatatttggggggagaaggaggagaaagggaagggaatacaatgatcaggcattactgagatcattagattggataatctaaatggaaaatctatgcttctcagccccagtggggccactgcttccccagacttctggctcaagcactgaacattttctgagaggacagcctaagacctggatcaacagctttgaggctgaggtccagggcagccggggtagtgtttactctacatgctgggcagcccgtctgaaagctggggattaccttttcagttttcttacttgccaccagtcagggaagtcttcccttggaatatactgcatgttgaagtttggctgggagccatagattttctttgagttgctgtcatattgcgctagactggtacttgaagatggtggctgggagccatagtttttctttgattcgttgtcatattgggctagagtggtacttgaagatggtgcatccattttgagaacaggaactgcccgaggtatcttcagctttggaggggaagaaattttaatacatcacagtgaggcacagattcaggtaccgggaactgacaattaagtatatcagctcttgaaagagagagaaaggaaggagagaagggagggaggaagggagggaggaagggaggaaggaaagaggcaaggaaagaagaaaggtatgagcaaggatatggagcaactggaactcatattttgctggttggagtatgaagtggtacaacaacattggaaatttgtttgcagaatctagtaaatctggatgtgcatctggtcataccctatgactcataaattttacgtatatatacacaacagggaaaccaaaagacctgtaaaagaatgttcatagcagaattattttaattagataaaaactagaaactgcctacatgcccatcaagaggataaaataataaacatgttgtggttgttgaaggctgaaaaatggtctcccgaatatgttcatgtcctaattcccagaacctgtgaataccttacgtggtaaaagagactttgcagattgtaattaaggatttcgagatggggcaactattctggattttgtgggggtgggcctgatgtaatcaaaatggcctttataaaagggagataaggtggtcagagattagagagctgtgaggatggaagcagaggttagagagtagaggtcctgcaaccttctggttctgcagacaggggaaggagccaggaaccaataaatgtaagtaccccttaggaactggaaaatgcaaggaaacagaatcttccctaaagcttccagaggaaacacagctgtgcagacccattttagacatatgatcttccaaactataagagaataaatgtatgttgctttaagccactaaatttgtgtagtaatttgttagaacagtaataagaaaccaaaacagttgtatattcacacaactgaattctatacaatgatgacggtaaatataatcacatactaaaaaaataagcgaatctcacaaattgaagaaaagaagacacacaataaatgactacacactttataattccagtcatataaaagtcaaaatcaggcaaacaatctatgatgttataaatcaggttagcaactacttttggagggtagtagtgactgaaagggaacatcagggtgtccctggggttctgtgatgttgtgtttctttatctgagggtggattatacagaattgttcagtttgtgaaaattcatcaagctgaacacttaggatttgtgtgcttttctatgttaatgttatatttcagttaaaaattcacaatatgtgtgtgtgtgtctgtgtgtgcgtgtgtgacagaaagagatgcatttaatatttttttcatgctatattttagtttttcagaaatttcatttacttggagttaaggctttgacattctggaaattttaattatgcaggacagtgttatttatgagctgtgaagtgggttatgtccaaagaaaaatgcactaggaagttccttaaatggaaatcatttaaggaaatctctttgaacagaacaactagagagctgatttttatttcctccattcagaaaataagccctctactgaatactctgataaaaaacaaaacaaagaaaaacatactttacatgataccctctgatccctggttcatttgggaagcttataatcaaaacaggcttatgaacccttagccactgatttgcctagctgataattttcaatatattggccaaaaagttccctgctggagactagatgcaatggtttctggctcttaattatcttctgctcttagacttgtcaaaacaaactttattggtagttcatcctttttaaagtgcagaattcagtgacttttgtaaatttatcaacttgtataactatcaccacagcccagtcttcccatcactccaataagatccttcatacctatttgcagtcaattctcatccctactctggtcccaggcaaccactgatcaccctttgtttctatagatttgcctaatctgggcgtttctcctaggtgttttagagggccatacttttcaagcttcaggactgaaacatattttcaaacccaaacagcaatgttgctaataacaaagtaaaactcctaacgattataattgtttcctagggctaccatgacaaattgctacaagtttggtggattagaaccacaaaaatttattgtttcacagttctagaggctagaagtcaaaatcaacatttcagcattgccatacttattctgaagcctctaggataaaatctttccttgcctcttccagcttctggtggttccaagttgctatggtttggatattcgacccctccaaattttatgttgatgtgtgatctccagtgttggaggtagagcttaatgggaggtatttgggtcataggggcgaatccctcattaatggcctagtgccattctcatggtagtgaatgagttctcactctatgatttcccatgagagctggttgttgaaaagagcctgctatctcccttccctctctcttgtttcttctctctcaccatacaatttctgcacacactagctccccttcccttctgccatgagtggaagcagcctaaggccctcatcagatgccgatgccagtgctatgctacttgtacagcctgcagaacggtgagctaaataaacctcttttctttatcaattacccagcctcaggtattccttagtagagcacaaatggactaagaaacaggtgttccttggcctgtggtaacataactccaatctcttcctaggtcttcacatggccttctcctctgtgtatctctctgtgtcctctcatcttactagtcattggatttatggcccactctcatctttcgtgtgatttcatcttgatctttaactaattacatctgcaaagatcctagttccaataaggtcacattctgaggttctacatggatatgaaatttgggggacattattcagtctactacaataatggattatttttgtctttcatgttgattcatgaaaattatgtggcttttacttagaaagagagaagtgtgtgcgaatgtgtgtagctgaaaataagtacccaagacaccatagatccaatagatgggcggagatgtccccttgctttcaggtgaacttccagagaaccaatgggaagccattttttgtgtatcctggcatttgaggaggctcacacctgcctgggaaccctaaaaacagaaggctgtggctatgaacccaagagagtagagcaaaaagacagcagtagcctgtacttcgagagagagctacatacagcttaattaaccagagcaatcaaggttttgtttgttttcttataggccaactccactcttgggaaaaagttggctgacagccatcttgaaagattgagggctgaaaatccaagaactgaggatcaagatctctcccctgtcataaaactacatatggatctgcccttcagtaggaaattcctaaaagtctcccatgagataaagaatcagtgctggaaaactcactccgataccaccaccaccaaatcatgatagaaacagctatgtgtgtctttttttaattagacctcatcttccttggaactaactctgaaagggccatgaatctcagcccccccaaaatccctccccaaaagcatgctgccaggtgatgcaggcccaagctaggtgacagatgtttaacttggaatgatgtttgcagtcatgtgataataacattggatggaacaattcagaggctgttcttatgattacaagtaatggggacatttttatcatttgagaatgactgcaaaactatggaatttggcaaagactttatttggaagcagggaagaaagcccactgaatagctttgaagggataatggagggaaagaattatgttgttttctgcttttgtcctatagagtttcatttcaacaccaggatacttccacaaagcagtcttggccatgttgatggtaaggaaagaatgacagctaataacagctgcctgttatgtgtgatgccatcttaaggacatctcccgcatgcacccattttttctttttttttttttggtgactatttatgggcttactggctaggaaaagacacaacaatgaaatgcaaaaatatgagatattacaatggttctatatttgaatccgctattcaataccacccatagctattatctagagctcatggtttttctctctttgctgagaagtctaaatgaattccacattttctagtcttgaatctggcaactcttggtttatttaatatatcctgaccatgaaaaacatttttaaaatgttgacttaccactctgtctgggaaggtgggaactctgtgtttctcttcattgactgcagtatgcagattagcatctgaacattaaaacaaagtaatatcagaaggaactttaaacaaaacaaagagaaacactcaatgttatgaattaaaagaatgaaccagcttgtgtgagcaatactccagattaagtgaatccagacacaacatctggagtgattctgtagctttcctatataaggcccaactcctatgtaggccatgtatcctggaaagaggatggatcaaggcatcttggacaaaaagaaataaagacacctaactttttctcttttagttgttatgcccatggacttcccagtagctcagcagaagaagccaagggaataaattatacttggacagatagacagtattacagctgctcaggagctgcaacacctctacactctgtagttttcctcagaggcacagcacctgtggcacccagaacacatggtgagtgtcatccagacagagcctaggaaggcaatcccaaggcagtgctctctctggcctttttcctgctccttccagaaagttagggcattgtcaaagctggaatctgacctgctaggtaatgataatagaccaaatagaccatagacaaaaccactggggaaaatgattaataccataaggagacattactcctcagtcccttacagtggagcgaagacccatgtgcaaccaatctgtatagttcattaaataactataattcatgagtatatagagcaatgtgatctagttgagattaataaatattcactgtatagtctaattaagatggaaaacacaatgaatatttattaattgattttttgggggtaaaatgaccagtattaacattaacatgattgttgggtatcaatactgtcatgaagtgaatttaaaaaatattttatgtgcctgtatcttttccatttcatgaagttctattttgtagctgatctcattcaattctttaaagccattgtaatccaggcatattttgtccaggctggtaaaactgttaccaaaataagttatcctattttgtatttatcaacaactttatgttaaataattataaactttaaatacatataagtccctcttgtttccctgggagtacaactctggatagaatctgatttcacgtaaagcagccccttctctcatgtcattaagaaattaacacacatatagagaatggcgaactgctttaaatatatattttaaaatttgaccatcattttagtatacatggtgatgaaaagtgttacttaagttatatcttttttttttttaataatgttgttttttcagtttgtgattttcgttatttatacgaagagcgagctggttttcttaccaaactggaaacctagctgtttgaactatgatgacatatctaacatattctacctttttggagtttatcttgaaccaagaaaaattatgggaggaaataacagctcattgcttgattaatgattcaaatttttaaaatgtttctcatgaaatgaaagaatggcagatgttaatattgttattatcttaatggccatgactcaattgaccctagaatgagatttcatttgtcacatagcatctgcaaggctgaattttcatgatgccaaccaatctggcacatcttgttttctggcaagctcttctggcctctggcaggtttagcctaatggagcactatccacccaacgtccagtccaacagaggaatcacacattacatgcttcccagagggtacatcctggggctgctttacagctctgctggcaacacaggaacttcccgtccacgaagaacccactatggtacttgaccagcaggtgggggttaccccttatctctgaggagcagacaggaagaaaacaagacgttagcaaacgttgatccaagaggagaaacattcagtaagtgctgttatcacagaacaataaaaaccccatttggccagcaaccaaggtaagagcaaatggttccggaaggatataggtctttcactcaacagttaataagccaaaaagaaaaaagaaaagtctaaccatgagtatgttgagaacaacacaattacatatcatttttgaagaaaaaataactagtaaaatgcatatacagatcagcttgatgttaggattttagagatatatctgtaaagaaagtgagtgggttacaacacatggtgttttcttgtggcagaaggggaagggtggggctaaattttagcaaaaggttctaaaaaaaaggcctgaataaacatacacttccatctactttttattgtgaaaattttcaaatgtacagaaaagttgcaagaataagattaatacaatgagcatccatatgccttttacctagattcaccaattattaactgattgccacatctgctgtatctctttctctctgtacatatgtattattctttaaattttaaaatcattattatattgtgcttaataatttatgaggaagtagtagatggtatgacctaaataatgtcatccataaatgcttcagcacatagcttatattaacaaggatgttcccttatataactgtaatacgattatcaaactcaggaaatttaacactgatacaatattattattattattaatttttaaaattttttaatttttattttactttaagttctgggatacatgtgcagaatgtgcaggtttgttacatagggatacatgtgccatggtggtttgctgcacccatcaacccatcatctaggttttaagccccccatgcattaggtatctgtcctaatgctctcccaccccttgcccctcatcccccaacaggccctggtgtgtgatgttcccctccctgtgtccatgtgttctcattgttcaactgtcacttatcagtgagaacatgcggtgattggttttctgttcctgtgttagtttgctgagaatgatggcttccagcttcatccatgtccctgcaaaggacatgaactcattcttttttatggctgcatttattatttaaaatatatttcatctttaaaatttttctaattattccaataatggcctttataatcactcactttttttctgatcaaggatccaattcaaaatcaaacatggcacttagtttcccttaatttagaactgcttctcatcccttccttgacttctgtgactttgacatgtttatactaaggaatgaatggtttatggggctactttcctaggaatatgtgcatattttaaaaagccacgggaatagggcaatgtataattcaaataaatgaaactgtaatctataacaaattagcctcttttgaaatacaacagagaaacctacaagatcttagtatattttaagaagagtatctaggtgagaagggctggttctatatgtgcctttaactctttctctcttgaccttatgttcatcttgagatttccttatgttgatcccaggagagttatgcttatccttacagtcctgactcaaggaagcgtatggagataataattactttgctgggtcattgggaggctgttagataatgtatgtaaagcacccagcagtgtttggcccagagtaagtaggtgttcaataaacagtgggctttagtgatatcattcccttaatttaacttagctcagtttagctgaaaatgctggcattaaaaaactttactaatgagaattaaagggaaattatgacagaaatcacaaaaggcaaaaagagttaaaattataaaacgtaagtttctgattttggttccaaatggccattgcaatctcttgggcacattacttgccttcctgctctctaagcttctggcctccacatgttgggattcctattatttccttactacatggatttggtttcctgggtaactaattctttttccaaccaaatccaaaagtctacattgcaacagtctcaccacctaataacgacaataagaggaactcatatattcatatatccataggactttacatttatgtattttcctttacatatgtcctctaatttaatcaattcaagaacctgaggggtagctattgttattacacctctcacagagttaggtaaactgaagtgcagagaggtagtacaattgctcaacttcacaagcttaataaatagcagacctgagactcagcctcaggctttttctactacattatgatgtacaggtgccaactcctctgcaaaacccccttctgttttaatttttggaaggaagagcacattttttccacatctggttagcgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat

SEQ ID NO: 2 - protein, 805 aa, Homo sapiens [sequence omitted]

SEQ ID NO: 3 - protein, 805 aa, Homo sapiens [sequence omitted]

SEQ ID NO: 4 - protein, 555 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLL

SEQ ID NO: 5 - protein, 208 aa, Homo sapiens
QPFLLRNGANEGFHEAVGEIMSLSAATPKHLKSIGLLSPDFQEDNETEINFLLKQALTIVGTLPFTYMLEKWRWMVFKGEIPKDQWMKKWWEMKTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKA

SEQ ID NO: 6 - protein, 194 aa, Homo sapiens
MFFLTITSSHCLPSPMSLYPVILSLISFSPAGEVFILLIVSSVPQVKMFVLFLYREEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 7 - protein, 135 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNP

SEQ ID NO: 8 - protein, 26 aa, Homo sapiens
MMDMLICFTIVTILLYKYLITSCCTP

(19) United States
(12) Patent Application Publication
(10) Pub. No.: US 2024/0423176 A1
(43) Pub. Date: Dec. 26, 2024

(54) FULL-LENGTH HUMAN ACE2 MOUSE MODEL SUSCEPTIBLE TO EMERGING CORONAVIRUSES

(71) Applicant: Board of Supervisors of Louisiana State University and Agricultural and Mechanical College, Baton Rouge, LA (US)

(72) Inventors: Xiaohong LU, Shreveport, LA (US); Xinli TIAN, Shreveport, LA (US)

(21) Appl. No.: 18/667,663

(22) Filed: May 17, 2024

Related U.S. Application Data
(63) Continuation of application No. 17/409,265, filed on Aug. 23, 2021, now abandoned.
(60) Provisional application No. 63/063,418, filed on Aug. 9, 2020.

(51) Int. Cl. A01K 67/0278; A01K 67/0276; C12N 15/85

(52) U.S. Cl. CPC A01K 67/0278; A01K 67/0276; C12N 15/8509; A01K 2207/15; A01K 2217/072; A01K 2217/206; A01K 2227/105; A01K 2267/0337; C12N 2015/8581

(57) ABSTRACT

A genetically modified non-human animal comprising a genome containing an endogenous non-human ACE2 locus genetically modified to encode a complete human ACE2 gene. According to a further embodiment the genome is genetically modified to encode a second, a third, and a fourth complete human ACE2 gene, the human ACE2 gene is at least 85 percent identical to SEQ ID No: 1, the animal of is a mouse, the human ACE2 gene encodes six protein variants, an endogenous Tmprss2 gene is unmodified, a LoxP gene flanks each of a 5′ and a 3′ end of a nucleic acid sequence of the human ACE2 gene, and the human ACE2 gene is expressed in a lung, kidney, spleen, stomach, liver, intestine, heart, and skeletal muscle of the animal, and a cortex, striatum, middle brain, hippocampus, olfactory bulb, and cerebellum of a brain of the animal.

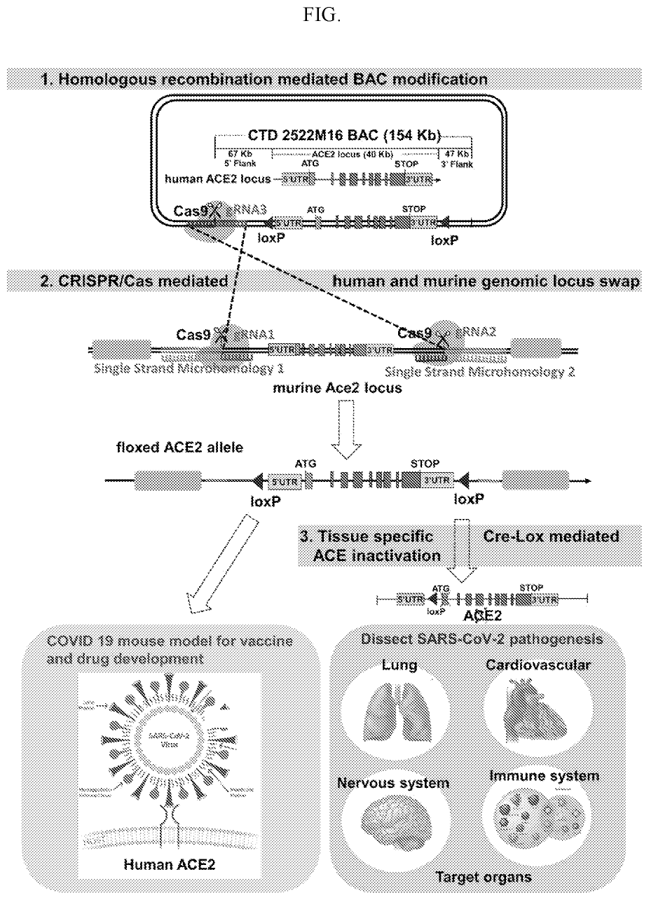

### CROSS REFERENCE TO RELATED APPLICATIONS/PRIORITY

The present invention claims priority to U.S. Provisional Patent Application No. 63/063,418 filed Aug. 9, 2020, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### REFERENCE TO A SEQUENCE LISTING

The application includes sequences in a txt file named p115_ST25.txt, created Aug. 23, 2021 and of 234,254 bytes, which is incorporated by references.

### BACKGROUND

COVID-19 is rapidly spreading around the world and constitutes a global health crisis. This disease is caused by the SARS-COV-2 virus, a coronavirus related to the SARS-COV virus, which caused the global SARS outbreak in 2003. The increasing global spread of SARS-COV-2 promotes the urgent need for coronavirus vaccines and therapeutics. However, the efforts to develop medical countermeasures are hindered by the lack of fundamental understanding of SARS-COV-2 and COVID-19, how it is transmitted, host immunity, disease immunopathogenesis, and the genetic, immunologic, and clinical associations with more severe disease outcomes. Developing animal models is a vital early step toward understanding disease pathogenesis and testing the efficacy of medical countermeasures. Therefore, the very first research priority in the NIAID Strategic Plan for COVID-19 emphasizes the development of small and large animal models that replicate human disease (Objective 1.3).

For the preclinical evaluation, non-human primates (NHPs) are instrumental but are restricted by high costs, availability, and the complexity of husbandry facilities. Thus, appropriate small animal models are essential for pathogenesis research and antiviral therapeutic development. However, it has been demonstrated that SARS COV-2 uses human, bat, or civet ACE2 as a cellular receptor, but not the mouse Ace2. To improve the virus inoculation, some transgenic lines of mice were created using conventional transgenic technologies that expressing human ACE2 under the control of a lung ciliated epithelial cell-specific HFH4/FOXJI promoter (HFH4-hACE2 in C3B6 mice) or human cytokeratin 18 (K18) promoter in epithelial cells, or the CAG promoter with CMV-IE enhancer or a mouse promoter. These mice expressed high levels of hACE2 in the lung but at varying expression levels in other tissues, including the brain, liver, kidney, and gastrointestinal tract. Some of the mice require SARS-COV contagion in high viral load or demonstrated a clinical picture of lethal encephalitis and pneumonia, resulting in death after 3-5 days of the post-inoculation period. In all the above transgenic mice, neuroinvasion is a common pathologic outcome following SARS-CoV infection and a major driver of mortality, which is not quite representative of the human clinical picture. Ralph Baric lab generated a recombinant SARS COV-2 with reverse-engineered spike protein that can use murine Ace2 for entry. However, this model has an incorrigible drawback. The mutation of the receptor-binding domain (RBD) of the spike protein incapacitates any effort using this model to evaluate human monoclonal antibodies, vaccines, and therapeutics, which focuses on targeting the specific domain.

### SUMMARY

Wherefore, it is an object of the present invention to overcome the above-mentioned shortcomings and drawbacks associated with the current technology.

The presently disclosed invention relates to a genetically modified non-human animal comprising a genome containing an endogenous non-human ACE2 locus genetically modified to encode a complete human ACE2 gene. According to a further embodiment the genome is genetically modified to encode a second complete human ACE2 gene. According to a further embodiment the genome is genetically modified to encode a third complete human ACE2 gene and forth complete human ACE2 gene. According to a further embodiment the animal is a rodent. According to a further embodiment the rodent is a mouse. According to a further embodiment the ACE2 gene encodes more than one ACE2 protein. According to a further embodiment the more than one ACE2 protein includes at least one protein having an amino acid sequence that is at least 85 percent identical to one of SEQ ID No:2 to SEQ ID No:8. According to a further embodiment the more than one ACE2 protein includes a first protein and a second protein, each having an amino acid sequence that is at least 85 percent identical to a respective sequence chosen from the group of SEQ ID No:2 to SEQ ID No:8. According to a further embodiment the more than one ACE2 protein includes a third protein having an amino acid sequence that is at least 85 percent identical to a respective sequence chosen from the group of SEQ ID No:2 to SEQ ID No:8. According to a further embodiment the more than one ACE2 protein includes a fourth protein having an amino acid sequence that is at least 85 percent identical to a respective sequence chosen from the group of SEQ ID No:2 to SEQ ID No:8. According to a further embodiment the more than one ACE2 protein includes a sixth protein having an amino acid sequence that is at least 85 percent identical to a respective sequence chosen from the group of SEQ ID No:2 to SEQ ID No:8. According to a further embodiment the genome is genetically modified to encode four complete human ACE2 genes, and an endogenous Tmprss2 gene is unmodified. According to a further embodiment a LoxP gene flanks each of a 5′ and a 3′ end of a nucleic acid sequence of the human ACE2 gene. According to a further embodiment the human ACE2 gene is expressed in a lung, kidney, spleen, stomach, liver, intestine, heart, and skeletal muscle of the animal. According to a further embodiment the human ACE2 gene is expressed in a brain of the animal. According to a further embodiment the human ACE2 gene is expressed in a cortex, striatum, middle brain, hippocampus, olfactory bulb, and cerebellum of the brain of the animal. According to a further embodiment the genome is genetically modified to encode a second, a third, and a fourth complete human ACE2 gene, the human ACE2 gene is at least 85 percent identical to SEQ ID No: 1, the animal of is a mouse, the human ACE2 gene encodes six protein variants, an endogenous Tmprss2 gene is unmodified, a LoxP gene flanks each of a 5′ and a 3′ end of a nucleic acid sequence of the human ACE2 gene, and the human ACE2 gene is expressed in a lung, kidney, spleen, stomach, liver, intestine, heart, and skeletal muscle of the animal, and a cortex, striatum, middle brain, hippocampus, olfactory bulb, and cerebellum of a brain of the animal.

The presently disclosed invention further relates to a transgenic rodent cell comprising a genome containing an endogenous non-human ACE2 locus genetically modified to encode a complete human ACE2 gene, the genome is genetically modified to encode a second complete human ACE2 gene, the human ACE2 gene is at least 85 percent identical to SEQ ID No: 1, the human ACE2 gene encodes multiple protein variants, an endogenous Tmprss2 gene is unmodified, and a LoxP gene flanks each of a 5′ and a 3′ end of a nucleic acid sequence of the human ACE2 gene. According to a further embodiment the cell is a zygote.

The presently disclosed invention further relates to methods of creating a transgenic non-human animal comprising microinjecting into fertilized mouse oocytes a) a gene sequence that is at least 90 percent identical to SEQ ID No: 1, b) Cas9-ribonuclear protein (RNP), c) multiple gRNAs targeting mouse endogenous Ace2 locus, and d) multiple single-stranded oligonucleotides.

A genetically modified non-human animal (e.g., a rodent, e.g., a mouse or a rat) is provided that comprises in its genome a nucleic acid sequence encoding a human ACE2 (hACE2) protein. Thus, genetically modified non-human animals that express human ACE2 are provided. Also provided herein is a model for preclinical testing of hACE2-based therapeutics, e.g., treatments for and vaccines against SARS-COV, SARS-COV-2, and emerging coronaviruses.

In some embodiments, the genetically modified non-human animal described herein comprises a nucleic acid sequence encoding a human ACE2 protein operably linked to an hACE2 promoter.

In some embodiments, the genetically modified non-human animal described herein comprises a nucleic acid sequence encoding a human ACE2 protein with a LoxP gene flanking each of the 5′ and 3′ ends of the nucleic acid sequence.

In one embodiment of the method, the human ACE2 genetic sequences are inserted in a single embryonic stem cell (ES cell), and the single ES cell is introduced into the mouse embryo to make a mouse.

The present invention relates to an animal model and methods to develop an animal model that replicates human disease, and aids in understanding disease pathogenesis and testing the efficacy of medical countermeasures.

Another object of the present invention is for the identification of therapeutics and enabling further steps in advancing toward clinical testing for SARS-COV, SARS-CoV-2, and emerging coronaviruses.

A further object of the invention is for the identification of drug candidates and therapeutics and enabling further steps in advancing vaccines toward clinical testing for SARS-COV, SARS-COV-2, and emerging coronaviruses.

A still further object of the invention is to evaluate the safety of vaccines and therapeutics against SARS-COV, SARS-COV-2, and emerging coronaviruses.

Disclosed herein is a novel full length ACE2 BAC transgenic mouse model susceptible to SARS-COV, SARS-COV-2, and emerging coronaviruses.

Further disclosed herein is a mouse model for identification vaccine and enabling the next step in advancing toward clinical testing for SARS-COV, SARS-COV-2, and emerging coronaviruses.

Further disclosed herein is a mouse model for identifying drug candidates and therapeutics and enabling the next step in advancing vaccine toward clinical testing for SARS-COV, SARS-COV-2, and emerging coronaviruses

Further disclosed herein is a mouse model for evaluating the safety of the vaccine and therapeutics against SARS-COV, SARS-COV-2, and emerging coronaviruses

Further disclosed herein is a novel method to examine the infectivity of SARS-CoV, SARS-COV-2, and emerging coronaviruses in vivo in a full-length ACE2 transgenic mouse model

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which the single FIGURE is a schematic representation of a design and critical steps to generate the humanized transgenic mouse model and its applications according to an embodiment of the presently disclosed invention.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures. are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

In addition, the invention does not require that all the advantageous features and all the advantages of any of the embodiments need to be incorporated into every embodiment of the invention.

Turning now to the FIGURE, a brief description concerning the various components of the present invention will now be briefly discussed.

The increasing global spread of SARS-COV-2 promotes the urgent need for coronavirus vaccines and therapeutics. However, the efforts to develop medical countermeasures is hindered by the lack of fundamental understanding of SARS-COV-2 and COVID-19. Developing animal models that replicate human disease is a vital early step toward understanding disease pathogenesis and testing the efficacy of medical countermeasures. Current transgenic models of human ACE2 are limited by promoters and conventional transgenic design and are not appropriate to study pathogenesis and tissue tropism. It is very important to establish a COVID-19 murine model not only to be able to be infected by the SARS-COV-2 but also for the infection process to be as human-like as possible. Therefore, the inventors invented and generated a full-length human ACE2 transgenic mouse model with all the human ACE2 regulatory regions to faithfully replicate the structure, tissue distribution, and gene regulation of human ACE2. The disclosed invention relates to establishing a small animal model that is susceptible to SARS-COV, SARS-COV-2, and emerging coronaviruses. The model is vital for the identification of COVID-19 therapeutics and enabling the next step in advancing vaccine or drug candidates toward clinical testing and is critical to evaluate the safety of the vaccine and therapeutics. Without an animal model that closely replicates what goes on in humans, there's potential for harm in a fast-moving pandemic response like the one mobilizing now.

The inventors contemplated the small animal models currently available and noted their deficiencies. First, all of the current Ace2 transgenic models are limited by their promoters and are not appropriate to study pathogenesis and tropism. Second, the poor tropism of SARS COV-2 in mouse tissues suggests mouse Ace2 has a difference in protein structure and possible tissue distribution and gene regulation compared to human ACE2. Third, since the transgenic human ACE2 can facilitate SARS COV-2 transduction in mice, suggesting mouse Tmprss2, another crucial protein for SARS CoV-2 cell-entry, is still functional, so there is no need to co-express a human TMPRSS with ACE2. Finally, the neuroinvasiveness in the mice using the mouse promoter suggests the tissue distribution of mouse Ace2 is different from the human counterpart, highly likely in the cis transcriptional regulatory region. The inventors concluded that it is important not only for the COVID-19 murine model to be able to be infected by the SARS-COV-2 but also for the infection process to be as human-like as possible. In response to the inventors' conclusions, they generated a CRISPR/Cas mediated humanized transgenic mouse model with full-length human ACE2 regulatory regions to faithfully replicate structure, tissue distribution, and gene regulation of human ACE2.

The disclosed mouse model is believed to be inventively novel in comparison to all the human ACE2 transgenic mouse models of COVID19 that existent or in that are known to be in the pipelines.

Large transgene size for a stable human ACE2 gene expression: All the current technology models that the inventors are aware of were generated using conventional transgenic technologies with the limited size of the transgene (<20 kb). Such conventional transgenes are often plagued by inconsistency in expression due to positional effects, which refer to the phenomena of variable, ectopic, or complete lack of transgene expression due to influence from the genomic locus at which the transgene integrated. In contrast, the inventors' full-length human ACE2 mouse model encompasses the entire repertoire of regulatory elements for human ACE2, overcoming positional effects and conferring accurate, dosage-dependent, integration-site independent, and tissue-specific human ACE2 transgene expression in vivo.

Full-length promoter for a faithful and human tissue-specific ACE2 gene expression: Current human ACE2 transgenic mice the inventors are aware of that are available were generated using viral or truncated tissue-specific promoters to drive the expression of a human cDNA ACE2. These promoters are not believed to be able to be used to replicate specific human ACE2 expressions. Such conventional transgenes, with an average insert size of about 20 kb, are often plagued by inconsistency in expression due to positional effects, which refer to the phenomena of variable, ectopic, or complete lack of transgene expression due to influence from the genomic locus at which the transgene integrated. An average mammalian gene locus spans about 30 kb of genomic DNA, but the critical transcription regulatory gene elements could be located upstream of or downstream to the coding region or may also be located in the introns. Thus, the inventors concluded that Bacterial Artificial Chromosome (BAC) transgenesis that employs large genomic inserts of 100-200 kb, is needed to encompass the entire repertoire of regulatory elements for a given gene to overcome positional effects and confer accurate, dosage-dependent, integration-site independent transgene expression in vivo.

Human ACE2 spans about 40 kb of a genomic region on Chromosome X, with 19 exons, 21 introns, 3 alternative promoters, and 7 alternative transcripts (splice variants, 6 encodes proteins).

Angiotensin-converting enzyme 2 (92.5 kD) (ACE2) alternative variant aAug10: This complete mRNA is 3458 bp long. It is reconstructed from 2 cDNA clones, some from testis (seen 2 times). The premessenger has 18 exons and covers 40.05 kb on the NCBI 37, August 2010 genome. The predicted protein has 805 aa (92.5 kDa, pI 6.0), SEQ ID No:2. It is exactly encoded by AB193260. It contains one Angiotensin-converting enzyme domain [Pfam], a transmembrane domain [Psort].

Angiotensin-converting enzyme 2 (92.5 kD) (ACE2) alternative variant bAug10: This complete CDS mRNA is 3593 bp long. It is reconstructed from 239 cDNA clones, some from testis (seen 175 times), kidney (15), small intestine (10), testis normal (6), placenta (4), colon (3), ilea mucosa (3) and 12 other tissues. The premessenger has 19 exons and covers 41.12 kb on the NCBI 37, August 2010 genome. The predicted protein has 805 aa (92.5 kDa, pI 6.0), SEQ ID No:3. It contains one Angiotensin-converting enzyme domain [Pfam], a transmembrane domain [Psort2].

Angiotensin-converting enzyme 2 precursor (63.9 kD) (ACE2) alternative variant cAug10: This complete mRNA is 3723 bp long. It is reconstructed from one cDNA clone. This mRNA could be a target of nonsense mediated mRNA decay. The premessenger has 17 exons and covers 39.91 kb on the NCBI 37, August 2010 genome. The predicted protein has 555 aa (63.9 kDa, pI 5.4), SEQ ID No:4. It contains one Angiotensin-converting enzyme domain [Pfam]. It is predicted to be secreted or extracellular [Psort2].

Putative protein (ACE2) alternative variant dAug10: This partial mRNA is 625 bp long. It is reconstructed from one cDNA clone from kidney. It may be incomplete at the 5′ end. It is incomplete at the 3′ end. The premessenger has 5 exons and covers 7.93 kb on the NCBI 37, August 2010 genome. The predicted partial protein has 208 aa (24.0 kDa, pI 7.5), SEQ ID No:5.

Angiotensin converting enzyme 2 (21.7 kD) (ACE2) alternative variant eAug10: This complete CDS mRNA is 1426 bp long. It is reconstructed from 3 cDNA clones, some from brain (seen once), ileal mucosa (once), medulla (once). This mRNA could be a target of nonsense mediated mRNA decay. The premessenger has 4 exons and covers 5.68 kb on the NCBI 37, August 2010 genome. The predicted protein has 194 aa (21.7 kDa, pI 8.7), SEQ ID No:6. It contains 2 transmembrane domains [Psort]. It contains no Pfam protein domain.

Angiotensin converting enzyme 2 (ACE2) alternative variant fAug10: This partial mRNA is 627 bp long. It is reconstructed from one cDNA clone from cornea from eye. It is incomplete at the 3′ end. The premessenger has 4 exons and covers 9.91 kb on the NCBI 37, August 2010 genome. The predicted partial protein has 135 aa (15.2 kDa, pI 5.3), SEQ ID No:7. It contains one Angiotensin-converting enzyme domain [Pfam].

Putative protein (3.0 kD) (ACE2) alternative variant gAug10-unspliced: This apparently non-coding mRNA is 544 bp long. It is reconstructed from one cDNA clone from testis. The premessenger has a single exon and covers 0.54 kb on the NCBI 37, August 2010 genome. It is potentially noncoding, with a predicted protein having 26 aa (3.0 kDa, pI 5.5), SEQ ID No:8.

Protein products from some of the transcripts are likely candidates of cellular receptors for SARS-COV-2, and may explain the poor tropism in mouse tissues, the mouse having only 3 splice variants. The 154 kb human ACE2 BAC the inventors' selected encompasses the complete set of cis-transcriptional regulatory elements and introns to direct the human ACE2 gene expression. Therefore, the human ACE2 BAC can express multiple alternatively spliced transcripts, while conventional transgenesis practiced in the current technology can only express one pre-selected transcript.

The human full-length ACE2 BAC approach is needed to faithfully replicate endogenous human ACE expression (protein structure, tissue distribution, coding/non-coding variants, splice variants, and gene regulation) to establish a COVID-19 animal model with a better construct and predictive validities for studying vaccines and therapeutics. In the transgenic mice of current technology, neuroinvasion is a common pathologic outcome following SARS-COV infection and a major driver of mortality, which is not quite representative of the human clinical picture. This may be because the current models do not express all six human ACE2 encoded proteins. To the inventors' knowledge, all of the current models can only express a single pre-selected transcript (hACE2 cDNA) and protein.

A balance of construct and predictive validities are ideal for understanding disease pathogenesis and testing the efficacy and safety of medical countermeasures for SARS-CoV, SARS-COV-2, and emerging coronaviruses. The inventors' full-length and humanized human ACE2 BAC transgenic mouse is believed unique in providing a faithful replication of endogenous human ACE expression (protein structure, tissue distribution, coding/non-coding variants, splice variants, and gene regulation) to establish a COVID-19 small animal model with a better construct and predictive validities than any of the existent model.

As shown in the FIGURE, the inventors identified three human BACs, including CTD-2522M16, CTD-2523M16, CH17-203N23 containing the ACE2 genomic sequence through the UCSC genome browser and BAC database search. CTD-2522M16 contains a human ACE2 locus (40 kb), with a 67 kb 5′ flanking and a 47 kb 3′ flanking genomic region, and with no other full-length genes on the BAC (SEQ ID No 1), and was used to create the transgenic mice. Notably, the BAC was flanked by LoxPs. As a result, the full-length human ACE2 mouse model is designed to be a conditional Knockout model in which Cre can switch off the transgene expression in desired temporal and spatial patterns controlled by the genetic introduction of Cre recombinase (see FIGURE).

Maxiprep DNA was prepared from the modified ACE2 BACs. DNA was separated on a pulse-field gel to select the fractions with intact BAC DNA. The purified BACs were then microinjected into fertilized C57/BL6J mouse oocytes, or zygotes, with Cas9-ribonuclear protein (RNP) and three gRNAs targeting the two mouse endogenous Ace2 locus and the PI-SceI site of BAC backbone (pBACe3.6), together with two single-stranded oligonucleotides (ssODNs, 80 bp). The ssODNs ligate the mouse genome sequences and the BAC sequence via a microhomology-mediated end joining (MMEJ). This strategy directs efficient site-specific integration of the BAC vector into the murine Ace2 locus.

The inventors then performed microinjection into fertilized C57/BL6 pronucleus of the fertilized eggs of the C57BL/6J mice to generate FO mice (8 pups).

The pups were genotyped with transgene-specific primers to identify two transgenic founders.

The inventors then crossed the transgenic founders with C57BL/6J wildtype mice to generate F1 mice. The transgene was successfully germline transmitted.

Then inventors then performed rotating breeding to expand the colony rapidly.

The inventors have used six pairs of human ACE2 specific genes to map the full genomic region of human ACE2. The inventors have determined that the integrated human BAC DNAs in the transgenic mice are intact and contain the full length of the human ACE2 genomic region for each transgene copy number.

The inventors have determined the transgene copy number amongst the first and second transgenic lines to be two and four copies, respectively. The transgenic line with four full gene copies delivers more consistent and sufficient transgene expression, superior to the transgenic line with two gene copies.

The inventors confirmed human ACE2 mRNA expression level using three different pairs of human ACE2 exon-specific primers using RT-PCR analysis.

The inventors determined the human transgene expression in the lung, kidney, spleen, stomach, liver, intestine, heart, and muscle. The inventors also determined the brain expression of human ACE2 in the cortex, striatum, middle brain, hippocampus, olfactory bulb, and cerebellum. The human transgene expression pattern in the disclosed mouse models was consistent with the human ACE2 gene expression pattern as previously reported in human studies.

A recombinant pseudotyped r vesicular stomatitis virus (rVSV) was established to contain rSARS-COV-2 Spike protein. The virus was designed to report cell entry via human ACE2, but its replication is limited to one round and can be handled in a BSL-2 facility. The rVSV containing rSARS-COV-2 Spike protein can be propagated in a Vero cell line a with GFP or mCherry. When the pseudotyped viral spike enters into cells, fluorescence protein expression and activity are proportional to the number of cells that are transduced.

The transgenic mice received a nasal inoculation of the pseudotyped virus at 3×10⁴ TCID50 (Median Tissue Culture Infectious Dose).

The inventors observed fluorescence protein expression two weeks after inoculation in multiple organs.

The inventors discovered lung infection and neural dissemination through axonal transport in vivo that replicates features of SARS-COV-2 infection in humans.

Imaging and 3D reconstruction of neurons infected with pseudotyped VSV-Spike-mCherry. These results support the inventors' claim that the inventors have generated a novel full-length ACE2 BAC transgenic mouse model that faithfully replicates the human ACE2 gene expression in mice, which are susceptible to SARS-COV, SARS-CoV-2, and emerging coronaviruses

While the methods disclosed are believed to be preferable, other methods can be used to generate small mammal/mouse models similar to the full-length human ACE2 mouse model of COVID19 as disclosed herein, including BAC transgenic with random integration. Other genome editing technologies beyond the CRISPR/Cas system may be used, such as TALEN or Zinc-finger. Other variants of genome editing enzymes beyond Cas 9 may be used, such as Cas 12. Additional embodiments may use Transposon-mediated Knock-in a large genomic fragment in zygotes, different serotypes of AAV mediated Knock-in a large genomic fragment in zygotes, lentiviral mediated Knock-in a large genomic fragment in zygotes, and adenovirus-mediated Knock-in a large genomic fragment in zygotes.

In one embodiment of the provided mouse model, the ACE2-based antigen-binding protein is an antibody. In one embodiment, the ACE2-based antigen-binding protein is a human antigen-binding protein. Such mouse model may allow testing for efficacy and/or toxicity of the antigen-binding protein in the mouse.

Also provided herein is a method of screening a drug candidate that target an antigen of interest comprising (a) introducing the antigen of interest into a genetically modified mouse comprising an endogenous non-human X chromosome genetically modified to encode a human ACE2 protein, wherein the drug candidate is directed against the human ACE2 and the antigen of interest, and (c) determining if the drug candidate is efficacious in preventing, reducing or eliminating cells characterized by the presence or expression of the antigen of interest. In one embodiment of the method, the mouse does not comprise a functional extracellular domain of the corresponding mouse protein(s). In one embodiment of the method, the human ACE2 is set forth in SEQ ID NO: 1.

In a particular embodiment of the method of screening drug candidates described herein, the step of introducing the antigen of interest into the mouse described herein comprises expressing in the mouse the antigen of interest. In one embodiment, the step of expressing in the mouse the antigen of interest comprises genetically modifying the mouse to express the antigen of interest. In one embodiment, the step of introducing the antigen of interest comprises infecting the mouse with the antigen of interest. In one embodiment of the method, the step of introducing comprises introducing into said mouse a cell expressing the antigen of interest. In various embodiments of the method, a virus or a cell infected with a virus. Thus, in some embodiments of the method, the mouse comprises and infection which is either a viral or bacterial infection. Thus, the antigen of interest can be an infectious disease associated antigen. In one embodiment, the antigen of interest is a viral antigen, and the viral antigen is selected from the group consisting of a coronavirus, SARS-COV, and SARS-COV-2.

In some embodiments of the method of screening drug candidates, the mouse is an immunocompetent mouse. In some embodiments of the method described herein, the antigen of interest is a human antigen of interest.

In some embodiments of the method, the drug candidate is an antibody. In some embodiments, the drug candidate is an antigen-binding protein. In some embodiments, the drug candidate is a bispecific antibody or a bispecific antigen binding protein. In some embodiments, the bispecific antigen binding protein is capable of binding both human ACE2 protein(s) and the antigen of interest.

In other embodiments, the drug candidate is capable of reducing, eliminating, or preventing viral infection as compared to an agent that does not target the antigen of interest. In some such embodiments, the step of determining if the drug candidate is efficacious in preventing, reducing or eliminating cells characterized by the presence or expression of the antigen of interest comprises the measurement of viral titers or fluorescence protein expression.

In yet other embodiments, provided herein is a non-human animal model, e.g., a mouse model, for testing safety, efficacy, and pharmacokinetics of combination drug therapies wherein the combination therapy includes a drug, e.g., an antigen-binding protein, that binds a human ACE2 molecule. Such combination therapies are aimed at targeting specific infections, or other diseases described herein, including those which can benefit from the recruitment and/or activation of T cells.

The present invention provides genetically modified non-human animals, e.g., rodents, e.g., mice or rats, which express human ACE2 protein. The present invention also relates to genetically modified non-human animals that comprise in their genome, e.g., in their germline, genetically modified genetic code encoding human ACE2 proteins. Also provided are embryos, cells, and tissues comprising the same, methods of making the same, as well as methods of using the same. Unless defined otherwise, all terms and phrases used herein include the meanings that the terms and phrases have attained in the art, unless the contrary is clearly indicated or clearly apparent from the context in which the term or phrase is used.

“ACE2,” as used herein, includes angiotensin-converting enzyme 2 is an enzyme attached to the membrane of cells located in, for example, the intestines, kidney, testis, gallbladder, and heart. ACE2 lowers blood pressure by catalyzing the hydrolysis of angiotensin II into angiotensin. ACE2 counters the activity of the related angiotensin-converting enzyme by reducing the amount of angiotensin-II and increasing Ang.

As used herein, “an antibody that binds human ACE2” or an “anti-human ACE2 antibody” includes antibodies and antigen-binding fragments thereof that specifically recognize a single human ACE2 subunit. The antibodies and antigen-binding fragments of the present invention may bind soluble human ACE2 and/or cell surface expressed human ACE2.

The term “conservative,” when used to describe a conservative amino acid substitution, includes substitution of an amino acid residue by another amino acid residue having a side chain R group with similar chemical properties (e.g., charge or hydrophobicity). Conservative amino acid substitutions may be achieved by modifying a nucleotide sequence so as to introduce a nucleotide change that will encode the conservative substitution. In general, a conservative amino acid substitution will not substantially change the functional properties of interest of a protein. Examples of groups of amino acids that have side chains with similar chemical properties include aliphatic side chains such as glycine, alanine, valine, leucine, and isoleucine; aliphatic-hydroxyl side chains such as serine and threonine; amide-containing side chains such as asparagine and glutamine; aromatic side chains such as phenylalanine, tyrosine, and tryptophan; basic side chains such as lysine, arginine, and histidine; acidic side chains such as aspartic acid and glutamic acid; and, sulfur-containing side chains such as cysteine and methionine. Conservative amino acids substitution groups include, for example, valine/leucine/isoleucine, phenylalanine/tyrosine, lysine/arginine, alanine/valine, glutamate/aspartate, and asparagine/glutamine. In some embodiments, a conservative amino acid substitution can be a substitution of any native residue in a protein with alanine, as used in, for example, alanine scanning mutagenesis. In some embodiments, a conservative substitution is made that has a positive value in the PAM250 log-odds scoring matrix disclosed in Gonnet et al. ((1992) Exhaustive Matching of the Entire Protein Sequence Database, Science 256:1443-45), hereby incorporated by reference. In some embodiments, the substitution is a moderately conservative substitution wherein the substitution has a nonnegative value in the PAM250 log-likelihood matrix.

Thus, encompassed by the invention is a genetically modified non-human animal, e.g., rodent, e.g., mouse or rat, expressing a human ACE2 protein(s) comprising conservative amino acid substitutions in the amino acid sequence described herein

One skilled in the art would understand that in addition to the nucleic acid residues encoding human ACE2 proteins described herein, due to the degeneracy of the genetic code, other nucleic acids may encode the polypeptides of the invention. Therefore, in addition to a genetically modified non-human animal that comprises in its genome nucleotide sequences encoding human ACE2 proteins described herein, a non-human animal that comprises in its genome nucleotide sequences that differ from those described herein due to the degeneracy of the genetic code are also provided.

The term “identity” when used in connection with sequence includes identity as determined by a number of different algorithms known in the art that can be used to measure nucleotide and/or amino acid sequence identity. In some embodiments described herein, identities are determined using a ClustalW v. 1.83 (slow) alignment employing an open gap penalty of 10.0, an extend gap penalty of 0.1, and using a Gonnet similarity matrix (MacVector™ 10.0.2, MacVector Inc., 2008). The length of the sequences compared with respect to identity of sequences will depend upon the particular sequences. In various embodiments, identity is determined by comparing the sequence of a mature protein from its N-terminal to its C-terminal. In various embodiments, when comparing a humanized sequence to a human sequence, the human portion of the humanized sequence (but not the non-human portion) is used in making a comparison for the purpose of ascertaining a level of identity between a human sequence and a humanized sequence.

The term “operably linked” includes a juxtaposition wherein the components so described are in a relationship permitting them to function in their intended manner. As such, a nucleic acid sequence encoding a protein may be operably linked to regulatory sequences (e.g., promoter, enhancer, silencer sequence, etc.) so as to retain proper transcriptional regulation. In addition, various portions of the human protein of the invention may be operably linked to retain proper folding, processing, targeting, expression, and other functional properties of the protein in the cell. Unless stated otherwise, various domains of the human protein of the invention are operably linked to each other.

The term “replacement” in reference to gene replacement includes placing exogenous genetic material at an endogenous genetic locus, thereby replacing all or a portion of the endogenous gene with an orthologous or homologous nucleic acid sequence. In one instance, an endogenous non-human gene or fragment thereof is replaced with a corresponding human gene or fragment thereof.

“Functional” as used herein, e.g., in reference to a functional protein, includes a protein that retains at least one biological activity normally associated with the native

protein. The term “locus” as in human ACE2 locus includes the genomic DNA comprising a human ACE2 coding region.

The term “germline” in reference to a nucleic acid sequence includes a nucleic acid sequence that can be passed to progeny.

The phrase “immunoglobulin molecule” includes two immunoglobulin heavy chains and two immunoglobulin light chains. The heavy chains may be identical or different, and the light chains may be identical or different.

The term “antibody”, as used herein, includes immunoglobulin molecules comprising four polypeptide chains, two heavy (H) chains and two light (L) chains inter-connected by disulfide bonds. Each heavy chain comprises a heavy chain variable domain and a heavy chain constant region (CH). The heavy chain constant region comprises three domains, C_{H}1, C_{H}2 and C_{H}3. Each light chain comprises a light chain variable domain and a light chain constant region (CL). The heavy chain and light chain variable domains can be further subdivided into regions of hypervariability, termed complementarity determining regions (CDR), interspersed with regions that are more conserved, termed framework regions (FR). Each heavy and light chain variable domain comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3).

The term “high affinity” antibody refers to an antibody that has a K_{D} with respect to its target epitope about of 10⁻⁹M or lower (e.g., about 1×10⁻⁹ M, 1×10⁻¹⁰ M, 1×10⁻¹¹ M, or about 1×10⁻¹² M).

The phrase “bispecific antibody” includes an antibody capable of selectively binding two epitopes. Bispecific antibodies generally comprise two arms, each binding a different epitope (e.g., two heavy chains with different specificities)—either on two different molecules (e.g., different epitopes on two different immunogens) or on the same molecule (e.g., different epitopes on the same immunogen). If a bispecific antibody is capable of selectively binding two different epitopes (a first epitope and a second epitope), the affinity of the first antibody arm for the first epitope will generally be at least one to two or three or four or more orders of magnitude lower than the affinity of the first antibody arm for the second epitope, and vice versa. Epitopes specifically bound by the bispecific antibody can be on the same or a different target (e.g., on the same or a different protein). Bispecific antibodies can be made, for example, by combining heavy chains that recognize different epitopes of the same immunogen. For example, nucleic acid sequences encoding heavy chain variable sequences that recognize different epitopes of the same immunogen can be fused to nucleic acid sequences encoding the same or different heavy chain constant regions, and such sequences can be expressed in a cell that expresses an immunoglobulin light chain. A typical bispecific antibody has two heavy chains each having three heavy chain CDRs, followed by (N-terminal to C-terminal) a C_{H}1 domain, a hinge, a C_{H}2 domain, and a C_{H}3 domain, and an immunoglobulin light chain that either does not confer epitope-binding specificity but that can associate with each heavy chain, or that can associate with each heavy chain and that can bind one or more of the epitopes bound by the heavy chain epitope-binding regions, or that can associate with each heavy chain and enable binding of one or both of the heavy chains to one or both epitopes. Similarly, the phrase “multispecific antibody” includes an antibody capable of selectively binding multiple epitopes (e.g., two, three, four epitopes).

The phrase “complementarity determining region,” or the term “CDR,” includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule. A CDR can be encoded by, for example, a germline sequence or a rearranged or unrearranged sequence, and, for example, by a naive or a mature B cell. A CDR can be somatically mutated (e.g., vary from a sequence encoded in an animal's germline), humanized, and/or modified with amino acid substitutions, additions, or deletions. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

The phrase “functional fragment” includes fragments of antigen-binding proteins such as antibodies that can be expressed, secreted, and specifically bind to an epitope with a KD in the micromolar, nanomolar, or picomolar range. Specific recognition includes having a KD that is at least in the micromolar range, the nanomolar range, or the picomolar range.

The phrase “heavy chain,” or “immunoglobulin heavy chain” includes an immunoglobulin heavy chain sequence, including immunoglobulin heavy chain constant region sequence, from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a C_{H}1domain, a hinge, a C_{H}2 domain, and a C_{H}3 domain. A functional fragment of a heavy chain includes a fragment that is capable of specifically recognizing an epitope (e.g., recognizing the epitope with a K_{D} in the micromolar, nanomolar, or picomolar range), that is capable of expressing and secreting from a cell, and that comprises at least one CDR. A heavy chain variable domain is encoded by a variable region gene sequence, which generally comprises V_{H}, D_{H}, and J_{H} segments derived from a repertoire of V_{H}, D_{H}, and J_{H} segments present in the germline. Sequences, locations and nomenclature for V, D, and J heavy chain segments for various organisms can be found on the website for the International Immunogenetics Information System (IMGT database).

The phrase “light chain” includes an immunoglobulin light chain sequence from any organism, and unless otherwise specified includes human kappa and lambda light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region. A light chain variable domain is encoded by a light chain variable region gene sequence, which generally comprises V_{L} and J_{L} segments, derived from a repertoire of V and J segments present in the germline. Sequences, locations and nomenclature for V and J light chain segments for various organisms can be found on the website for the International Immunogenetics Information System (IMGT database). Light chains include those, e.g., that do not selectively bind any epitopes recognized by antigen-binding protein (e.g., antibody) in which they appear. Light chains also include those that bind and recognize, or assist the heavy chain with binding and recognizing, one or more epitopes selectively bound by the antigen-binding protein (e.g., an antibody) in which they appear.

The term “antigen-binding protein” as used herein includes antibodies and various naturally produced and engineered molecules capable of binding the antigen of interest. Such include, e.g., domain-specific antibodies, single domain antibodies (e.g., derived from camelids and fish, etc.), domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), shark variable IgNAR domains, etc. Antigen-binding protein may also include antigen-binding fragments such as, e.g., (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), etc.

The term “cell” includes any cell that is suitable for expressing a recombinant nucleic acid sequence. Cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, the cell comprises one or more viral genes, e.g. a retinal cell that expresses a viral gene (e.g., a PER.C6™ cell). In some embodiments, the cell is an ES cell.

The terms “polypeptide” and “peptide” and “protein” are used interchangeably herein and include polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies or other members of the immunoglobulin superfamily, in certain embodiments, a “polypeptide” can occur as a single chain or as two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 nucleotides or amino acid residues, at least about 60-80 nucleotides or amino acid residues in length or any integral value there between. In some embodiments, identity exists over a longer region than 60-80 nucleotides or amino acid residues, such as at least about 80-100 nucleotides or amino acid residues, and in some embodiments the sequences are substantially identical over the full length of the sequences being compared, for example, the coding region of a nucleotide sequence.

A “conservative amino acid substitution” includes one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Generally, conservative substitutions in the sequences of polypeptides and/or antibodies of the invention do not abrogate the binding of the polypeptide or antibody containing the amino acid sequence, to the target binding site. Methods of identifying nucleotide and amino acid conservative substitutions which do not eliminate binding are well-known in the art.

The term “vector” as used herein includes a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

The term “substantially pure” as used herein includes material which is at least 50% pure (i.e., free from contaminants), at least 90% pure, at least 95% pure, at least 98% pure, or at least 99% pure.

The term “immune response” as used herein includes responses from both the innate immune system and the adaptive immune system. It includes both cell-mediated and/or humoral immune responses. It includes, but is not limited to, both T-cell and B-cell responses, as well as responses from other cells of the immune system such as natural killer (NK) cells, monocytes, macrophages, etc.

The term “subject” includes any animal (e.g., a mammal), including, but not limited to, humans, non-human primates, canines, felines, rabbits, rodents, and the like, which is to be the recipient of a particular treatment. Typically, the terms “subject” and “patient” are used interchangeably herein in reference to a human subject.

The term “pharmaceutically acceptable” includes a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier, or adjuvant” or “acceptable pharmaceutical carrier” includes an excipient, carrier, or adjuvant that can be administered to a subject, together with at least one agent of the present disclosure, and which does not destroy the pharmacological activity thereof and is non-toxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The terms “effective amount” or “therapeutically effective amount” or “therapeutic effect” includes an amount of an agent, an antibody, a polypeptide, a polynucleotide, a small organic molecule, or other drug effective to “treat” a disease or disorder in a subject such as, a mammal. In the case of cancer or a tumor, the therapeutically effective amount of an agent (e.g., polypeptide or antibody) has a therapeutic effect and as such can enhance or boost the immune response, enhance or boost the anti-tumor response, increase cytolytic activity of immune cells, increase killing of tumor cells, increase killing of tumor cells by immune cells, reduce the number of tumor cells: decrease tumorigenicity, tumorigenic frequency, or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis: inhibit and stop tumor or cancer cell growth; relieve to some extent one or more of the symptoms associated with the cancer; reduce morbidity and mortality; improve quality of life; or a combination of such effects.

The terms “treating” or “treatment” or “to treat” or “alleviating” or “to alleviate” includes both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented. In the case of cancer or a tumor, a subject is successfully “treated” according to the methods of the present invention if the patient shows one or more of the following: an increased immune response, an increased anti-tumor response, increased cytolytic activity of immune cells, increased killing of tumor cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth: relief of one or more symptoms associated with the specific cancer, reduced morbidity and mortality; improvement in quality of life: reduction in tumorigenicity: reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

As used herein, reference to “about” or “approximately” a value or parameter includes (and describes) embodiments that are directed to that value or parameter. For example, description referring to “about X” includes description of “X”. About or approximately or substantially can include +/−5% of a stated value. For example, “about 1.0,” “approximately 1.0,” or “substantially 1.0,” includes 0.95 to 1.05,inclusive.

The term “and/or” as used in a phrase such as “A and/or B” herein is intended to include both A and B: A or B: A (alone); and B (alone). Likewise, the term “and/or” as used in a phrase such as “A. B, and/or C” is intended to encompass each of the following embodiments: A. B, and C:A, B, or C:A or C; A or B:B or C:A and C:A and B:B and C:A (alone); B (alone); and C (alone).

Genetically Modified Human ACE2 Animals: In various embodiments, the present invention provides genetically modified non-human animals (e.g., rodents, e.g., mice or rats) that comprise in their genome (e.g., in their germline genome) a nucleic acid sequence encoding a human ACE2 protein.

In some embodiments of the invention, the non-human animal is a mammal. In one aspect, the non-human animal is a small mammal, e.g., of the superfamily Dipodoidea or Muroidea. In one embodiment, the genetically modified animal is a rodent. In one embodiment, the rodent is selected from a mouse, a rat, and a hamster. In one embodiment, the rodent is selected from the superfamily Muroidea. In one embodiment, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, white-tailed rats, Malagasy rats and mice), Platacanthomyidae (e.g., spiny dormice), and Spalacidae (e.g., mole rats, bamboo rats, and zokors). In a specific embodiment, the genetically modified rodent is selected from a true mouse or rat (family Muridae), a gerbil, a spiny mouse, and a crested rat. In one embodiment, the genetically modified mouse is from a member of the family Muridae. In one embodiment, the animal is a rodent. In a specific embodiment, the rodent is selected from a mouse and a rat. In one embodiment, the non-human animal is a mouse.

In one embodiment, the non-human animal is a rodent that is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/O1a. In another embodiment, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2 (see, e.g., Festing et al. (1999) Revised nomenclature for strain 129 mice, Mammalian Genome 10:836, see also, Auerbach et al (2000) Establishment and Chimera Analysis of 129/SvEv-and C57BL/6-Derived Mouse Embryonic Stem Cell Lines). In a specific embodiment, the genetically modified mouse is a mix of two of the aforementioned strains

In one embodiment, the non-human animal is a rat. In one embodiment, the rat is selected from a Wistar rat, an LEA strain, a Sprague Dawley strain, a Fischer strain, F344, F6, and Dark Agouti. In one embodiment, the rat strain is a mix of two or more strains selected from the group consisting of Wistar, LEA, Sprague Dawley, Fischer, F344, F6, and Dark Agouti.

Thus, in one embodiment, the genetically modified non-human animal is a rodent. In one embodiment, the genetically modified non-human animal is a rat or a mouse. In one embodiment, the animal is a mouse. Thus, in one embodiment, the genetically modified animal is a mouse and the mouse comprises at one, two, three, four, five or more locations on a nucleotide sequence encoding a human ACE2 protein.

In some embodiments of the invention, the mouse of the invention expresses human ACE2 protein(s).

Also provided herein are methods of making the genetically modified non-human animal described herein. In some embodiments, the method of making a genetically modified non-human animal wherein the animal expresses a human ACE2 protein comprises introducing at between 1 and 10 locations on a one or more chromosomes a nucleic acid sequence encoding a human ACE2 protein. In further embodiments, all locations of the nucleic acid sequence insertion are on the X chromosome. In further embodiments, if multiple human ACE2 proteins are introduced, they can be introduced together on a single nucleic acid or separately. If the latter, a single cell line (e.g., ES cell line) can undergo successive modifications until modified to include nucleic acids encoding each of the desired number of human ACE2 proteins.

Nucleic acid(s) encoding human ACE2 protein(s) may be introduced into a cell, and a non-human animal is propagated from the cell. In some embodiments, the replacement method utilizes a targeting construct made using VELOCIGENE® technology, introducing the construct into ES cells, and introducing targeted ES cell clones into a mouse embryo using VELOCIMOUSE® technology.

A selection cassette is a nucleotide sequence inserted into a targeting construct to facilitate selection of cells (e.g., bacterial cells, ES cells) that have integrated the construct of interest. A number of suitable selection cassettes are known in the art (Neo, Hyg, Pur, CM, SPEC, etc.). In addition, a selection cassette may be flanked by recombination sites, which allow deletion of the selection cassette upon treatment with recombinase enzymes. Commonly used recombination sites are loxP and Frt, recognized by Cre and Flp enzymes, respectively, but others are known in the art. A selection cassette may be located anywhere in the construct outside the coding region. In one embodiment, the selection cassette is inserted upstream of human ACE2 inserted sequence.

Upon completion of gene targeting, ES cells or genetically modified non-human animals may be screened to confirm successful incorporation of exogenous nucleotide sequence of interest or expression of exogenous polypeptide. Numerous techniques are known to those skilled in the art, and include (but are not limited to) Southern blotting, long PCR, quantitative PCR (e.g., real-time PCR using TAQMAN®), fluorescence in situ hybridization, Northern blotting, flow cytometry, Western analysis, immunocytochemistry, immunohistochemistry, etc. In one example, non-human animals (e.g., mice) bearing the genetic modification of interest can be identified by screening for loss of mouse allele and/or gain of human allele using a modification of allele assay described in Valenzuela et al. (2003) High-throughput engineering of the mouse genome coupled with high-resolution expression analysis, Nature Biotech. 21 (6): 652-659. Other assays that identify a specific nucleotide or amino acid sequence in the genetically modified animals are known to those skilled in the art.

Heterozygotes resulting from the above methods can be bred to generate homozygotes.

In one aspect, a non-human cell that expresses a human ACE2 protein is provided. In one embodiment, the cell comprises an expression vector comprising a human ACE2 sequence as described herein. In one embodiment, the cell is selected from CHO, COS, 293, HeLa, and a retinal cell expressing a viral nucleic acid sequence (e.g., a PERC.6™ cell).

In addition to a genetically engineered non-human animal, a non-human embryo (e.g., a rodent, e.g., a mouse or a rat embryo) is also provided that comprises a human ACE2 gene. In one embodiment, the embryo comprises a donor ES cell that is derived from a non-human animal (e.g., a rodent, e.g., a mouse or a rat) as described herein. In one aspect, the embryo comprises an ES donor cell that comprises the human ACE2 gene, and host embryo cells.

Also provided is a tissue, wherein the tissue is derived from a non-human animal (e.g., a rodent, e.g., a mouse or a rat) as described herein, and expresses the human ACE2 proteins.

In addition, a non-human cell isolated from a non-human animal as described herein is provided. In one embodiment, the cell is an ES cell.

In some embodiments, also provided herein are genetic loci comprising the nucleic acid sequences that encoding the human ACE2 protein(s) described herein.

Mouse Model for Testing Human Therapies. In some aspects, provided herein is a mouse model for testing human ACE2-targeted (“anti-ACE2”) therapeutic agents. In some embodiments, provided herein is a mouse model for testing anti-ACE2 antigen-binding proteins. In some embodiments, provided herein is a mouse model for testing anti-ACE2 antibodies. In some such embodiments, provided is a mouse model for testing anti-ACE2 multispecific, e.g. bispecific antigen-binding proteins or anti-ACE2 bispecific antibodies. As such, an anti-ACE2 multispecific antigen-binding protein, e.g. an anti-ACE2 bispecific antigen-binding protein, targets or specifically binds said human ACE2 protein(s) and at least one other antigen of interest. In various aspects, the mouse model for testing anti-ACE2 bispecific antigen-binding proteins wherein the antigen-binding protein is capable of binding both human ACE2 and the antigen of interest comprises a nucleic acid sequence encoding a human ACE2 protein

In an embodiment, the testing of the monospecific or bispecific antigen-binding protein involves performing an assay or a study that allows determination of the effect of the antigen-binding protein on a cell expressing said human ACE2 protein. In another embodiment, the testing of the bispecific antigen-binding protein involves performing an assay or a study that allows determination of the effect of the antigen-binding protein on both the cell expressing said human ACE2 protein(s) and the cell expressing or comprising the antigen of interest, or the interaction between said ACE2-expressing cell and the cell expressing or comprising the antigen of interest. In one embodiment, the testing of the monospecific or bispecific antigen-binding protein involves performing an assay or a study that allows determination of the effect of the cell expressing said human ACE2 protein(s) on the cell expressing or comprising said antigen of interest. In one embodiment, such assay measures, e.g., the number of cells expressing the antigen of interest, immune response, cellular interactions, cellular cytotoxicity, cytokine release, cellular activation, cell proliferation, tumor growth or regression, changes in pathology, or the like. Various assays include but are not limited to measurements of complement-directed cytotoxicity (CDC), antibody-dependent cytotoxicity (ADCC), antibody-dependent cellular phagocytosis (ADCP), PBMC proliferation, CD69 activation, histological tissue analysis, analysis of tissue and cellular biomarkers (e.g., cells or tissue may be extracted from the mouse for the purpose of the assays, or analyzed by radiography, MRI, PET, SPECT, BLI, and fluorescence-based imaging modalities).

In some embodiments of the invention, in such a mouse model, the antigen of interest has been introduced into said mouse. The antigen of interest may be introduced by several methods known to those skilled in the art. Some nonlimiting methods include transgenesis, injection, infection, tissue or cell transplantation. The antigen of interest or a fragment thereof (e.g., a fragment that is recognized by the antigen-binding protein being tested) can be targeted to, or expressed by, particular cell types. In some embodiments, the antigen of interest is a human antigen of interest encoded by the mouse genome.

The antigen of interest may be a membrane-bound protein such that it is expressed only on cell surface. Alternatively, the antigen of interest or a fragment thereof (e.g., a fragment that is recognized by the antigen-binding protein being tested) may be displayed on the cell surface complexed with another protein or moiety. Some cell-surface antigens may associate with other proteins as co-receptor complexes, or bind or have affinity to extracellular molecules. Thus, the mouse model may be utilized to test bispecific antigen-binding molecules that interact with cells in various cell systems.

In various embodiment of the invention, the antigen-binding protein binds both human ACE2 and the antigen of interest in the mouse model. In one embodiment, the antigen of interest is a human antigen. In one embodiment, the antigen of interest is a primate antigen, e.g., a cynomolgus monkey antigen. In one embodiment, the antigen-binding protein is capable of binding the same antigen of interest of both human and monkey origin. In one embodiment, the antigen-binding protein is capable of binding both human and monkey ACE2.

In another embodiment of the invention, the mouse model is used to determine if a candidate bispecific antigen-binding protein is capable of blocking or affecting an antigen of interest which is an infectious disease associated antigen. In one embodiment of the invention, the mouse is infected with an infectious agent. In one embodiment of the invention, the infectious disease associated antigen is a viral antigen. In one aspect, the viral antigen is selected from the group consisting of a coronavirus, SARS-COV, and SARS-COV-2 antigen.

In some aspects of the invention, the ACE2-based bispecific antigen binding protein is a human ACE2 based antigen binding protein. In one embodiment, the antigen binding protein is an antibody, e.g., a human antibody, or an antigen-binding fragment thereof.

In some embodiments of the invention, the toxicity in the animal may be measured as an adverse event in the animal, e.g., change in body weight, appetite, digestive changes, changes in blood cell counts, splenomegaly, histological changes of the organs, change in liver enzyme function, changes in urinalysis, organ toxicity, hemorrhage, dehydration, loss of fur and scruffiness, or other signs of morbidity. One measure may be determination of antigen-binding protein cross-reactivity with irrelevant antigens, which, in one embodiment, can be detected by organ histology, specifically detection of antigen-binding protein in tissues or cell types that are not known to express the antigen of interest.

Use of Genetically Modified Non-Human Animals: The invention also provides various methods of using the genetically modified non-human animals described herein. In one embodiment, provided herein is a method of screening therapeutic drug candidates that target an antigen of interest comprising (a) providing or receiving a genetically modified mouse comprising multiple locations a nucleic acid sequence encoding an a human ACE2 protein, (b) introducing into said genetically modified mouse an antigen of interest, (c) contacting said mouse with a drug candidate of interest, wherein the drug candidate is directed against the human ACE2 and the antigen of interest, and (d) determining if the drug candidate is efficacious in preventing, reducing or eliminating cells characterized by the presence or expression of the antigen of interest. In various embodiments, the mouse expresses a functional human ACE2 protein(s) on the surface of its cells. In some embodiments, including when the drug candidate is a vaccine, the mouse may be contacted with the drug candidate before introducing the antigen of interest.

In various embodiments of the method described herein, introduction of the antigen of interest into the genetically modified mouse described herein may be accomplished by any methods known to those skilled in the art, which may include, without limitation, transgenesis, injection, infection, tissue or cell transplantation. As such, introduction may be achieved by expressing in the mouse the antigen of interest, which can comprise genetically modifying said mouse to express the antigen of interest. Alternatively, introduction may comprise introduction into said mouse a cell expressing the antigen of interest, e.g., as in cell or tissue transplantation. Introduction may also comprise infecting said mouse with the antigen of interest, e.g., as in bacterial or viral infection. In one embodiment, the antigen of interest may be a human antigen of interest. In another embodiment, it may be a bacterial or a viral antigen of interest.

The antigen of interest may be an infectious disease associated antigen, e.g., a bacterial or a viral antigen, as described in detail above.

In various embodiments of the methods described herein, the therapeutic candidate is capable of reducing, eliminating, or preventing a disease. In one embodiment, the disease is an infectious disease, and a therapeutic candidate is capable reducing, eliminating, or preventing a bacterial or a viral infection as compared to an agent that does not target the antigen of interest. In such an embodiment of the method, determination whether the drug candidate is efficacious in preventing, reducing or eliminating cells characterized by the presence or expression of the antigen of interest can be performed using a measure of bacterial or viral titers, induction of apoptotic markers in infected cells, etc.

Other methods of use of the human ACE2 mice of the present invention are also provided. For example, the non-human animal, e.g., a human ACE2 mouse, described herein may be used to study the mechanism of drug action. Prior to the development of the present animal, it was difficult to study the mechanism of drug action as such studies are not typically conducted in humans and primates, and often require an immunocompetent animal model. Understanding drug action mechanism can lead to development of better antibodies.

In yet other embodiments, the human ACE2 mouse can be used to study the effects of combination drug therapies in animal models, specifically combination drug therapies, e.g., where one drug is an antigen-binding protein that binds ACE2 and another drug is an agent that has previously been approved for a particular indication. Specific questions related to the dosing of the drugs and its effects can be addressed in an animal model prior to any human trials.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items, while only the terms “consisting of” and “consisting only of” are to be construed in the limitative sense.

Wherefore, I/we claim:

1. A genetically modified non-human animal comprising:
a genome containing an endogenous non-human ACE2 locus genetically modified to encode a complete human ACE2 gene.

2. The animal of claim 1 wherein the genome is genetically modified to encode a second complete human ACE2 gene.

3. The animal of claim 2 wherein the genome is genetically modified to encode a third complete human ACE2 gene and forth complete human ACE2 gene.

4. The animal of claim 1 wherein the animal is a rodent.

5. The animal of claim 4 wherein the rodent is a mouse.

6. The animal of claim 1 wherein the ACE2 gene encodes more than one ACE2 protein.

7. The animal of claim 6 wherein the more than one ACE2 protein includes at least one protein having an amino acid sequence that is at least 85 percent identical to one of SEQ ID No:2 to SEQ ID No:8.

8. The animal of claim 6 wherein the more than one ACE2 protein includes a first protein and a second protein, each having an amino acid sequence that is at least 85 percent identical to a respective sequence chosen from the group of SEQ ID No:2 to SEQ ID No:8.

9. The animal of claim 8 wherein the more than one ACE2 protein includes a third protein having an amino acid sequence that is at least 85 percent identical to a respective sequence chosen from the group of SEQ ID No:2 to SEQ ID No:8.

10. The animal of claim 9 wherein the more than one ACE2 protein includes a fourth protein having an amino acid sequence that is at least 85 percent identical to a respective sequence chosen from the group of SEQ ID No:2 to SEQ ID No:8.

11. The animal of claim 10 wherein the more than one ACE2 protein includes a sixth protein having an amino acid sequence that is at least 85 percent identical to a respective sequence chosen from the group of SEQ ID No:2 to SEQ ID No:8.

12. The animal of claim 1 wherein the genome is genetically modified to encode four complete human ACE2 genes, and an endogenous Tmprss2 gene is unmodified.

13. The animal of claim 1 wherein a LoxP gene flanks each of a 5′ and a 3′ end of a nucleic acid sequence of the human ACE2 gene.

14. The animal of claim 1, wherein the human ACE2 gene is expressed in a lung, kidney, spleen, stomach, liver, intestine, heart, and skeletal muscle of the animal.

15. The animal of claim 1, wherein the human ACE2 gene is expressed in a brain of the animal.

16. The animal of claim 15, wherein the human ACE2 gene is expressed in a cortex, striatum, middle brain, hippocampus, olfactory bulb, and cerebellum of the brain of the animal.

17. The animal of claim 1 wherein the genome is genetically modified to encode a second, a third, and a fourth complete human ACE2 gene;
the human ACE2 gene is at least 85 percent identical to SEQ ID No: 1;
the animal of is a mouse;
the human ACE2 gene encodes six protein variants;
an endogenous Tmprss2 gene is unmodified;
a LoxP gene flanks each of a 5′ and a 3′ end of a nucleic acid sequence of the human ACE2 gene; and
the human ACE2 gene is expressed in a lung, kidney, spleen, stomach, liver, intestine, heart, and skeletal muscle of the animal, and a cortex, striatum, middle brain, hippocampus, olfactory bulb, and cerebellum of a brain of the animal.

18. A transgenic rodent cell comprising:
a genome containing an endogenous non-human ACE2 locus genetically modified to encode a complete human ACE2 gene;
the genome is genetically modified to encode a second complete human ACE2 gene;
the human ACE2 gene is at least 85 percent identical to SEQ ID No: 1;
the human ACE2 gene encodes multiple protein variants;
an endogenous Tmprss2 gene is unmodified; and
a LoxP gene flanks each of a 5′ and a 3′ end of a nucleic acid sequence of the human ACE2 gene.

19. The cell from claim 18 wherein the cell is a zygote.

20. A method of creating a transgenic non-human animal comprising
microinjecting into fertilized mouse oocytes
a) a gene sequence that is at least 90 percent identical to SEQ ID No: 1,
b) Cas9-ribonuclear protein (RNP),
c) multiple gRNAs targeting mouse endogenous Ace2 locus, and
d) multiple single-stranded oligonucleotides.
